(19) United States
(12) Patent Application Publication
(10) Pub. No.: US 2009/0099176 A1
(43) Pub. Date: Apr. 16, 2009

(54) PYRROLOPYRIDINE-2-CARBOXYLIC ACID AMIDES

(76) Inventors: Thomas Martin Krulle, Oxford (GB); Robert John Rowley, Oxford (GB); Gerard Hugh Thomas, Oxford (GB)

Correspondence Address: OSI PHARMACEUTICALS, INC., 41 PINELAWN ROAD, MELVILLE, NY, 11747, US

(21) Appl. No.: 11/792,185

(22) PCT Filed: Dec. 2, 2005
(86) PCT No.: PCT/GB2005/050233

§ 371 Date: Nov. 10, 2008

Related U.S. Application Data
(60) Provisional application No. 60/632,461, filed on Dec. 2, 2004.

(51) Int. Cl. A61K 31/5377; C07D 471/04; C07D 413/02; A61K 31/497; A61P 9/10; C07D 401/14; A61K 31/437

(52) U.S. Cl. 514/234.5; 546/113; 544/127; 544/363; 514/300; 514/253.07

(57) ABSTRACT

Compounds represented by Formula (I) or pharmaceutically acceptable salts thereof, are useful in the prophylactic or therapeutic treatment of diabetes, hyperglycemia, hypercholesterolemia, hyperinsulinemia, hyperlipidemia, hypertension, atherosclerosis or tissue ischemia e.g. myocardial ischemia, and as cardioprotectants.

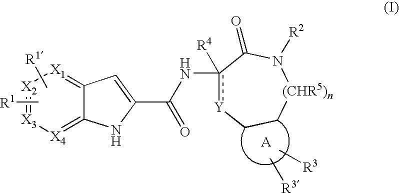

### BACKGROUND OF THE INVENTION

The present invention is directed to pyrrolopyridine-2-carboxylic acid amides. In particular, the present invention is directed to pyrrolopyridine-2-carboxylic acid amides that are inhibitors of glycogen phosphorylase.

Insulin dependent Type I diabetes and non-insulin dependent Type II diabetes continue to present treatment difficulties even though clinically accepted regimens that include diet, exercise, hypoglycemic agents, and insulin are available. Treatment is patient dependent, therefore there is a continuing need for novel hypoglycemic agents, particularly ones that may be better tolerated with fewer adverse effects.

The liver and certain other organs produce glucose by breaking down glycogen or by synthesizing glucose from small molecule precursors, thereby raising the blood sugar levels. The breakdown of glycogen is catalyzed by glycogen phosphorylase enzyme. Accordingly, inhibiting glycogen phosphorylase (“GP”) may lower the elevated blood sugar level in diabetic patients.

Similarly, hypertension and its associated pathologies such as, for example, atherosclerosis, lipidemia, hyperlipidemia and hypercholesterolemia have been associated with elevated insulin levels (hyperinsulinemia), which can lead to abnormal blood sugar levels. Furthermore, myocardial ischemia can result. Such maladies may be treated with hypoglycemic agents, including compounds that inhibit glycogen phosphorylase. The cardioprotective effects of glycogen phosphorylase inhibitors, for example following reperfusion injury, has also been described (see, for example, Ross et al., American Journal of Physiology. Heart and Circulatory Physiology, March 2004, 286(3), H1177-84). Accordingly, it is accepted that compounds that inhibit glycogen phosphorylase (see, for example, U.S. Pat. No. 6,297,269) are useful in the treatment of diabetes, hyperglycemia, hypercholesterolemia, hyperinsulinemia, hyperlipidemia, atherosclerosis or myocardial ischemia. Nevertheless, it would be desirable to obtain other novel compounds that inhibit glycogen phosphorylase.

R. Kurukulasuriya, J. T. Link, et al., Current Medicinal Chem., 10:99-121 (2003) describes “Prospects for Pharmacologic Inhibition of Hepatic Glucose Production.” R. Kurukulasuriya, J. T. Link, et al., Current Medicinal Chem., 10:123-153 (2003) describes “Potential Drug Targets and Progress Towards Pharmacologic Inhibition of Hepatic Glucose Production.”

U.S. Pat. No. 6,297,269 and European Patent No. EP 0832066 describe substituted N-(indole-2-carbonyl)amides and derivatives as glycogen phosphorylase inhibitors. U.S. Pat. Nos. 6,107,329 and 6,277,877 describe substituted N-(indole-2-carbonyl)glycinamides and derivatives as glycogen phosphorylase inhibitors. U.S. Pat. No. 6,399,601 describes bicyclic pyrrolyl amides as glycogen phosphorylase inhibitors. European Patent Application Nos. EP 0978276 and EP 1136071 describe inhibitors of human glycogen phosphorylase and their use. International Patent Publication No. WO 01/68055 describes glycogen phosphorylase inhibitors. U.S. Patent Application No. US2004/0002495 describes glycogen phosphorylase inhibitors. U.S. Pat. No. 5,952,322 describes a method of reducing non-cardiac ischemial tissue damage using glycogen phosphorylase inhibitors.

International Patent Publication No. WO 01/55146 describes arylamidines. International Patent Publication No. WO 01/62775 describes antiarrhythmic peptides. International Patent Publication No. WO 01/96346 describes tricyclic compounds. International Patent Publication No. WO 02/16314 describes substituted polyamine compounds. International Patent Publication No. WO 02/20475 describes serine protease activity inhibitors. International Patent Publication No. WO 02/40469 describes bombesin receptor antagonists. International Patent Publication No. WO 02/46159 describes guanidine and amidine derivatives. International Patent Publication No. WO 00/69815 describes ureido-substituted cyclic amine derivatives.

International Patent Publication No. WO 00/43384 describes aromatic heterocyclic compounds. International Patent Publication Nos. WO 02/26697 and WO 00/76970 describe aromatic derivatives. International Patent Publication No. WO 01/32622 describes indoles. European Patent Application No. EP 1101759 describes phenylazole compounds. European Patent Application No. EP 1179341 describes cyclic amino compounds. U.S. Pat. No. 6,037,325 describes substituted heterocyclic compounds. U.S. Pat. No. 5,672,582 describes 4-substituted cyclohexylamine derivatives. European Patent Application No. EP 1201239 describes cyclic amine CCR3 antagonists. International Patent Publication No. WO 98/25617 describes substituted aryl piperazines. U.S. Pat. No. 5,756,810 describes preparing 3-nitrobenzoate compounds.

U.S. Pat. No. 5,710,153 describes tetrazole compounds. U.S. Pat. Nos. 6,174,887 and 6,420,561 describe amide compounds. S. P. Hiremath et al., Acta Ciencia Indica, XVIII:397 (1992) describes the synthesis and biological activities of indolylthiosemicarbazides and semicarbazides. International Patent Publication No. WO 96/36595 describes 3,4-disubstituted phenylsulfonamides. U.S. Pat. No. 5,618,825 describes combinatorial sulfonamide libraries. European Patent Application No. EP 0810221 describes oxygen-containing heterocyclic derivatives. European Patent Application No. EP 0345990 describes polypeptide compounds. European Patent Application No. EP 0254545 describes diamine compounds.

International Patent Publication No. WO 97/31016 describes inhibitors of SH2-mediated processes. U.S. Pat. No. 6,034,067 describes serine protease inhibitors. International Patent Publication No. WO 97/17985 and U.S. Pat. No. 6,107,309 describe hemoregulatory compounds. U.S. Pat. No. 6,432,921 describes thrombin inhibitors. U.K. Patent Application No. GB 2292149 describes peptide inhibitors of pro-interleukin-1β converting enzyme. U.S. Pat. No. 5,821,241 describes fibrinogen receptor antagonists.

International Patent Publication No. WO 01/02424 describes peptide boronic acid compounds. U.S. Pat. Nos. 6,001,811, 5,869,455 and 5,618,792 describe oxadiazole, thiadiazole and triazole peptoids. U.S. Pat. Nos. 5,885,967, 6,090,787 and 6,124,277 describe thrombin inhibiting peptide derivatives. U.S. Pat. No. 6,455,529 describes adhesion receptor antagonists. U.S. Pat. No. 6,410,684 describes serine protease inhibitors.

International Patent Publication No. WO 01/94310 describes bis-heterocyclic alkaloids. U.S. Patent Publication No. 20030004162A1, European Patent Application No. EP 0846464, and International Publication No. WO 96/39384 describe glycogen phosphorylase inhibitors. International Patent Publication No. WO 97/28798 describes pyrrolidine derivatives. U.S. Pat. No. 5,346,907 describes amino acid analogs.

International Patent Application No. PCT/US2004/016243 (published after the priority date of the present invention) discloses pyrrolopyridine-2-carboxylic acid amide inhibitors of glycogen phosphorylase.

### SUMMARY OF THE INVENTION

Compounds represented by Formula (I):

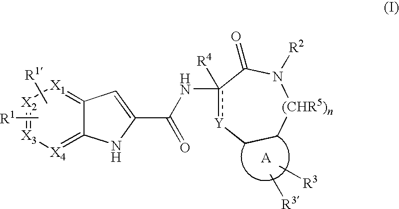

or stereoisomers or pharmaceutically acceptable salts thereof, are inhibitors of glycogen phosphorylase and are useful in the prophylactic or therapeutic treatment of diabetes, hyperglycemia, hypercholesterolemia, hyperinsulinemia, hyperlipidemia, hypertension, atherosclerosis or tissue ischemia e.g. myocardial ischemia, and as cardioprotectants.

### DETAILED DESCRIPTION OF THE INVENTION

The present invention provides a compound of Formula (I):

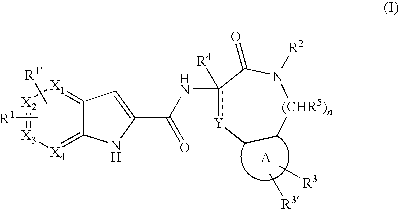

or a stereoisomer, or a pharmaceutically acceptable salt thereof, wherein:

one of X₁, X₂, X₃ and X₄ is N and the others are C;

[symbol] is a single or double bond;

when [symbol] is a single bond Y is CHR⁶, NH, O, S, SO₂, CHR⁶O, CHR⁶S, CHR⁶SO₂, CHR⁶CO or CH₂CHR⁶; and when [symbol] is a double bond Y is CR⁶ or N;

A is aryl or heteroaryl;

R¹ and R^{1′} are independently selected from hydrogen, halogen, hydroxy, cyano, C₁₋₆alkyl, C₁₋₆alkoxy, fluoromethyl, difluoromethyl, trifluoromethyl, C₂₋₆alkenyl, C₂₋₆alkynyl, aryl, —C₁₋₆alkylaryl, —C₁₋₆alkylheteroaryl and aryloxy;

R² is hydrogen, C₁₋₆alkyl optionally substituted by cyano, O—C₁₋₄alkyl-OR⁷, OR⁷, COOR⁷, CONR⁸R⁹, CONR⁸OR⁹, C(NH₂)═NOH, NR¹⁶R¹⁷, NHC(O)OR¹⁶, NHS(O)₂R¹⁸, NHC(O)R¹⁸, SR¹⁶, S(O)R¹⁸ or S(O)₂R¹⁸; or R² is C₁₋₄alkyl-CONR¹⁰R¹¹, C₂₋₆alkenyl, aryl, —C₁₋₆alkylaryl, —C₁₋₆alkylheterocyclyl or —C₁₋₆alkylheteroaryl;

R³ and R^{3′} are independently selected from hydrogen, halogen, hydroxy, cyano, C₁₋₄alkyl, C₁₋₄alkoxy, fluoromethyl, difluoromethyl, trifluoromethyl, ethenyl and ethynyl;

when [symbol] is a single bond R⁴ is hydrogen, C₁₋₆alkyl, aryl, or C₂₋₆alkenyl or when [symbol] is a double bond R⁴ is absent;

R⁵ and R⁶ are independently selected from hydrogen, C₁₋₆alkyl, aryl, C₂₋₆alkenyl, cyano, tetrazole, COOR¹², CONR¹²R¹³ and CONR¹²OR¹³;

R⁷, R⁸ and R⁹ are independently selected from hydrogen and C₁₋₄alkyl;

R¹⁰ and R¹¹ are independently selected from hydrogen, C₁₋₄alkyl optionally substituted by OR⁷, COOR⁷ or NR¹⁴R⁵, aryl, heteroaryl, C₃,cycloalkyl, heterocyclyl, —C₁₋₄alkylaryl, —C₁₋₄alkylheteroaryl, —C₁₋₄alkylC₃₋₇cycloalkyl or —C₁₋₄alkylheterocyclyl wherein any of the rings is optionally substituted by 1 or 2 substituents independently selected from halogen, hydroxy, cyano, C₁₋₄alkyl, C₁₋₄alkoxy, fluoromethyl, difluoromethyl and trifluoromethyl;

or R¹⁰ and R¹¹ together with the nitrogen to which they are attached form a 4- to 7-membered heterocycle optionally containing a further heteroatom selected from N and O, which heterocycle is optionally substituted by C₁₋₄alkyl, halo, OR⁷ or COR⁷, or two bonds on a ring carbon of the heterocycle optionally can form an oxo (═O) substituent;

R¹² and R¹³ are independently selected from hydrogen, C₁₋₄alkyl, aryl, —C₁₋₄alkylaryl and —C₁₋₄alkylheteroaryl;

or R¹² and R¹³ may be cyclised to form an optionally substituted 4- to 7-membered heterocycle;

R¹⁴ and R¹⁵ are independently selected from hydrogen and C₁₋₄alkyl;

or R¹⁴ and R¹⁵ together with the nitrogen to which they are attached form a 4- to 7-membered heterocycle optionally containing a further heteroatom selected from N and O, which heterocycle is optionally substituted by C₁₋₄alkyl, halo, OR⁷ or COR⁷, or two bonds on a ring carbon of the heterocycle optionally can form an oxo (═O) substituent;

R¹⁶ and R¹⁷ are independently selected from hydrogen and C₁₋₄alkyl;

R¹⁸ is C₁₋₄alkyl; and

n is 0 or 1.

The molecular weight of the compounds of Formula (I) is preferably less than 800, more preferably less than 600.

A is preferably a fused benzene, pyridine or thiophene ring, more preferably a fused benzene ring.

When [symbol] is a single bond R⁴ is preferably hydrogen.

In R² the C₁₋₆alkyl and C₁₋₄alkyl groups are preferably C₁₋₂alkyl.

When R² is C₁₋₆alkyl-CO—NR⁸R⁹ it is preferably CH₂—CO—NR⁸R⁹ and when R² is C₁₋₄alkyl-CO—NR¹⁰R¹¹ it is preferably CH₂—CO—NR¹⁰R¹¹.

When R¹⁰ and R¹¹, R¹² and R¹³ or R¹⁴ and R¹⁵ together with the nitrogen to which they are attached form a 4- to 7-membered heterocycle optionally containing a further heteroatom selected from N and O, said heterocycle is preferably optionally substituted by 1 or 2 substituents selected from C₁₋₄alkyl, halo, OR⁷ and COR⁷, or two bonds on a ring carbon of the heterocycle optionally can form an oxo (═O) substituent.

Suitably R⁵ represents hydrogen.

A specific group of compounds of Formula (I) which may be mentioned are those wherein R² is hydrogen, C₁₋₆alkyl optionally substituted by OR⁷, COOR⁷ or CONR⁸R⁹; or C₁₋₄alkyl-CONR¹⁰R¹¹, C₂₋₆alkenyl, aryl, —C₁₋₆alkylaryl or —C₁₋₆alkylheteroaryl.

A preferred group of compounds of the present invention are the compounds of Formula (Ia):

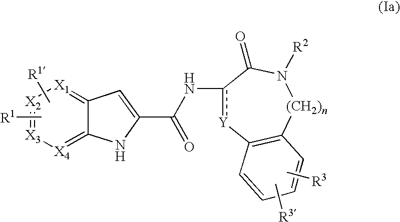

or a stereoisomer, or a pharmaceutically acceptable salt thereof, wherein:

one of X₁, X₂, X₃ and X₄ is N and the others are C;

[symbol] is a single or double bond;

when [symbol] is a single bond Y is CH₂, NH or O; and when [symbol] is a double bond Y is CH or N;

R¹ and R^{1′} are independently selected from hydrogen, halogen, hydroxy, cyano, C₁₋₄alkyl, C₁₋₄alkoxy, fluoromethyl, difluoromethyl, trifluoromethyl, ethenyl and ethynyl;

R² is hydrogen, C₁₋₄alkyl optionally substituted by cyano, O—C₁₋₄alkyl-OR⁴, OR⁴, COOR⁴, CONR⁵R⁶, CONR⁵OR⁶, C(NH₂)═N(OH), NR¹⁶R¹⁷, NHC(O)OR¹⁶, NHS(O)₂R¹⁸, NHC(O)R¹⁸, SR¹⁶, S(O)R¹⁸ or S(O)₂R¹⁸; or R² is C₁₋₄alkyl-CONR⁷R⁸ or —C₁₋₄alkylheterocyclyl;

R³ and R^{3′} are independently selected from hydrogen, halogen, hydroxy, cyano, C₁₋₄alkyl, C₁₋₄alkoxy, fluoromethyl, difluoromethyl, trifluoromethyl, ethenyl and ethynyl;

R⁴, R⁵ and R⁶ are independently selected from hydrogen and C₁₋₄alkyl;

R⁷ and R⁸ are independently selected from hydrogen, C₁₋₄alkyl optionally substituted by OR⁴, COOR⁴ or NR⁹R¹⁰, aryl, heteroaryl, C₃₋₇cycloalkyl, heterocyclyl, —C₁₋₄alkylaryl, —C₁₋₄alkylheteroaryl, —C₁₋₄alkylC₃₋₇cycloalkyl or —C₁₋₄alkylheterocyclyl wherein any of the rings is optionally substituted by 1 or 2 substituents independently selected from halogen, hydroxy, cyano, C₁₋₄alkyl, C₁₋₄alkoxy, fluoromethyl, difluoromethyl and trifluoromethyl;

or R⁷ and R⁸ together with the nitrogen to which they are attached form a 4- to 7-membered heterocycle optionally containing a further heteroatom selected from N and O, which heterocycle is optionally substituted by C₁₋₄alkyl, halo, OR⁴ or COR⁴, or two bonds on a ring carbon of the heterocycle optionally can form an oxo (═O) substituent;

R⁹ and R¹⁰ are independently selected from hydrogen and C₁₋₄alkyl;

or R⁹ and R¹⁰ together with the nitrogen to which they are attached form a 4- to 7-membered heterocycle optionally containing a further heteroatom selected from N and O, which heterocycle is optionally substituted by C₁₋₄alkyl, halo, OR⁴ or COR⁴, or two bonds on a ring carbon of the heterocycle optionally can form an oxo (═O) substituent;

R¹⁶ and R¹⁷ are independently selected from hydrogen and C₁₋₄alkyl;

R¹⁸ is C₁₋₄alkyl; and

n is 0 or 1.

When R² is C₁₋₄alkyl-CO—NR⁵R⁶ it is preferably CH₂—CO—NR⁵R⁶.

When R⁹ and R¹⁰ together with the nitrogen to which they are attached form a 4- to 7-membered heterocycle optionally containing a further heteroatom selected from N and O, said heterocycle is preferably optionally substituted by 1 or 2 substituents selected from C₁₋₄alkyl, halo, OR⁷ and COR⁷, or two bonds on a ring carbon of the heterocycle optionally can form an oxo (═O) substituent.

A specific group of compounds of Formula (Ia) which may be mentioned are those wherein R² represents hydrogen, C₁₋₄alkyl optionally substituted by OR⁴, COOR⁴ or CONR⁵R⁶; or C₁₋₄alkyl-CONR⁷R⁸.

In the compounds of Formulae (I) and (Ia):

Preferably one of X₂, X₃ and X₄ is N, more preferably X₃ is N.

[symbol] is preferably a single bond.

Y is preferably CH or CH₂, more preferably CH₂.

Preferably R¹ and R^{1′} are independently selected from hydrogen, halogen and cyano.

A preferred group of compounds are those where X₃ is N, one of R¹ and R^{1′} is hydrogen and the other is a 5-halo or 5-cyano group, especially a 5-chloro group.

Preferably R³ and R^{3′} are independently selected from hydrogen, halogen, C₁₋₄alkyl e.g. methyl, C₁₋₄alkoxy e.g. methoxy, and trifluoromethyl. Preferably at least one of R³ and R^{3′} is hydrogen.

n is preferably 0.

Specific compounds of the invention which may be mentioned are those included in the examples, as the free base or a pharmaceutically acceptable salt thereof.

While the preferred groups for each variable have generally been listed above separately for each variable, preferred compounds of this invention include those in which several or each variable in Formula (I) is selected from the preferred, more preferred, especially or particularly listed groups for each variable. Therefore, this invention is intended to include all combinations of preferred, more preferred, most preferred, especially and particularly listed groups.

As used herein, unless stated otherwise, “alkyl” as well as other groups having the prefix “alk” such as, for example, alkoxy, alkanyl, alkenyl, alkynyl, and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains having at least one unsaturated carbon-carbon bond.

As used herein, for example, “C₁₋₄alkyl” is used to mean an alkyl having 14 carbons—that is, 1, 2, 3, or 4 carbons in a straight or branched configuration. The term “C₁₋₆alkyl” may be interpreted in an analogous fashion.

The term “cycloalkyl” means carbocycles containing no heteroatoms, and include mono-, bi-, and tricyclic saturated carbocycles, as well as fused and bridged systems. Such fused ring systems can include one ring that is partially or fully unsaturated, such as a benzene ring, to form fused ring systems, such as benzofused carbocycles. Cycloalkyl includes such fused ring systems as spirofused ring systems. Examples of cycloalkyl and carbocyclic rings include C₃₋₇cycloalkyl such as cyclopropyl, cyclobutyl, cyclopentyl, cyclohexyl, and decahydronaphthalene, adamantane, indanyl, 1,2,3,4-tetrahydronaphthalene and the like.

The term “halogen” includes fluorine, chlorine, bromine, and iodine atoms.

The term “aryl” is well known to chemists. The preferred aryl groups are phenyl and naphthyl, more preferably phenyl.

The term “heteroaryl” is well known to chemists. The term includes 5- or 6-membered heteroaryl rings containing 1-4 heteroatoms chosen from oxygen, sulfur, and nitrogen in which oxygen and sulfur are not next to each other. Examples of such heteroaryl rings are furyl, thienyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridyl, pyridazinyl, pyrimidinyl, pyrazinyl, and triazinyl. The term “heteroaryl” includes heteroaryl rings with fused carbocyclic ring systems that are partially or fully unsaturated, such as a benzene ring, to form a benzofused hetaryl. For example, benzimidazole, benzoxazole, benzothiazole, benzofuran, quinoline, isoquinoline, quinoxaline, and the like.

Unless otherwise stated, the terms “heterocyclic ring” and “heterocycle” are equivalent, and include 4-8-membered saturated or partially saturated rings containing one or two heteroatoms chosen from oxygen, sulfur, and nitrogen. The sulfur and oxygen heteroatoms are not directly attached to one another. Any nitrogen heteroatoms in the ring may optionally be substituted with C₁₋₄alkyl. Examples of heterocyclic rings include azetidine, oxetane, tetrahydrofuran, tetrahydropyran, oxepane, oxocane, thietane, thiazolidine, oxazolidine, oxazetidine, pyrazolidine, isoxazolidine, isothiazolidine, tetrahydrothiophene, tetrahydrothiopyran, thiepane, thiocane, azetidine, pyrrolidine, piperidine, N-methylpiperidine, azepane, azocane, [1,3]dioxane, oxazolidine, piperazine, homopiperazine, morpholine, thiomorpholine, 1,2,3,6-tetrahydropyridine and the like. Other examples of heterocyclic rings include the oxidized forms of the sulfur-containing rings. Thus, tetrahydrothiophene-1-oxide, tetrahydrothiophene-1,1-dioxide, thiomorpholine-1-oxide, thiomorpholine-1,1-dioxide, tetrahydrothiopyran-1-oxide, tetrahydrothiopyran-1,1-dioxide, thiazolidine-1-oxide, and thiazolidine-1,1-dioxide are also considered to be heterocyclic rings. The term “heterocyclic” also includes fused ring systems and can include a carbocyclic ring that is partially or fully unsaturated, such as a benzene ring, to form benzofused heterocycles. For example, 3,4-dihydro-1,4-benzodioxine, tetrahydroquinoline, tetrahydroisoquinoline and the like.

Compounds described herein may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above Formula (I) is shown without a definitive stereochemistry at certain positions. The present invention includes all stereoisomers of Formula (I) and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of Formula (I) exists, the present invention includes any possible tautomers and pharmaceutically acceptable salts thereof, and mixtures thereof, except where specifically drawn or stated otherwise.

When the compound of Formula (I) and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The invention also encompasses a pharmaceutical composition that is comprised of a compound of Formula (I) in combination with a pharmaceutically acceptable carrier.

Preferably the composition is comprised of a pharmaceutically acceptable carrier and a non-toxic therapeutically effective amount of a compound of Formula (I) as described above (or a pharmaceutically acceptable salt thereof).

Moreover, within this preferred embodiment, the invention encompasses a pharmaceutical composition for the treatment of disease by inhibiting glycogen phosphorylase, resulting in the prophylactic or therapeutic treatment of diabetes, hyperglycemia, hypercholesterolemia, hyperinsulinemia, hyperlipidemia, hypertension, atherosclerosis or tissue ischemia e.g. myocardial ischemia comprising a pharmaceutically acceptable carrier and a non-toxic therapeutically effective amount of compound of Formula (I) as described above (or a pharmaceutically acceptable salt thereof).

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include arginine, betaine, caffeine, choline, N′N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

Since the compounds of Formula (I) are intended for pharmaceutical use they are preferably provided in substantially pure form, for example at least 60% pure, more suitably at least 75% pure especially at least 98% pure (% are on a weight for weight basis).

The pharmaceutical compositions of the present invention comprise a compound represented by Formula (I) (or a pharmaceutically acceptable salt thereof) as an active ingredient, a pharmaceutically acceptable carrier and optionally other therapeutic ingredients or adjuvants. The compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula (I), or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, sachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound represented by Formula (I), or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula (I). The compounds of Formula (I), or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each sachet or capsule preferably contains from about 0.05 mg to about 5 g of the active ingredient.

For example, a formulation intended for oral administration to humans may contain from about 0.5 mg to about 5 g of active agent, compounded with an appropriate and convenient amount of carrier material, which may vary from about 5 to about 95 percent of the total composition. Unit dosage forms will generally contain from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula (I), or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of Formula (I), or pharmaceutically acceptable salts thereof, may also be prepared in powder or liquid concentrate form.

Generally, dosage levels on the order of 0.01 mg/kg to about 150 mg/kg of body weight per day are useful in the treatment of the above-indicated conditions, or alternatively about 0.5 mg to about 7 g per patient per day. For example, diabetes and hyperglycemia may be effectively treated by the administration of from about 0.01 to 50 mg of the compound per kilogram of body weight per day, or alternatively about 0.5 mg to about 3.5 g per patient per day. Similarly, hypercholesterolemia, hyperinsulinemia, hyperlipidemia, hypertension, atherosclerosis or tissue ischemia e.g. myocardial ischemia may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

The compounds of Formula (I) may be used in the treatment of diseases or conditions in which glycogen phosphorylase plays a role.

Thus the invention also provides a method for the treatment of a disease or condition in which glycogen phosphorylase plays a role comprising a step of administering to a subject in need thereof an effective amount of a compound of Formula (I), or a pharmaceutically acceptable salt thereof.

Diseases or conditions in which glycogen phosphorylase plays a role include diabetes (including Type I and Type II, impaired glucose tolerance, insulin resistance and diabetic complications such as neuropathy, nephropathy, retinopathy and cataracts), hyperglycemia, hypercholesterolemia, hyperinsulinemia, hyperlipidemia, hypertension, atherosclerosis, tissue ischemia e.g. myocardial ischemia

The invention also provides a method for the treatment of hyperglycemia or diabetes comprising a step of administering to a subject in need thereof an effective amount of a compound of Formula (I), or a pharmaceutically acceptable salt thereof.

The invention also provides a method for the prevention of diabetes in a human demonstrating pre-diabetic hyperglycemia or impaired glucose tolerance comprising a step of administering to a subject in need thereof an effective prophylactic amount of a compound of Formula (I), or a pharmaceutically acceptable salt thereof.

The invention also provides a method for the treatment of hypercholesterolemia, hyperinsulinemia, hyperlipidemia, hypertension, atherosclerosis or tissue ischemia comprising a step of administering to a patient in need thereof an effective amount of a compound of Formula (I), or a pharmaceutically acceptable salt thereof.

The invention also provides a method of cardioprotection e.g. following reperfusion injury, comprising a step of administering to a subject in need thereof an effective amount of a compound of Formula (I), or a pharmaceutically acceptable salt thereof.

The invention also provides the use of a compound of Formula (I), or a pharmaceutically acceptable salt thereof, in the treatment of a condition as defined above.

The invention also provides the use of a compound of Formula (I), or a pharmaceutically acceptable salt thereof, in the manufacture of a medicament for the treatment of a condition as defined above.

In the methods of the invention the term “treatment” includes both therapeutic and prophylactic treatment.

The compounds of Formula (I), or pharmaceutically acceptable salts thereof, may be administered alone or in combination with one or more other therapeutically active compounds. The other therapeutically active compounds may be for the treatment of the same disease or condition as the compounds of Formula (I) or a different disease or condition. The therapeutically active compounds may be administered simultaneously, sequentially or separately.

The compounds of Formula (I) may be administered with other active compounds for the treatment of diabetes, for example insulin and insulin analogs, sulfonyl ureas and analogs, biguanides, α2 agonists, fatty acid oxidation inhibitors, α-glucosidase inhibitors, β-agonists, phosphodiesterase inhibitors, lipid lowering agents, antiobesity agents, amylin antagonists, lipoxygenase inhibitors, somostatin analogs, glucokinase activators, glucagon antagonists, insulin signalling agonists, PTP1B inhibitors, gluconeogenesis inhibitors, antilypolitic agents, GSK inhibitors, galanin receptor agonists, anorectic agents, CCK receptor agonists, leptin, CRF antagonists or CRF binding proteins.

The compounds of Formula (I) may also be administered in combination with thyromimetic compounds, aldose reductase inhibitors, glucocorticoid receptor antagonists, NHE-1 inhibitors or sorbitol dehydrogenase inhibitors.

The compounds of Formula (I) may exhibit advantageous properties compared to known glycogen phosphorylase inhibitors, for example, the compounds may exhibit improved solubility thus improving absorption properties and bioavailability.

All publications, including, but not limited to, patents and patent applications cited in this specification, are herein incorporated by reference as if each individual publication were specifically and individually indicated to be incorporated by reference herein as fully set forth.

In accordance with this invention, the compounds of Formula (I) can be prepared as outlined in Scheme 1 below wherein R¹, R^{1′}, R², R³, R^{3′}, R⁴, R⁵, X₁, X₂, X₃, X₄, Y, A, [symbol] and n are as defined above for Formula (I):

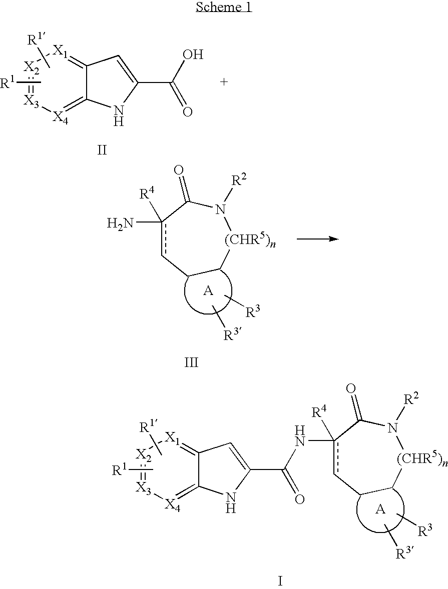

According to Scheme 1, the compounds of Formula (I) may be prepared by coupling the appropriate pyrrolopyridine-2-carboxylic acid of Formula (II), or a protected or activated derivative thereof, with the appropriate amine of Formula (III). Typically, the compound of Formula (II), or a protected or activated derivative thereof, is combined with compounds of Formula (III) in the presence of a suitable coupling agent. Examples of suitable coupling reagents are 1-(3-dimethylaminopropyl)-3-ethylcarbodiimide hydrochloride/hydroxybenzotriazole (EDCI/HOBt), 1,1-carbonyldiimidazole (CDI), dicyclohexylcarbodiimide/hydroxybenzotriazole (DCC/HOBt), O-(1H-benzotriazol-1-yl)-N,N,N′,N′-tetramethyluronium tetrafluoroborate (R. Knorr et al., Tetrahedron Lett., 1989, 30, 1927-1930) and polymer supported carbodiimide-1-hydroxybenzotriazole (for representative procedures, see for example, Argonaut Technical Note 501 available from Argonaut Technologies, Inc., Foster City, Calif.). The couplings are performed in an inert solvent, preferably an aprotic solvent at a temperature of about 0° C. to about 45° C. for about 1 to 72 h in the presence of a tertiary amine base such as diisopropylethylamine (DIPEA) or triethylamine. Exemplary solvents include acetonitrile, chloroform, dichloromethane, N,N-dimethylformamide (DMF) or mixtures thereof. Use of these coupling agents and appropriate selection of solvents and temperatures are known to those skilled in the art or can be readily determined from the literature. These and other exemplary conditions useful for coupling carboxylic acids are described in Houben-Weyl, Vol XV, part II, E. Wunsch, Ed., G. Thieme Verlag, 1974, Stuttgart, and M. Bodansky, Principles of Peptide Synthesis, Springer-Verlag, Berlin, 1984 and The Peptides, Analysis, Synthesis and Biology (Ed, E. Gross and J. Meienhofer), Vols 1-5, Academic Press NY 1979-1983.

Compounds of Formula (II) can be obtained by the synthesis outlined in Scheme 2 below.

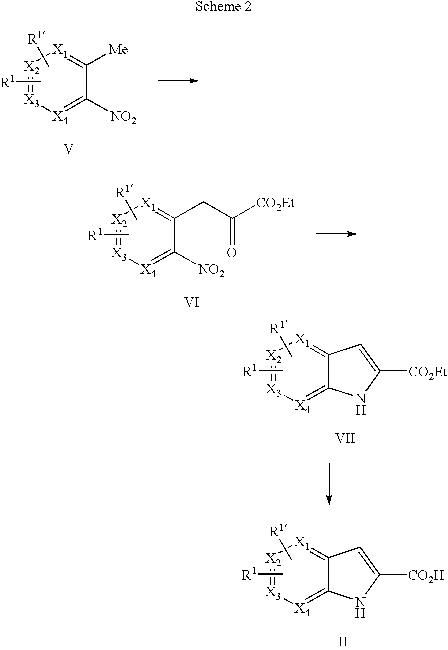

Compounds of Formula (VI) may be prepared by condensation of ortho methyl nitro compounds of Formula (V) with an oxalate ester in a solvent such as diethyl ether in the presence of a base such as potassium ethoxide or DBU. Compounds of Formula (VII) are prepared from compounds of Formula (VI) under reducing conditions, such as iron powder and ammonium chloride, or by hydrogenation in ethanol using palladium catalysis. Compounds of Formula (VII) undergo ester hydrolysis using aqueous alkali to give pyrrolopyridine-2-carboxylic acids of Formula (II). Further information on the conversion of compounds of Formula (V) to compounds of Formula (II) are described in the literature (Kermack, et al., J. Chem, Soc., (1921), 119, 1602; Cannon et al., J. Med. Chem., (1981), 24, 238; Julian et al., in Heterocyclic Compounds, Vol 3 (Wiley, New York, N.Y., 1962, R. C. Elderfield, Ed.) p 18.

Alternatively, the compound of Formula (VII) wherein X₂ is nitrogen can be prepared as outlined in Scheme 3.

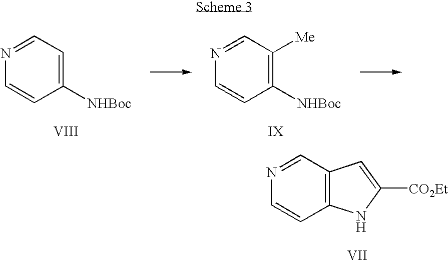

Deprotonation of compounds of Formula (VIII) with an organolithium such as n-butyllithium in a suitable solvent such as THF, followed by quenching with methyl iodide gives compounds of Formula (IX). Such compounds can undergo further deprotonation with tert-butyllithium, in a suitable solvent such as THF, followed by quenching with diethyl oxalate and subsequent heating of the intermediate under reflux in hydrochloric acid, to give compounds of Formula (VII).

Compounds of Formula (II) may also be prepared according to Scheme 4 by Heck coupling of an ortho-iodo aminopyridine (XI) followed by cyclisation at a temperature of between 100 to 150° C. in the presence of catalyst such as palladium acetate and a base such as DABCO in a solvent such as DMF (See Chen et al, J. Org. Chem. 1997, 62, 2676). The ortho-iodo aminopyridines (XI) can be made by direct iodination of the appropriate aminopyridine (X) using iodine in the presence of silver sulfate in a solvent such as ethanol at ambient temperature (see Sy, W., Synth. Commun., 1992, 22, 3215).

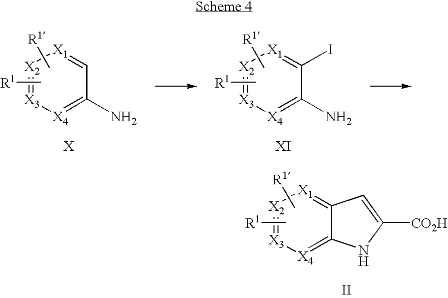

Alternatively compounds of Formula (XI) may be prepared according to Scheme 5 by deprotection of N-pivaloyl compounds (XII) by heating under reflux using hydrochloric acid. The N-pivaloyl compounds (XII) are in turn made by deprotonation of compounds of Formula (XIII) with an organolithium such as tert-butyllithium in a suitable solvent such as THF, followed by quenching with iodine at a low temperature. Compounds of formula (XIII) may be made by protection of commercially available aminopyridines (X) with trimethylacetyl chloride and a base such as triethylamine in a solvent such as dichloromethane.

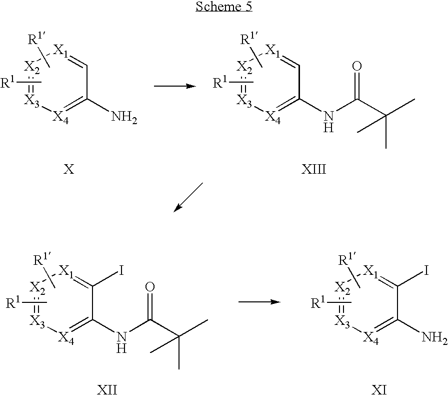

Alternatively compounds of Formula (XI) may be prepared according to Scheme 6 by deprotection of N—BOC protected compounds (XIV) using an acid such as trifluoroacetic acid in a solvent such as dichloromethane at ambient temperature. The N—BOC compounds (XIV) are in turn made by deprotonation of compounds of Formula (XV) with an organolithium such as n-butyllithium in the presence of N,N,N′,N′-tetramethylethylenediamine (TMEDA) in a suitable solvent such as ether at temperatures around −70° C. followed by the addition of iodine at temperatures around −10° C. The N—BOC aminopyridines (XV) are routinely made from the commercially available aminopyridines (X) using di-tert-butyldicarbonate by heating in a solvent such as 1,4-dioxane.

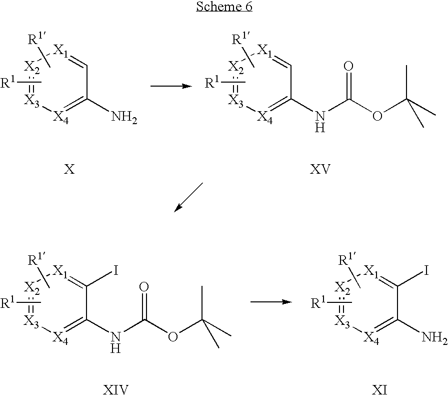

Protected or activated derivatives of the compounds of Formula (II) may be prepared by methods known to those skilled in the art.

Compounds of Formula (III) may be prepared by reacting an amine of Formula (XVI):

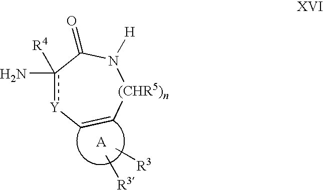

with R²-L, where L is a leaving group (for example chloro, bromo or iodo) in the presence of a base such as sodium hydride in a suitable solvent such as DMF.

Compounds of Formula (XVI) where A is phenylene, n is 0, R⁴ is hydrogen, Y is CH₂ and [symbol] is a single bond may be prepared from 3-amino-3,4-dihydroquinolin-2-(1H)-one (J. Med. Chem., (1985), 28, 1511-1516). Compounds of Formula (XVI) where A is phenylene and [symbol] is a double bond may be prepared by reductive cyclisation of a compound of Formula (XVII) using e.g. tin (II) chloride in HCl, followed by removal of the Boc protecting group, using e.g. trifluoroacetic acid. Compounds of Formula (XVII) may be prepared by reaction of a compound of Formula (XVIII) with a compound of Formula (XIX) in the presence of a base, e.g. tetramethylguanidine.

Compounds of Formula (XVIII) are commercially available or described in the literature.

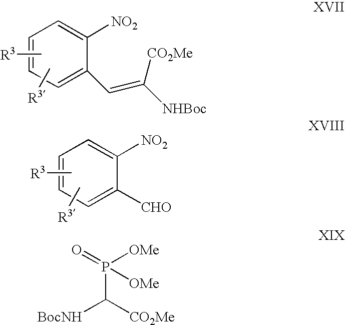

Compounds of Formula (III) wherein A is heteroarylene can be prepared from cyclisation of suitably functionalised heteroaryls when [symbol] is a single bond suitable methods for the synthesis of such compounds are as described in U.S. Patent Application No. US2004/0002495. For example when ring A is:

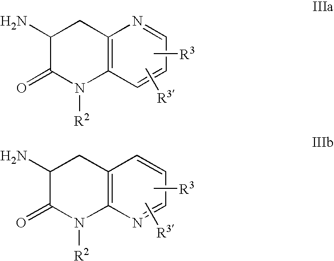

compounds of Formula (IIIa) and (IIIb) may be prepared from an appropriately substituted 3-nitro-2-methylpyridine or 2-aminopyridine according to Schemes 7 and 8.

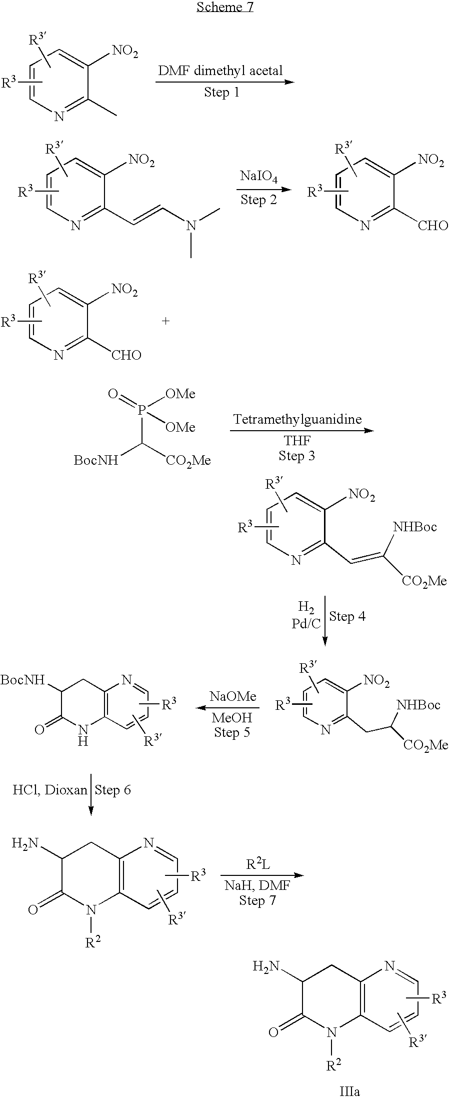

Steps 1 and 2 may be carried out according to the process described in Tetrahedron (1998), 54(23), 6311-6318. Step 3 may be carried out according to the method described in Synthesis (1992), 5, 487. Asymmetric hydrogenation reactions of olefins as shown in Step 4 are well known (see e.g. JACS, (1993), 115, 10125) and lead to homochiral final products. Step 5 may alternatively be carried out by hydrolysing the ester, activating the resulting acid with a carbodiimide such as EDCI or DCC, or by preparing an acid chloride, or activated ester such as an N-hydroxysuccinimide ester. Suitable bases are organic bases such as triethylamine or diisopropylamine (DIPEA) or 1,8-diazabicylo[5.4.0]undec-7-ene (DBU). In Step 6 alternative solvents such as dichloromethane or other acids such as trifluoroacetic acid may be used. In Step 7, L is a leaving group, for example Cl, Br, I, or OMs.

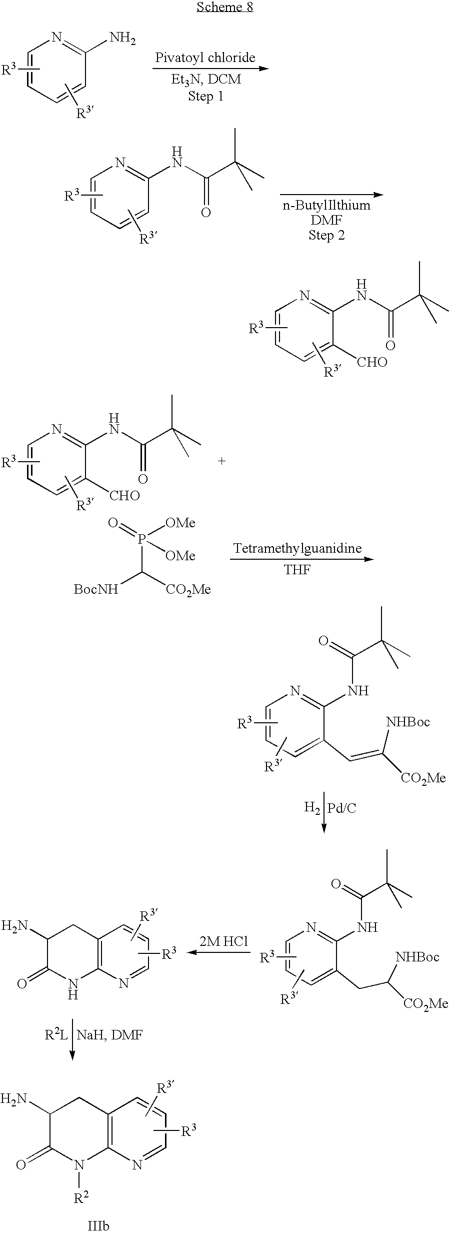

Steps 1 and 2 are described in JOC, (1983), 48, 3401-3408.

The processes described above and shown in Schemes 7 and 8 may also be used for the preparation of other isomeric pyridines or six membered heteroaryls containing more than one nitrogen.

Compounds of Formula (XVI) wherein A is heteroarylene and there is a bridgehead nitrogen, for example a compound of Formula (XVIa), may be prepared by cyclisation of a compound of Formula (XX):

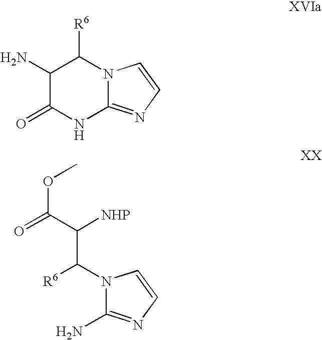

wherein P is an amino protecting group such as triphenylmethyl. The transformation may be induced by heating compounds of Formula (XX) under reflux in a solvent, for example, ethanol.

Compounds of Formula (XX) may be prepared from compounds of Formula (XX) by hydrogenation using a catalyst such as Pd/C at ambient temperature.

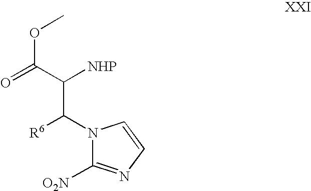

Compounds of Formula (XXII) may be prepared from compounds of Formulae (XXII) and (XXIII) using conditions known for the Mitsonobu reaction (Bull. Chem. Soc. Jpn., (1967), 40, 2380).

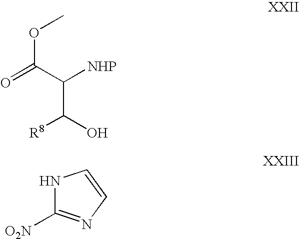

Compounds of Formulae (XXII) and (XXIII) are commercially available.

Compounds of Formula (XVI) wherein A is heteroarylene and there is a bridgehead heteroatom, for example, compounds of Formula (XVIb), may be made by analogous processes to that for making compounds of Formula (XVIa).

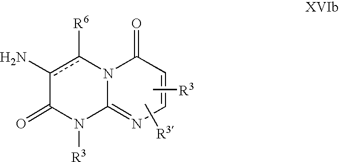

Various ring substituents in the compounds of the present invention, for example R³ and R^{3′}, may be introduced by standard aromatic substitution reactions or generated by conventional functional group modifications either prior to or following the processes described above. Such reactions and modifications include, for example, introduction of a substituent by means of an aromatic substitution reaction, reduction of substituents, alkylation of substituents and oxidation of substituents. Particular examples of aromatic substitution reactions include the introduction of a nitro group using concentrated nitric acid, the introduction of an acyl group using, for example, an acyl halide and Lewis acid (such as aluminium trichloride) under Friedal Crafts conditions; the introduction of an alkyl group using, for example, an alkyl halide and Lewis acid (such as aluminium trichloride) under Friedal Crafts conditions; and the introduction of a halogen group. Particular examples of modifications include the reduction of a nitro group to an amino group by for example, catalytic hydrogenation with a Nickel catalyst or treatment with iron in the presence of hydrochloric acid with heating; oxidation of alkylthio to alkylsulfinyl or alkylsulfonyl.

The compounds of Formula (I) may be prepared singly or as compound libraries comprising at least 2, for example 5 to 1,000 compounds and more preferably 10 to 100 compounds of Formula (I). Compound libraries may be prepared by a combinatorial “split and mix” approach or by multiple parallel synthesis using either solution or solid phase chemistry, using procedures known to those skilled in the art.

During the synthesis of the compounds of Formula (I), labile functional groups in the intermediate compounds, e.g. hydroxy, carboxy and amino groups, may be protected. The compounds of Formula (II) may be protected in the 1-position e.g. with an arylmethyl, acyl, alkoxycarbonyl, sulfonyl or silyl group. The protecting groups may be removed at any stage in the synthesis of the compounds of Formula (I) or may be present on the final compound of Formula (I). A comprehensive discussion of the ways in which various labile functional groups may be protected and methods for cleaving the resulting protected derivatives is given in for example, Protective Groups in Organic Chemistry, T. W. Greene and P. G. M. Wuts, (1991) Wiley-Interscience, New York, 2^{nd} edition.

Any novel intermediates as defined above, e.g. intermediates of Formula (III), are also included within the scope of the invention.

### EXPERIMENTAL

### Materials & Methods

Column chromatography was carried out on SiO₂ (40-63 mesh). LCMS data were obtained using a Waters Symmetry 3.5μ, C₁₈ column (2.1×30.0 mm, flow rate=0.8 mL/min) eluting with a (5% MeCN in H₂O)-MeCN solution containing 0.1% HCO₂H over 6 min and UV detection at 220 nm. Gradient information: 0.0-1.2 min: 100% (5% MeCN in H₂O); 1.2-3.8 min: ramp up to 10% (5% MeCN in H₂O)-90% MeCN; 3.8-4.4 min: hold at 10% (5% MeCN in H₂O)-90% MeCN; 4.4-5.5 min: ramp up to 100% MeCN; 5.5-6.0 min: return to 100% (5% MeCN in H₂O). The mass spectra were obtained employing an electrospray ionisation source in the positive (ES⁺) ion mode. NMR spectra were acquired at 27° C. on a Varian Mercury 400 spectrometer operating at 400 MHz or on a Bruker AMX2 500 spectrometer operating at 500 MHz. Mass directed purification was performed on a Micromass Platform LC with cone voltage 30 v, employing an electrospray ionisation source in the positive (ES⁺) ion mode, Waters 996 Photodiode Array Detector (210-390 nm), Xterra Prep MS, C₁₈, 5μ 19×50 mm columns, and a mobile Phase of MeCN+0.1% Formic Acid/H₂0+5% MeCN+0.1% Formic Acid

Abbreviations and acronyms: DABCO: 1,4-Diazabicyclo[2.2.2]octane; DCM: Dichloromethane; DIPEA: N,N-Diisopropylethylamine; DMA: N,N-Dimethylacetamide; DMF: N,N-Dimethylformamide; DMSO: Dimethylsulfoxide; DMSO: 4-(4,6-Dimethoxy[1.3.5]triazin-2-yl)-4-methylmorpholinium chloride hydrate; EDCI: 1-(3-Dimethylaminopropyl)-3-ethylcarbodiimide hydrochloride; EtOAc: Ethylacetate; GP: Glycogen Phosphorylase; HOBt: 1-Hydroxybenzotriazole; MgSO₄: Magnesium sulfate; NMP: N-Methylpyrrolidine; rt: Room temperature; RT: Retention time; TBAF: Tert-Butylammonium fluoride; THF: Tetrahydrofuran.

### Preparation 1: 3-(2-Chloro-5-nitropyridin-4-yl)-2-oxopropionic acid ethyl ester

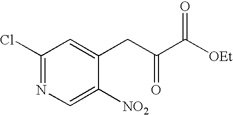

Route A: To a solution of potassium ethoxide (1.46 g, 17.4 mmol) in diethyl ether (80 mL) and ethanol (10 mL) under an argon atmosphere was added diethyl oxalate (2.4 mL, 17.4 mmol) and the mixture stirred at rt for 0.5 h. A solution of 2-chloro-4-methyl-5-nitropyridine (3.0 g, 17.4 mmol) in diethyl ether (20 mL) was added resulting in the formation of a dark green precipitate. The reaction was stirred at rt for 15 h, cooled to 0° C., filtered and washed with cold diethyl ether to give a dark green solid. The solid was dissolved in water (200 mL) and acidified to pH 4 with acetic acid to give an orange precipitate. The solid was collected by filtration and dried to give the title compound. m/z (ES⁺)=273 [M+H]⁺.

Route B: To a solution of 2-chloro-4-methyl-5-nitropyridine (1.0 g, 5.8 mmol) in diethyl oxalate (4.23 g, 29 mmol) under an argon atmosphere was added 1,8-diazabicyclo[5.4.0]undec-7-ene (0.95 mL, 6.4 mol). The mixture was stirred at rt for 1.5 h then diluted with t-butyl methyl ether (40 mL), water (30 mL) and acetic acid (11 mL). The organic layer was separated, washed with water, dried (MgSO₄) and evaporated to dryness. The resultant damp red solid residue was finally dried under high vacuum at 40-50° C. to give the title compound.

### Preparation 2: 5-Chloro-1H-pyrrolo[2,3-c]pyridine-2-carboxylic acid ethyl ester

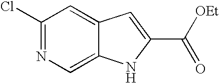

3-(2-Chloro-5-nitropyridin-4-yl)-2-oxopropionic acid ethyl ester (Preparation 1, 3.0 g, 11.0 mmol) was dissolved in ethanol (100 mL) and THF (50 mL). Iron powder (3.7 g, 66.0 mmol) and saturated ammonium chloride solution (50 mL) were added and the mixture heated under reflux for 2 h. The mixture was cooled, filtered through celite and washed several times with ethyl acetate. The organic layers were combined, washed with brine (100 mL), dried (MgSO₄) and concentrated in vacuo to give the title compound as a brown solid. δ_{H} (CD₃OD): 1.42 (3H, t), 4.44 (2H, q), 7.15 (1H, s), 7.70 (1H, s), 8.59 (1H, s); m/z (ES⁺)=225 [M+H]⁺.

### Preparation 3: 5-Chloro-1H-pyrrolo[2,3-]pyridine-2-carboxylic acid

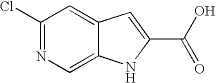

Route A: To a solution of 5-chloro-1H-pyrrolo[2,3-c]pyridine-2-carboxylic acid ethyl ester (Preparation 2, 1.78 g, 7.9 mmol) in ethanol (70 mL) was added sodium hydroxide solution (5.2 mL, 2M, 10.3 mmol) and the mixture heated under reflux for 2 h. The solvent was removed in vacuo and the solid dissolved in water (150 mL) and acidified to pH 4 with acetic acid to give the title compound as a brown solid that was isolated by filtration. δ_{H} (CD₃OD): 7.13 (1H, s), 7.68 (1H, s), 8.58 (1H, s); m/z (ES⁺)=197 [M+H]⁺.

Route B: A mixture of 6-chloro-4-iodopyridin-3-ylamine (Preparation 8, 0.33 g, 1.30 mmol), pyruvic acid (0.27 mL, 3.89 mmol), DABCO (0.44 g, 3.89 mmol) and palladium acetate (0.015 g, 0.07 mmol) in dry DMF was stirred vigorously and degassed with argon for 15 min. The reaction mixture was heated to 107° C. for 5 h. The reaction mixture was allowed to cool to rt and stirred for 16 h. The volatiles were removed under reduced pressure and the residue partitioned between EtOAc (100 mL) and water (50 mL). The layers were separated and the aqueous extracted with EtOAc (2×50 mL). The combined organics were extracted with aqueous NaOH (2M, 3×70 mL). The combined aqueous extracts were acidified to pH 4 by careful addition of glacial acetic acid, then extracted with EtOAc (3×60 mL). The combined organics were washed with brine (50 mL), dried (MgSO₄), filtered and concentrated in vacuo to give the title compound as a brown solid. RT=2.72 min, m/z (ES⁺)=197 [M+H]⁺

### Preparation 4: 5-Chloro-3-iodopyridin-2-ylamine

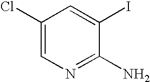

Silver sulfate (3.40 g, 10.9 mmol) and 2-amino-5-chloropyridine (1 g, 7.8 mmol) was added to a solution of iodine (2.76 g, 10.9 mmol) in ethanol (50 mL) and the reaction mixture stirred at rt for 72 h. The mixture was filtered, washed with methanol and the filtrate concentrated in vacuo. The residue was partitioned between saturated Na₂S₂O₃ solution (50 mL) and DCM (2×50 mL). The combined organics were dried (MgSO₄), concentrated in vacuo and purified by chromatography on silica gel eluting with DCM to give the title compound as a beige solid. δ_{H} (CDCl₃): 4.95 (2H, br s), 7.84 (1H, d), 7.98 (1H, d).

### Preparation 5: 5-Chloro-1H-pyrrolo[2,3-b]pyridine-2-carboxylic acid

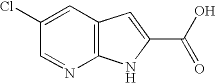

Pyruvic acid (0.43 ml, 6.24 mmol) was added to a solution of 5-chloro-3-iodopyridin-2-ylamine (Preparation 4, 500 mg, 2.08 mmol), palladium acetate (23 mg, 0.0 mmol) and DABCO (700 mg, 6.24 mmol) in anhydrous DMF (20 mL). The reaction mixture was degassed with argon for 20 min, then heated to 110° C. for 16 h. The solvent was removed in vacuo and the residue suspended in water (10 mL) and acetic acid (5 mL) and then filtered. The solid was dissolved in EtOAc (50 mL), extracted into 2N NaOH solution (50 mL) and the organic layer discarded. The aqueous solution was acidified with concentrated HCl and extracted into EtOAc (2×40 mL). The combined organics were dried (MgSO₄) and concentrated in vacuo to give the title compound as a beige solid. δ_{H} (CD₃OD): 7.14 (1H, s), 8.14 (1H, d), 8.35 (1H, d).

### Preparation 6: (6-Chloropyridin-3-yl)carbamic acid tert-butyl ester

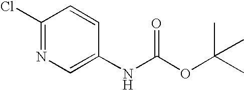

The title compound was prepared according to the method described in US 2002/0022624 A1. δ_{H} (CDCl₃): 1.52 (9H, s), 6.52 (1H, s), 7.26 (1H, d), 7.97 (1H, d), 8.23 (1H, d).

### Preparation 7: (6-Chloro-4-iodopyridin-3-yl)carbamic acid tert-butyl ester

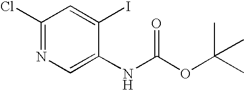

The title compound was prepared according to the method described in US 2002/0022624 A1 from the compound of Preparation 6. δ_{H} (CDCl₃): 1.54 (9H, s), 6.62 (1H, s), 7.72 (1H, s), 8.93 (1H, s).

### Preparation 8: 6-Chloro-4-iodopyridin-3-ylamine

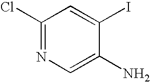

The title compound was prepared according to the method described in US 2002/0022624 A1 from the compound of Preparation 7. δ_{H} (CDCl₃): 4.12 (2H, br s), 7.60 (1H, s), 7.79 (1H, s).

### Preparation 9: N-(6-Chloropyridin-2-yl)-2,2-dimethyl propionamide

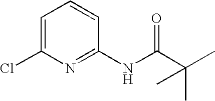

To a solution of 2-amino-6-chloropyridine (3.0 g, 23.3 mmol) in DCM (45 mL) under argon was added triethylamine (4.10 mL, 29.2 mmol) and the reaction cooled to 0° C. (ice bath). A solution of trimethylacetyl chloride (3.16 mL, 25.7 mmol) in DCM (10 mL) was added dropwise over 20 min before stirring for 30 min at 0° C. The reaction was brought up to rt and stirred for a further 5 h, then water (30 mL) was added. The organics were separated and washed with Na₂CO₃ solution (2×50 mL), dried (MgSO₄) and the solvent removed in vacuo. Purification by column chromatography (SiO₂, DCM) gave the title compound. m/z (ES⁺)=213.04 [M+H]⁺.

### Preparation 10: N-(6-chloro-3-iodopyridin-2-yl)-2,2-dimethyl propionamide

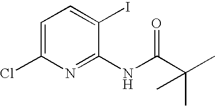

To a dry solution of N-(6-chloropyridin-2-yl)-2,2-dimethyl propionamide (Preparation 9, 8.0 g, 37.6 mmol) in THF (120 mL), cooled to −78° C., was added dropwise, a solution of tert-butyllithium in pentane (1.7M, 48.7 mL, 82.8 mmol) over 40 min. The reaction was stirred at −78° C. for 3 h before adding a solution of iodine (1.46 g, 45.1 mmol) in THF (40 mL) dropwise. The mixture was brought up to rt and stirred for 16 h. 2M HCl (30 mL) was added to the reaction, and after 20 min the solvent was removed in vacuo. Crude material was partitioned between EtOAc (200 mL) and water (150 mL). Organics were separated and washed with 10% sodium thiosulfate solution (4×100 mL) then NaHCO₃ solution (2×100 mL), dried (MgSO₄) and the solvent removed in vacuo. The residue was purified by column chromatography (SiO₂, CH₂Cl₂) to give the title compound. m/z (ES⁺)=338.93 [M+H]⁺.

### Preparation 11: 6-Chloro-3-iodopyridin-2-ylamine

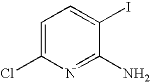

A suspension of N-(6-chloro-3-iodopyridin-2-yl)-2,2-dimethyl propionamide (Preparation 10, 5.0 g, 14.8 mmol) in 1M HCl was heated to reflux for 4.5 h. The reaction was cooled to rt and then extracted with diethyl ether (2×50 mL). The organics were washed with Na₂CO₃ solution (2×50 mL) before being dried (MgSO₄) and the solvent removed in vacuo. Purification by column chromatography (SiO₂, DCM) afforded the title compound. δ_{H} (CDCl₃): 7.76 (1H, d), 6.46 (1H, d), 5.43-5.20 (2H, br s).

### Preparation 12: 6-chloro-1H-pyrrolo[2,3b]pyridine-2-carboxylic acid

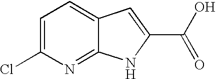

To a dry solution of 6-chloro-3-iodo-pyridin-2-ylamine (Preparation 11, 2.80 g, 11.0 mmol) in DMF (80 mL) under argon was added pyruvic acid (2.29 mL, 33.0 mmol), DABCO (3.70 g, 33.0 mmol) then palladium(II)acetate (124 mg, 0.55 mmol) and the mixture purged with argon for 20 min. The reaction was heated to 105° C. (bath temp.) for 3 h before being allowed to cool to rt. Solvent was removed in vacuo then crude material partitioned between EtOAc (100 mL) and water (75 mL). The organic layer was separated and washed with water (2×75 mL) before being extracted into 2M NaOH (2×75 mL). The aqueous layer was acidified to pH 3 with 2M HCl and extracted into EtOAc (2×100 mL). Organic layers were combined, dried (MgSO₄) and concentrated in vacuo. The residue was suspended in water and the filtrate removed to give the title compound. m/z (ES)=196.91 [M+H]⁺; RT=3.07 min.

### Preparation 13: 2-Chloro-5-iodopyridin-4-ylamine

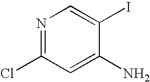

Silver sulfate (7.1 g, 22.8 mmol) and 4-amino-2-chloropyridine (4.06 g, 31.6 mmol) were added to a solution of iodine (5.65 g, 22.3 mmol) in ethanol (100 mL) and the reaction mixture stirred at rt for 72 h. The bright yellow suspension was filtered, washed with methanol and the filtrate concentrated in vacuo. The residue was partitioned between saturated Na₂CO₃ solution (200 mL) and EtOAc (200 ml). After separation the organic layer was washed with Na₂S₂O₃ solution (50 mL, 25%) and brine (50 mL), dried (MgSO₄), concentrated in vacuo and purified by chromatography on silica gel eluting with iso-hexane/EtOAc (3:1 to 2.5:1) to give the title compound. δ_{H} (CDCl₃): 4.81 (2H, br s), 6.63 (1H, s), 8.38 (1H, s); m/z (ES⁺)=254.86 [M+H]⁺; RT=2.51 min.

### Preparation 14: 6-Chloro-1H-pyrrolo[3,2-c]pyridine-2-carboxylic acid

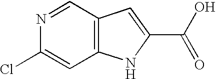

Pyruvic acid (0.86 ml, 12.4 mmol) was added to a solution of 2-chloro-5-iodopyridin-4-ylamine (Preparation 13, 1.05 mg, 4.13 mmol), palladium acetate (56 mg, 0.25 mmol) and DABCO (1.39 g, 12.4 mmol) in anhydrous DMF (30 mL). The reaction mixture was degassed with argon for 20 min, then heated to 145° C. for 2 h. The solvent was removed in vacuo and the residue taken up in water (200 mL). The suspension was made alkaline (pH 9-10) with dilute NaOH solution (1M) and filtered through Celite. After washing of the filtrate with EtOAc (50 mL) and ether (50 mL) the pH was adjusted to 3 with dilute HCl solution (IM). Extraction with EtOAc (5×50 mL), drying of the combined extracts (MgSO₄) and concentration gave the title compound. δ_{H} (d₆ DMSO): 7.24 (1H, s), 7.42 (1H, s), 8.80 (1H, s); m/z (ES⁻)=195.02 [M−H]⁻; RT=2.36 min.

### Preparation 15: 3-Amino-3,4-dihydro-1H-quinolin-2-one

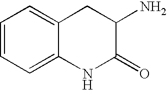

Diethyl acetamidomalonate (63.3 g, 0.29 mol) was added to a solution of sodium ethoxide (20.8 g, 0.31 mol) in ethanol (300 mL) and the reaction mixture heated to 50° C. for 15 min. 2-Nitrobenzyl chloride (50 g, 0.29 mol) and potassium iodide (2.4 g, 0.02 mol) were added and the reaction mixture heated at 60° C. for 3.5 h. Water (300 mL) was added to the reaction mixture and this was then concentrated by half in vacuo. The solid was filtered, washed with water and dried under vacuum affording 2-acetylamino-2-(2-nitrobenzyl)-malonic acid diethyl ester as a yellow solid. δ_{H} (d₆ DMSO): 1.13 (6H, t), 1.84 (3H, s), 3.81 (2H, s), 4.04-4.13 (4H, m), 7.22 (1H, dd), 7.50 (1H, td), 7.62 (1H, td), 7.86 (1H, dd), 8.11 (1H, s).

Saturated ammonium chloride solution (50 mL) and iron powder (19 g, 341 mmol) were added to a solution of 2-acetylamino-2-(2-nitrobenzyl)malonic acid diethyl ester (30 g, 85.1 mmol) in ethanol (200 mL) and THF (100 mL). The reaction mixture was heated to reflux for 3.5 h, then filtered through celite and washed several times with methanol. The solvent was removed in vacuo and the residue partitioned between water (300 mL) and EtOAc (3×150 mL). The combined organic fractions were dried (MgSO₄) and concentrated in vacuo affording 3-acetylamino-2-oxo-1,2,3,4-tetrahydro-quinoline-3-carboxylic acid ethyl ester as a beige solid. m/z (ES⁺) 277 [M+H]⁺; RT=2.70 min.

3-Acetylamino-2-oxo-1,2,3,4-tetrahydroquinoline-3-carboxylic acid ethyl ester (18.5 g, 67.0 mmol) was dissolved in concentrated hydrochloric acid (100 mL) and heated to reflux for 3 h. The reaction mixture was diluted with water (300 mL) and extracted into EtOAc (2×150 mL). The combined organic fractions were discarded and the aqueous phase was basified with sodium hydroxide solution (12M) and extracted into EtOAc (3×150 mL). The combined organic fractions were washed with brine (100 mL), dried (MgSO₄) and concentrated in vacuo affording the title compound as a brown solid. m/z (ES⁺) 163 [M+H]⁺; RT=2.44 min.

### Preparation 16: 3-Amino-1-[2-tert-butyldimethylsilanyloxy)ethyl]-3,4-dihydro-1H-quinolin-2-one

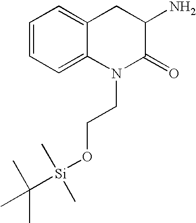

Sodium hydride (151 mg, 3.78 mmol) was added to a suspension of 3-amino-3,4-dihydro-1H-quinolin-2-one hydrochloride (Preparation 15, 300 mg, 1.51 mmol) in DMF (10 mL) at 0° C. over a period of 5 min. After 1 h, (2-bromoethoxy)tert-butyldimethylsilane (0.39 mL, 1.81 mmol) was added and the reaction mixture stirred at rt for 16 h, then 60° C. for 3 h. The reaction mixture was quenched with hydrochloric acid solution (1M, 3 mL) and the solvent removed in vacuo. The residue was partitioned between saturated NaHCO₃ solution (20 mL) and DCM (3×30 mL). The combined organic fractions were dried (MgSO₄), concentrated in vacuo and purified by chromatography on silica gel eluting with methanol:DCM (1:19) affording the title compound as a yellow oil. δ_{H} (CDCl₃): 0.00 (3H, s), 0.02 (3H, s), 0.86 (9H, s), 1.85 (2H, br s), 2.84 (1H, t), 3.05 (1H, dd), 3.56 (1H, dd), 3.83-3.98 (3H, m), 4.15-4.21 (1H, m), 7.02 (1H, td), 7.18 (1H, d), 7.22-7.30 (2H, m).

### Preparation 17: 3-Amino-1-tetrahydrofuran-2-ylmethyl)-3,4-dihydro-1H-quinolin-2-one

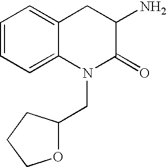

Sodium hydride (169 mg, 4.23 mmol) was added to a suspension of 3-amino-3,4-dihydro-1H-quinolin-2-one hydrochloride (Preparation 15, 400 mg, 2.01 mmol) in DMF (10 mL) at 0° C. over a period of 5 min. After 1 h, tetrahydrofurfuryl bromide (0.28 mL, 2.21 mmol) was added, and the reaction mixture stirred at rt for 16 h. The solvent was removed in vacuo and the residue partitioned between saturated K₂CO₃ solution (30 mL) and EtOAc (3×40 mL). The combined organic fractions were dried (MgSO₄), concentrated in vacuo and purified by chromatography on silica gel eluting with methanol:DCM (1:24) affording the title compound as a yellow oil. m/z (ES⁺) 247 [M+H]⁺.

### Preparation 18: 3-Amino-1-[2-(2-methoxyethoxy)ethyl]-3,4-dihydro-1H-quinolin-2-one

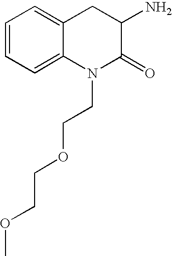

Sodium hydride (169 mg, 4.23 mmol) was added to a suspension of 3-amino-3,4-dihydro-1H-quinolin-2-one hydrochloride (Preparation 15, 400 mg, 2.01 mmol) in DMF (10 mL) at 0° C. over a period of 5 min. After 1 h 1-bromo-2-(2-methoxyethoxy)ethane (0.30 mL, 2.21 mmol) was added, and the reaction mixture stirred at rt for 5 days. The solvent was removed in vacuo and the residue partitioned between saturated K₂CO₃ solution (40 mL) and EtOAc (3×40 mL). The combined organic fractions were dried (MgSO₄), concentrated in vacuo and purified by chromatography on silica gel eluting with methanol:DCM (1:19) affording the title compound as a yellow oil. δ_{H} (CDCl₃): 1.99 (2H, br s), 2.85 (1H, t), 3.06 (1H, dd), 3.37 (3H, s), 3.51-3.68 (5H, m), 3.74 (2H, t), 4.07 (1H, dt), 4.25 (1H, dt), 7.03 (1H, td), 7.18-7.30 (3H, m).

### Preparation 19: 3-Amino-7-chloro-3,4-dihydro-1H-quinolin-2-one

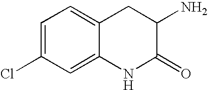

Route A: By a similar procedure to Preparation 15, the title compound was prepared using 4-chloro-2-nitrobenzyl chloride affording a peach solid. δ_{H} (CD₃OD): 2.85 (1H, t), 3.11 (1H, dd), 3.61 (1H, dd), 6.92 (1H, d), 6.99 (1H, dd), 7.20 (1H, d).

Route B: To a solution of 2-tert-butoxycarbonylamino-3-[4-chloro-2-(2,2-dimethylpropionylamino)phenyl]propionic acid methyl ester (Preparation 49, 1.23 g, 2.98 mmol) in THF (50 mL) was added dilute hydrochloric acid (2N, 50 mL) and the mixture stirred under reflux for 72 h. After cooling to rt and concentration in vacuo the residue was distributed between saturated sodium carbonate solution (200 mL) and EtOAc (200 mL). The layers were separated and the aqueous layer extracted with EtOAc (2×150 mL). Washing of the combined EtOAc fractions with saturated sodium carbonate solution (100 mL) and brine (100 mL) gave a solution which was concentrated after drying (MgSO₄). Purification of the residue by flash chromatography on silica gel (eluent: DCM/methanol: 9/1, 0.5% triethylamine) gave the title compound as a colourless solid. δ_{H} (d₄ MeOH): 2.85 (1H, t), 3.11 (1H, dd), 3.61 (1H, dd), 6.92 (1H, d), 6.99 (1H, dd), 7.20 (1H, d).

### Preparation 20: 3-Amino-7-trifluoromethyl-3,4-dihydro-1H-quinolin-2-one hydrochloride

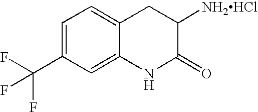

By a similar procedure to Preparation 15, the title compound was prepared using 2-nitro-4-(trifluoromethyl)benzyl chloride affording a white solid. δ_{H} (d₆ DMSO): 3.19 (1H, t), 3.34 (1H, dd), 4.26-4.31 (1H, m), 7.25 (1H, s), 7.34 (1H, dd), 7.52 (1H, d), 8.72 (3H, s), 11.00 (1H, s).

### Preparation 21: 3-Amino-6,7-dimethoxy-3,4-dihydro-1H-quinolin-2-one

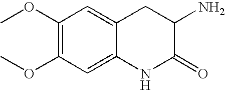

By a similar procedure to Preparation 15, the title compound was prepared using 4,5-dimethoxy-2-nitrobenzyl bromide affording a yellow solid. δ_{H} (CD₃OD): 2.75 (1H, t), 2.96 (1H, dd), 3.51 (1H, dd), 3.75 (6H, s), 6.49 (1H, s), 6.78 (1H, s).

### Preparation 22: 3-Amino-5-fluoro-3,4-dihydro-1H-quinolin-2-one

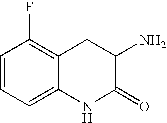

By a similar procedure to Preparation 15, the title compound was prepared using 2-fluoro-6-nitrobenzyl bromide affording a pink solid. m/z (ES⁺) 181 [M+H]⁺.

### Preparation 23: 3-Amino-6-methyl-3,4-dihydro-1H-quinolin-2-one

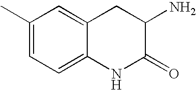

By a similar procedure to Preparation 15, the title compound was prepared using 5-methyl-2-nitrobenzyl chloride affording a tan solid. m/z (ES⁺) 177 [M+H]⁺.

### Preparation 24: 3-(R)-Amino-3,4-dihydro-1H-quinolin-2-one hydrochloride

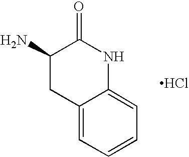

The title compound was prepared according to the method of Davis et. al. (J. Med. Chem., 1972, 15, 325). δ_{H} (d₆ DMSO): 7.30-7.19 (2H, m), 7.04-6.93 (2H, m), 4.26-4.14 (1H, m), 3.29-3.07 (2H, m).

### Preparation 25: 3-(S)-Amino-3,4-dihydro-1H-quinolin-2-one hydrochloride

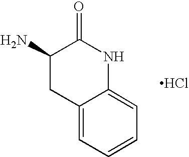

The title compound was prepared according to Preparation 24. δ_{H} (d₆ DMSO): 7.30-7.20 (2H, m), 7.03-6.91 (2H, m), 4.26-4.14 (1H, m), 3.28-3.09 (2H, m).

### Preparation 26: (3-(R)-Amino-2-oxo-3,4-dihydro-2H-quinolin-1-yl)-acetic acid methyl ester

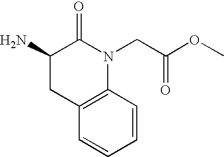

To a solution of 3-(R)-amino-3,4-dihydro-1H-quinolin-2-one hydrochloride (Preparation 24, 50 mg, 0.25 mmol) in DMF (5 mL) under argon, cooled to 0° C. (ice bath) was added sodium hydride (60% in mineral oil, 25 mg, 0.63 mmol), portion-wise, and the reaction stirred for 30 min. Methyl bromoacetate (26 μL, 0.28 mmol) was added and the mixture stirred at 0° C. for 1 h before warming to rt for 1.5 h. Conc.HCl (1 mL) was added to the reaction, which was stirred for 10 min before concentrating the solvent in vacuo. The residue was dissolved in DCM (30 mL) and washed with NaHCO₃ solution (2×10 mL) before being dried (MgSO₄) and the solvent removed in vacuo. Purification by column chromatography (SiO₂, 95:5 CH₂Cl₂/MeOH) afforded the title compound. m/z (ES⁺)=235.04 [M+H]⁺; RT=2.05 min.

### Preparation 27: (3-(S)-Amino-2-oxo-3,4-dihydro-2H-quinolin-1-yl)-acetic acid methyl ester

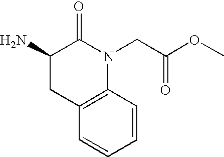

The title compound was prepared from 3-(S)-amino-3,4-dihydro-1H-quinolin-2-one hydrochloride (Preparation 25) according to Preparation 26. m/z (ES⁺)=235.03 [M+H]⁺; RT=1.97 min.

### Preparation 28: 3-(R)-Amino-1-[2-tert-butyl-dimethyl-silanyloxy)-ethyl]-3,4-dihydro-1H-quinolin-2-one

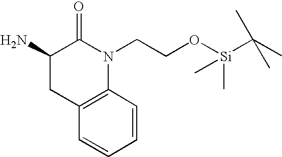

To a suspension of 3-(R)-amino-3,4-dihydro-1H-quinolin-2-one hydrochloride (Preparation 24, 199 mg, 1.0 mmol) in DMF (10 mL), cooled to 0° C. (ice bath), was added sodium hydride (60% suspension in mineral oil, 100 mg, 2.5 mmol), portion wise over 5 min. The reaction was stirred at 0° C. for 1 h before adding (2-bromoethoxy) tert-butyldimethylsilane (260 μL, 1.2 mmol). The mixture was brought up to rt then heated to 60° C. (bath temp) for 3 h. Solvent was concentrated in vacuo then crude residue taken into EtOAc (50 mL). Organics were washed with NaHCO₃ solution (2×30 mL) then brine (30 mL) before being dried (MgSO₄) and solvent removed in vacuo. Purification by column chromatography (SiO₂, 9:1 DCM/MeOH) afforded the title compound. δ_{H} (CDCl₃): 7.31-7.15 (3H, m), 7.07-7.0 (1H, m), 4.23-4.14 (1H, m), 4.0-3.83 (3H, m), 3.6-3.50 (1H, m), 3.06 (1H, dd), 2.90-2.80 (1H, m), 1.86 (9H, s), 0.04-0.00 (6H, m); m/z (ES⁺)=321.13 [M+H]⁺; RT=3.04 min.

### Preparation 29: 3-(R)-Amino-1-[2-tert-butyl-dimethyl-silanyloxy)-ethyl]-3,4-dihydro-1H-quinolin-2one

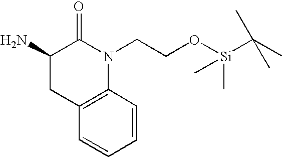

The title compound was prepared from 3-(S)-amino-3,4-dihydro-1H-quinolin-2-one hydrochloride (Preparation 25) according to Preparation 28. δ_{H} (CDCl₃): 7.31-7.15 (3H, m), 7.07-7.0 (1H, m), 4.23-4.14 (1H, m), 4.0-3.83 (3H, m), 3.6-3.50 (1H, m), 3.06 (1H, dd), 2.90-2.80 (1H, m), 1.86 (9H, s), 0.04-0.00 (6H, m); m/z (ES⁺)=321.15 [M+H]⁺; RT=3.09 min.

### Preparation 30: (R)-5-Chloro-1H-pyrrolo[2,3-c]pyridine-2-carboxylic acid{1-[2-(tert-butyldimethylsilanyloxy)ethyl]-2-oxo-1,2,3,4-tetrahydroquinolin-3-yl}amide

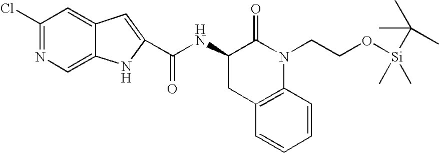

To a solution of 3-(R)-Amino-1-[2-(tert-butyl-dimethyl-silanyloxy)-ethyl]-3,4-dihydro-1H-quinolin-2-one (Preparation 28, 130 mg, 0.41 mmol) in DMF (5 mL) was added 5-chloro-1H-pyrrolo[2,3-c]pyridine-2-carboxylic acid (Preparation 3, 80 mg, 0.41 mmol) followed by DIPEA (177 μL, 1.01 mmol) and HOBt (68 mg, 0.45 mmol). After 5 min EDCI (93 mg, 0.49 mmol) was added and the reaction stirred for 16 h at rt. Solvent was concentrated in vacuo and the residue partitioned between EtOAc (50 mL) and water (30 mL). Organics were washed with NaHCO₃ solution (2×30 mL) and brine (30 mL) then dried (MgSO₄), and solvent removed in vacuo. Purification by column chromatography (SiO₂, 95:5 DCM/MeOH) afforded the title compound. δ_{H} (CDCl₃): 10.2 (1H, s), 8.69 (1H, s), 7.80 (1H, m), 7.60 (1H, s), 7.4-7.24 (2H, m), 7.16-7.09 (1H, m), 6.97 (1H, s), 4.76-4.66 (1H, m), 4.30-4.23 (1H, m), 4.09-3.86 (3H, m), 3.67-3.59 (1H, m), 2.99-2.90 (1H, m), 1.87 (9H, s), 0.04 (3H, s), 0.00 (3H, s); m/z (ES⁺)=499.09 [M+H]⁺; RT=4.23 min.

### Preparation 31: (S)-5-Chloro-1H-pyrrolo[2,3-c]pyridine-2-carboxylic acid{1-[2-(tert-butyldimethylsilyloxy)ethyl]-2-oxo-1,2,3,4-tetrahydroquinolin-3-yl}amide

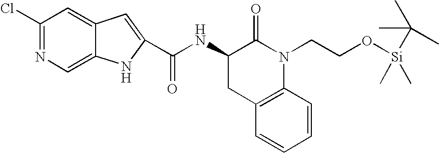

The title compound was prepared from 3-(R)-Amino-1-[2-(tert-butyl-dimethyl-silanyloxy)-ethyl]-3,4-dihydro-1H-quinolin-2-one (Preparation 29) according to Preparation 30. δ_{H} (CDCl₃): 9.76 (1H, s), 8.70 (1H, s), 7.69 (1H, m), 7.63 (1H, s), 7.40-7.23 (2H, m), 7.14-7.09 (1H, m), 6.69 (1H, s), 4.71-4.63 (1H, m), 4.30-4.23 (1H, m), 4.07-3.86 (3H, m), 3.68-3.60 (1H, m), 2.96-2.84 (1H, m), 1.86 (9H, s), 0.04 (3H, s), 0.00 (3H, s).

### Preparation 32: 4-Fluoro-2-nitrobenzaldehyde

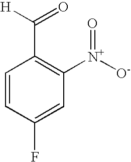

To a solution of 2-nitro-4-fluorotoluene (5.0 g, 32.2 mmol) in DMF (5 mL) was added DMF.DMA (12.84 mL, 97 mmol) and the reaction heated to 135° C. (bath temp) for 20 h. The mixture was cooled to rt before being added to a stirring solution of sodium periodate (6.9 g, 97 mmol) in water/DMF (23 mL: 12 mL) via cannula. The reaction was stirred at rt for 3 h then filtered and the precipitate washed with toluene (200 mL). The filtrate was separated and the organics washed with water (3×100 mL) and dried (MgSO₄) before removing the solvent in vacuo. Purification by column chromatography (SiO₂, 9:1 hexane/EtOAc) afforded the title compound. δ_{H} (CDCl₃): 10.36 (1H, s), 8.03 (1H, dd), 7.81 (1H, dd), 7.49 (1H, m).

### Preparation 33: 2-tert-Butoxycarbonylamino-3-(4-fluoro-2-nitrophenyl)acrylic acid methyl ester

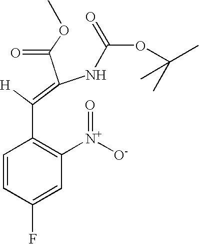

To a solution of (+/−)-BOC-α-phosphonoglycine trimethyl ester (1.66 g, 5.59 mmol) in THF (20 mL), cooled to −78° C. was added a solution of tetramethylguanidine (670 μL, 5.34 mmol) in THF (6 mL), dropwise. The reaction was stirred for 20 min before addition of a solution of 4-fluoro-2-nitro benzaldehyde (Preparation 32, 860 mg, 5.09 mmol) in THF (12 mL) via cannula. The reaction was stirred at rt for 16 h before concentrating the solvent in vacuo. The residue was taken into EtOAc (100 mL) and washed with water (2×30 mL) then brine (30 mL) before being dried (MgSO₄) and the solvent removed in vacuo. Purification by column chromatography afforded the title compound. δ_{H} (CDCl₃): 7.83 (1H, dd), 7.55 (1H, dd), 7.49 (1H, s), 7.33-7.25 (1H, m), 3.88 (3H, s), 1.31 (9H, s); m/z (ES⁺)=241.06 [M+H]⁺; RT=3.58 min.

### Preparation 34: (7-Fluoro-2-oxo-1,2,3,4-tetrahydroquinolin-3-yl) carbamic acid tert-butyl ester

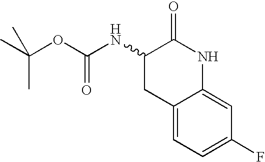

To a solution of 2-tert-butoxycarbonylamino-3-(4-fluoro-2-nitrophenyl)acrylic acid methyl ester (Preparation 33, 1.65 g, 4.85 mmol) in ethanol (80 mL) was added Palladium (10%) on carbon (516 mg, 0.48 mmol) and the reaction stirred under an atmosphere of hydrogen for 16 h. The mixture was filtered through celite, then 25% sodium methoxide in methanol (1.1 mL, 4.85 mmol) was added and the reaction stirred for a further 16 h. Water (50 mL) was added and the organics extracted into EtOAc (2×200 mL), washed with brine (2×50 mL) and dried (MgSO₄). Solvent was removed in vacuo, then trituration from diethyl ether/hexane afforded the title compound. δ_{H} (CD₃OD): 7.23-7.14 (1H, m), 6.74-6.66 (1H, m), 6.62 (1H, dd), 4.32-4.24 (1H, m), 3.14-3.06 (1H, m), 2.98-2.86 (1H, m), 1.46 (9H, s).

### Preparation 35: 3-Amino-7-fluoro-3,4-dihydro-1H-quinolin-2-one hydrochloride

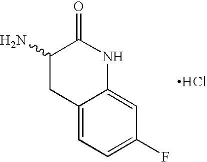

To a solution of (7-fluoro-2-oxo-1,2,3,4-tetrahydroquinolin-3-yl) carbamic acid tert-butyl ester (Preparation 34, 840 mg, 3.0 mmol) in methanol (20 mL) was added a solution of 4M hydrogen chloride in dioxane (1.5 mL, 6.0 mmol) and the reaction stirred for 3 h. Solvent was concentrated in vacuo, then the residue was dissolved in water (20 mL) and washed with EtOAc (20 mL). The aqueous solvent was removed in vacuo to afford the title compound. δ_{H} (CD₃OD): 7.23 (1H, dd), 6.80-6.70 (1H, m), 6.65 (1H, dd), 4.17 (1H, dd), 3.32-3.19 (1H, m), 3.13-2.98 (1H, m).

### Preparation 36: (3-Amino-7-fluoro-2-oxo-3,4-dihydro-2H-quinolin-1-yl)acetic acid methyl ester

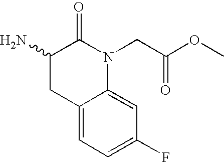

3-Amino-7-fluoro-3,4-dihydro-1H-quinolin-2-one hydrochloride (Preparation 35) was alkylated according to Preparation 26. Purification by column chromatography (SiO₂, 92:8 CH₂Cl₂, MeOH) afforded the title compound. δ_{H} (d₆ DMSO): 7.31-7.22 (1H, m), 6.93 (1H, dd), 6.88-6.78 (1H, m), 4.82-4.48 (2H, m), 3.66 (3H, s), 3.46 (1H, dd), 2.98 (1H, dd), 2.77-2.65 (1H, m).

### Preparation 37: 3-Amino-1-[2-tert-butyldimethylsilyloxy)ethyl]-7-fluoro-3,4-dihydro-1H-quinolin-2-one

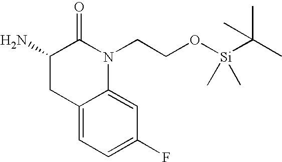

3-Amino-7-fluoro-3,4-dihydro-1H-quinolin-2-one hydrochloride (Preparation 35) was alkylated according to Preparation 28 to give the title compound. δ_{H} (CDCl₃): 7.12-7.04 (2H, m), 6.72-6.64 (1H, m), 4.18-4.09 (1H, m), 3.92-3.79 (3H, m), 3.52 (1H, dd), 3.0 (1H, dd), 2.81-2.70 (1H, m), 0.83 (9H, s), 0.05-0.00 (6H, m).

### Preparation 38: 5-Fluoro-2-nitrobenzaldehyde

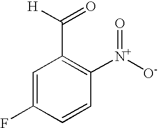

5-Fluoro-2-nitrotoluene was reacted in the same way as 4-fluoro-2-nitrotoluene according to Preparation 32 to afford the title compound. δ_{H} (CDCl₃): 10.43 (1H, d), 8.20 (1H, dd), 7.61 (1H, dd), 7.45-7.37 (11H, m).

### Preparation 39: 2-tert-Butoxycarbonylamino-3-(5-fluoro-2-nitrophenyl)acrylic acid methyl ester

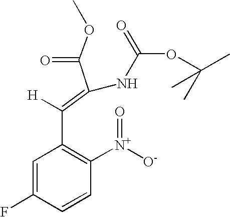

5-Fluoro-2-nitrobenzaldehyde (Preparation 38) was reacted according to Preparation 33 to afford the title compound. δ_{H} (CDCl₃): 8.16 (1H, dd), 7.50 (1H, s), 7.22 (1H, dd), 7.11-7.05 (1H, m), 6.52 (1H, brs), 3.87 (3H, s), 1.27 (9H, s).

### Preparation 40: (6-Fluoro-2-oxo-1,2,3,4-tetrahydroquinolin-3-yl) carbamic acid tert-butyl ester

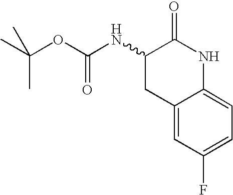

2-tert-Butoxycarbonylamino-3-(5-fluoro-2-nitrophenyl)acrylic acid methyl ester (Preparation 39) was reacted according to Preparation 15 to afford the title compound. δ_{H} (CDCl₃): 8.43 (1H, br s), 6.94-6.87 (2H, m), 6.80-6.74 (1H, m), 5.6 (1H, br s), 4.38-4.25 (1H, m), 3.53-3.39 (1H, m), 2.90-2.75 (1H, m), 1.47 (9H, s).

### Preparation 41: 3-Amino-6-fluoro-3,4-dihydro-1H-quinolin-2-one hydrochloride

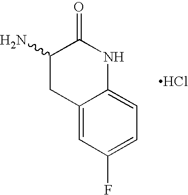

(6-Fluoro-2-oxo-1,2,3,4-tetrahydroquinolin-3-yl) carbamic acid tert-butyl ester (Preparation 40) was deprotected according to Preparation 35 to afford the title compound. δ_{H} (CD₃OD): 7.08-6.89 (3H, m), 4.22-4.13 (1H, m), 3.35-3.23 (1H, m), 3.19-3.08 (1H, m).

### Preparation 42: 5-Chloro-1H-pyrrolo[2,3-c]pyridine-2-carboxylic acid{1-[2-tert-butyldimethylsilyloxy)ethyl]-7-fluoro-2-oxo-1,2,3,4-tetrahydroquinolin-3-yl}amide

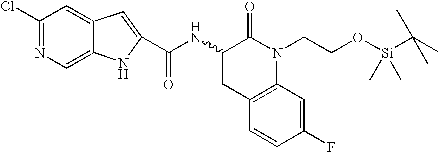

The title compound was prepared as described in Example 12 from 5-chloro-1H-pyrrolo[2,3-c]pyridine-2-carboxylic acid (Preparation 18) and the appropriate amine. m/z (ES⁺)=519.09[M+H]+; RT 4.28 min.

### Preparation 43 and 44

The procedure described in Example 48 was used to prepare the compounds of Preparation 43 and 44 from 5-chloro-1H-pyrrolo[2,3-c]pyridine-2-carboxylic acid (Preparation 3) and the appropriate amine.

| | | | RT | |
|---|---|---|---|---|
| Prep | Structure | Amine | (min) | m/z (ES⁺) |
| 43 | 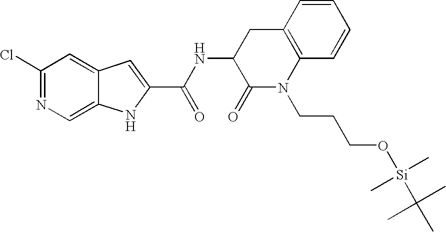 | WO03/074532 | 4.39 | 513[M + H]⁺ |
| 44 | 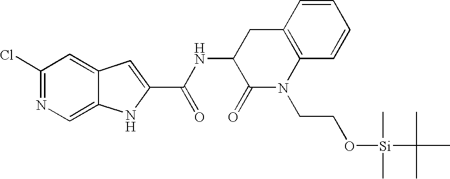 | Preparation 16 | 4.42 | 499[M + H]⁺ |

### Preparation 45: 4-Chloro-2-(2,2-dimethylpropionylamino)benzoic acid methyl ester

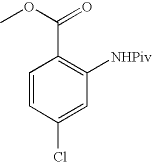

To a solution of commercially available 2-amino-4-chloro-benzoic acid methyl ester (LANCASTER, 3.0 g, 16.2 mmol) in dry DCM (50 mL) was added DIPEA (5.1 mL, 29.3 mmol), pivaloyl chloride (3.6 mL, 29.2 mmol) and 4-dimethylaminopyridine (50 mg, 0.41 mmol). The mixture was stirred for 2.5 h, then diluted with EtOAc (300 mL) and washed with diluted hydrochloric acid (1N, 100 mL) and brine (100 mL) before being dried (MgSO₄) and concentrated to give a solid residue. Recrystallisation from methanol gave the title compound as colourless needles. δ_{H} (CDCl₃): 1.35 (9H, s), 3.93 (3H, s), 7.04 (1H, d), 7.96 (1H, d), 8.91 (1H, s), 11.35 (1H, br s); m/z (ES⁻)=268.08 [M−H]⁻; RT=4.06 min.

### Preparation 46: N-(5-Chloro-2-hydroxymethylphenyl)-2,2-dimethylpropionamide

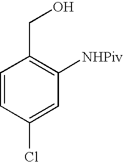

To a solution of 4-chloro-2-(2,2-dimethylpropionylamino)benzoic acid methyl ester (Preparation 45, 1.83 g, 6.78 mmol) in ethanol (100 mL) and water (10 mL) was added sodium borohydride (530 mg, 14.0 mmol) and the mixture stirred for 3 h at rt. The solution was acidified with dilute hydrochloric acid (pH 2-3) and diluted with water (200 mL) before being concentrated to half the original volume. The aqueous layer extracted with EtOAc (3×50 mL) and the combined extracts were dried (MgSO₄) and concentrated to an oily residue. Purification by flash chromatography on silica gel (eluent: hexane/EtOAc: 2/1) gave the title compound as colourless oil. δ_{H} (CDCl₃): 1.29 (9H, s), 2.80 (1H, br s) 4.65 (2H, s), 7.00 (1H, dd), 7.04 (1H, d), 8.19 (1H, d), 9.09 (1H, br s); m/z (ES⁻)=240.06 [M−H]⁻; RT=3.19 min.

### Preparation 47: N-(5-Chloro-2-formylphenyl)-2,2-dimethylpropionamide

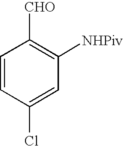

To a solution of N-(5-chloro-2-hydroxymethylphenyl)-2,2-dimethylpropionamide (Preparation 46, 970 mg, 4.01 mmol) in dry DCM (40 mL) was added Dess-Martin periodinane (1.80 g, 4.24 mmol). After stirring for 2 h at rt alkaline sodium thiosulfate solution was added (27 g Na₂SO₃ dissolved in 100 mL saturated NaHCO₃ solution) and the emulsion was vigorously stirred for additional 20 min. The layers were separated and the aqueous layer extracted with ethyl acetate (2×50 mL). Washing of the combined extracts with saturated sodium hydrogen carbonate solution (50 mL) and brine (50 mL) gave a solution, which was concentrated after drying (MgSO₄). Purification of the residue by flash chromatography on silica gel (eluent: hexane/EtOAc:5/1) gave the title compound as colourless oil. δ_{H} (CDCl₃): 1.36 (9H, s), 7.19 (1H, dd), 7.60 (1H, d), 8.91 (1H, d), 9.90 (1H, s), 11.45 (1H, br s); m/z (ES⁺)=240.06 [M+H]⁺; RT=3.79 min.

### Preparation 48: (E,Z)-2-tert-Butoxycarbonylamino-3-[4-chloro-2-(2,2-dimethylpropionylamino)phenyl]acrylic acid methyl ester

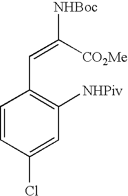

To a solution of (+/−)-Boc-α-phosphonoglycine trimethyl ester (1.10 g, 3.70 mmol) in dry THF (10 mL) at −78° C. was added 1,1,3,3-tetramethylguanidine (0.45 mL, 3.59 mmol) and the mixture was stirred in the cold for 20 min. A solution of N-(5-chloro-2-formylphenyl)-2,2-dimethylpropionamide (Preparation 47, 740 mg, 3.09 mmol) in dry THF (10 mL) was added and the resulting mixture was allowed to warm up to rt and stirred for additional 12 h. Distribution between EtOAc (200 mL) and water (100 mL) followed by separation of the organic layer gave after drying (MgSO₄) and concentration an oily residue. Purification of the residue by flash chromatography on silica gel (eluent: hexane/EtOAc:2/1) gave the title compound(s) as a colourless oil(s). δ_{H} (CDCl₃)—major product: 1.33 (9H, s), 1.34 (9H, s), 3.91 (3H, s), 6.43 (1H, br s), 7.08 (1H, s), 7.11 (1H, dd), 7.22 (1H, d), 7.61 (1H, br s), 8.11 (1H, d)—minor product: 1.31 (9H, s), 1.54 (9H, s), 3.63 (3H, s), 6.99 (1H, brs), 7.06 (2H, m), 7.54 (1H, m), 7.61 (1H, br s), 8.34 (1H, m); m/z (ES)=409.14 [M−H]⁻; RT=3.72 min.

### Preparation 49: 2-tert-Butoxycarbonylamino-3-[4-chloro-2-(2,2-dimethylpropionylamino)phenyl]propionic acid methyl ester

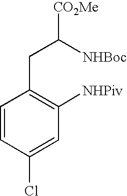

To a solution of (E,Z)-2-tert-butoxycarbonylamino-3-[4-chloro-2-(2,2-dimethyl propionylamino)phenyl]acrylic acid methyl ester (Preparation 48, 502 mg, 1.22 mmol) in methanol (10 mL) were added magnesium turnings (60 mg, 2.47 mmol) and the mixture stirred at rt for 12 h. Toluene (200 mL) was added and the resulting organic layer was washed with diluted hydrochloric acid (0.1N, 50 mL) and brine (50 mL) before dried (MgSO₄) and concentrated. The resulting oil was used in the next step without further purification. δ_{H} (CDCl₃): 1.38 (9H, s), 1.43 (9H, s), 2.96-3.10 (2H, m), 3.74 (3H, s), 4.56 (1H, m), 5.19 (1H, br d), 7.06 (1H, dd), 7.11 (1H, dd), 7.84 (1H, m), 8.06 (1H, br s); m/z (ES⁻)=411.18 [M−H]⁻; RT=3.92 min.

### Preparation 50: (3-Amino-7-chloro-2-oxo-3,4-dihydro-2H-quinolin-1-yl)acetic acid methyl ester

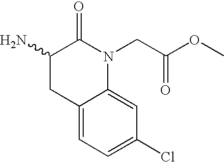

To a vigorously stirred solution of 3-amino-7-chloro-3,4-dihydro-1H-quinolin-2-one (Preparation 19, 630 mg, 3.24 mmol) in DMF (20 mL) at 0° C. was added sodium hydride dispersion (146 mg, 3.65 mmol, 60%). After 30 min methyl bromoacetate (0.31 mL, 3.27 mmol) was added, the cooling bath was removed and the resulting mixture stirred for further 12 h before added into saturated sodium hydrogencarbonate solution (150 mL). Extraction with EtOAc (3×50 mL) and washing of the combined extracts with brine (50 mL) gave after drying (MgSO₄) and concentration in vacuo an oily residue. Purification of the residue by flash chromatography on silica gel (eluent: DCM then DCM/methanol: 19/1 then DCM/methanol: 9/1) gave the title compound as colourless oil. δ_{H} (CDCl₃): 1.80 (2H, br s), 2.87 (1H, dd), 3.08 (1H, dd), 3.63 (1H, m), 3.79 (3H, s), 4.43 (1H, d), 4.86 (1H, d), 6.74 (1H, d), 7.02 (1H, dd), 7.15 (1H, d); m/z (ES⁺)=269.04 [M+H]⁺; RT=2.04 min.

### Preparation 51: 3-Amino-1-[2-(tert-butyldimethylsilanyloxy)ethyl]-7-chloro-3,4-dihydro-1H-quinolin-2-one

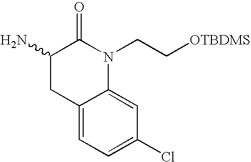

To a vigorously stirred solution of 3-amino-7-chloro-3,4-dihydro-1H-quinolin-2-one (Preparation 19, 149 mg, 0.76 mmol) in DMF (5 mL) at 0° C. was added sodium hydride dispersion (38 mg, 0.95 mmol, 60%). After 1 h (2-bromoethoxy)-tert-butyldimethylsilane (165 μL, 0.77 mmol) was added, the cooling bath was removed and the resulting mixture was stirred for further 12 h before being added into saturated sodium hydrogen carbonate solution (100 mL). Extraction with EtOAc (3×50 mL) and washing of the combined extracts with brine (50 mL) gave after drying (MgSO₄) and concentration in vacuo an oily residue. Purification of the residue by flash chromatography on silica gel (eluent: DCM/methanol: 19/1) gave the title compound as a colourless oil. δ_{H} (CDCl₃): 0.00, 0.03 (6H, 2×s), 0.85 (9H, s), 1.73 (2H, br s), 2.79 (1H, dd), 3.03 (1H, dd), 3.54 (1H, dd), 3.86-3.93 (3H, m), 4.19 (1H, m), 6.98 (1H, dd), 7.09 (1H, d), 7.39 (1H, d); m/z (ES⁺)=355.15 [M+H]⁺; RT=3.27 min.

### Preparation 52: 2-tert-Butoxycarbonylamino-3-[2-(2,2-dimethylpropionylamino)-pyridin-3-yl]acrylic acid methyl ester

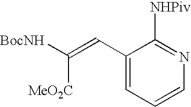

To a solution of (+/−)-Boc-α-phosphonoglycine trimethyl ester (1.52 g, 5.11 mmol) in dry THF (20 mL) at −78° C. was added 1,1,3,3-tetramethylguanidine (0.60 mL, 4.78 mmol) and the mixture was stirred in the cold for 20 min. A solution of commercially available N-(3-formylpyridin-2-yl)-2,2-dimethylpropionamide (SPECS and BioSPECS, 960 mg, 4.65 mmol) in dry THF (20 mL) was added dropwise and the resulting mixture allowed to warm up to rt and stirred for additional 12 h before being poured into water (200 mL). Extraction with EtOAc (3×75 mL) and washing of the combined extracts with brine (50 mL) gave after drying (MgSO₄) and concentration in vacuo an oily residue. Purification of the residue by flash chromatography on silica gel (eluent: hexane/EtOAc: 1/3) gave the title compound as a colourless oil. δ_{H} (CDCl₃): 1.33 (9H, s), 1.37 (9H, s), 3.84 (3H, s), 6.37 (1H, br s), 7.06 (1H, s), 7.16 (1H, dd), 7.80-7.83 (2H, m), 8.38 (1H, d); m/z (ES⁺)=377.97 [M+H]⁺; RT=3.36 min.

### Preparation 53: 2-tert-Butoxycarbonylamino-3-[2-(2,2-dimethylpropionylamino)pyridin-3-yl]propionic acid methyl ester

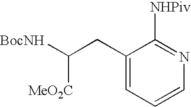

Palladium-on-carbon (266 mg, 10 wt %) was added to a solution of 2-tert-butoxycarbonylamino-3-[2-(2,2-dimethylpropionylamino)pyridin-3-yl]acrylic acid methyl ester (Preparation 52, 1.40 g, 3.71 mmol) in ethanol (50 mL) and the mixture stirred under an atmosphere of hydrogen for 12 h. After filtration through celite and repeated washing of the catalyst with methanol, the filtrate and washings were combined and concentrated in vacuo to give the title compound as colourless oil. δ_{H} (CDCl₃): 1.34 (9H, s), 1.37 (9H, s), 2.83 (1H, dd), 3.22 (1H, dd), 3.71 (3H, s), 4.62 (1H, m), 5.32 (1H, br d), 7.14 (1H, dd), 7.58 (1H, d), 7.98 (1H, br s), 8.34 (1H, d); m/z (ES⁺)=380.11 [M+H]⁺; RT=3.24 min.

### Preparation 54: 3-Amino-3,4-dihydro-1H-[1,8]naphthyridin-2-one dihydrochloride

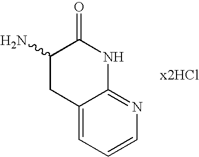

2-tert-Butoxycarbonylamino-3-[2-(2,2-dimethylpropionylamino)pyridin-3-yl]propionic acid methyl ester (Preparation 53, 1.30 g, 3.43 mmol) was dissolved in dilute hydrochloric acid (2N, 50 mL) and the solution heated under reflux for 72 h. After cooling to rt and concentration in vacuo the residue was taken up in water (200 mL). The aqueous layer was washed with ethyl acetate (2×50 mL) and concentrated. Addition of methanol (˜10 mL) led to the precipitation of the title compound, which was obtained as an off-white solid after filtration. δ_{H} (D₂O): 3.36 (1H, dd), 3.59 (1H, dd), 4.55 (1H, dd), 7.43 (1H, dd), 8.15 (1H, d), 8.27 (1H, d); m/z (ES⁺)=164.05 [M−2HCl+H]⁺; RT=0.31 min.

### Preparation 55: 4-[N-tert-Butyloxycarbonyl)amino]-3-pyridine-carboxaldehyde

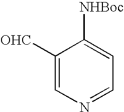

The title compound was synthesized via a known method from 4-[N-(tert-butyloxycarbonyl)amino]pyridine (M. C. Venuti et al., J. Med. Chem., 1988, 31, 2136-2145. δ_{H} (CDCl₃): 1.55 (9H, s), 8.34 (1H, d), 8.59 (1H, d), 8.76 (1H, s), 9.98 (1H, s), 10.43 (1H, br s); m/z (ES⁺)=223.01 [M+H]⁺; RT=3.01 min.

### Preparation 56: 2-tert-Butoxycarbonylamino-3-(4-tert-butoxycarbonylaminopyridin-3-yl)acrylic acid methyl ester

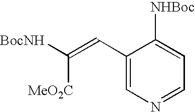

To a solution of (+/−)-Boc-α-phosphonoglycine trimethyl ester (1.50 g, 5.05 mmol) in dry THF (20 mL) at −78° C. was added 1,1,3,3-tetramethylguanidine (0.60 mL, 4.78 mmol) and the mixture was stirred in the cold for 20 min. A solution of 4-[N-(tert-butyloxycarbonyl)amino]-3-pyridinecarboxaldehyde (Preparation 55, 1.0 g, 4.50 mmol) in dry THF (10 mL) was added slowly and the resulting mixture allowed to warm up to rt and stirred for additional 12 h before being poured into water (200 mL). Extraction with EtOAc (3×75 mL) and washing of the combined extracts with brine (50 mL) gave after drying (MgSO₄) and concentration in vacuo an oily residue. Purification of the residue by flash chromatography on silica gel (eluent: toluene/acetone: 2/1) gave the title compound as colourless oil. δ_{H} (CDCl₃): 1.31 (9H, s), 1.53 (9H, s), 3.91 (3H, s), 6.61, 6.76 (2H, 2×br s), 7.05 (1H, s), 8.06 (1H, d), 8.37-8.39 (2H, m); m/z (ES⁺)=394.13 [M+H]⁺; RT=2.81 min.

### Preparation 57: 2-tert-Butoxycarbonylamino-3-(4-tert-butoxycarbonylaminopyridin-3-yl)propionic acid methyl ester

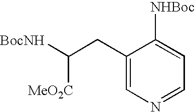

Palladium-on-carbon (225 mg, 10 wt %) was added to a solution of 2-tert-butoxycarbonylamino-3-(4-tert-butoxycarbonylaminopyridin-3-yl)acrylic acid methyl ester (Preparation 56, 1.02 g, 2.59 mmol) in ethanol (40 mL) and the mixture stirred under an atmosphere of hydrogen for 12 h. After filtration through celite and repeated washing of the catalyst with methanol, the filtrate and washings were combined and concentrated in vacuo to give the title compound as colourless oil. δ_{H} (CDCl₃): 1.50 (9H, s), 1.56 (9H, s), 2.94 (1H, dd), 3.16 (1H, m), 3.74 (3H, s), 4.30 (1H, m), 5.61 (1H, br d), 8.13-8.16 (2H, m), 8.37 (1H, d), 8.54 (1H, br s); m/z (ES⁺)=396.15 [M+H]⁺; RT=2.95 min.

### Preparation 58: 3-Amino-3,4-dihydro-1H-[1,6]naphthyridin-2-one dihydrochloride

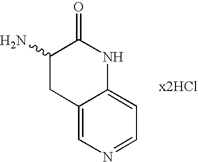

2-tert-Butoxycarbonylamino-3-(4-tert-butoxycarbonylaminopyridin-3-yl)propionic acid methyl ester (Preparation 57, 830 mg, 2.10 mmol) was dissolved in dilute hydrochloric acid (2N, 50 mL) and the solution was heated under reflux for 2 h. After cooling to rt and concentration in vacuo the residue was taken up in water (200 mL). The aqueous layer was washed with ethyl acetate (50 mL) and diethyl ether (50 mL) and then concentrated again to give the title compound as off-white solid. δ_{H} (D₂O): 3.40 (1H, dd), 3.69 (1H, m), 4.60 (1H, m), 7.44 (1H, m), 8.53 (1H, m), 8.61 (1H, m); m/z (ES⁺)=164.03 [M−2HCl+H]⁺; RT=0.22 min.

### Preparation 59: (3-Amino-2-oxo-3,4-dihydro-2H-[1,6]naphthyridin-1-yl)acetic acid methyl ester dihydrochloride

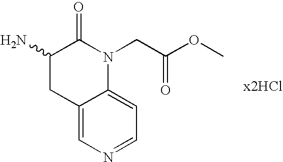

To a vigorously stirred solution of 3-amino-3,4-dihydro-1H-[1,6]naphthyridin-2-one dihydrochloride (Preparation 58, 203 mg, 0.86 mmol) in DMF (10 mL) at 0° C. was added sodium hydride dispersion (19 mg, 2.98 mmol, 60%). After 1.5 h methyl bromoacetate (80 μL, 0.85 mmol) was added, the cooling bath was removed and the resulting mixture was stirred for further 12 h before added into water (100 mL). The solution was made acidic (pH 2-3) with dilute hydrochloric acid (1N) and washed with ethyl acetate (30 mL) After concentration in vacuo the title compound was obtained as an oil, which was used in the next step without further purification. δ_{H} (D₂O): 3.53 (1H, dd), 3.75 (1H, dd), 4.74 (1H, m), 4.92 (1H, d), 5.14 (1H, d), 7.66 (1H, d), 8.70 (1H, d), 8.75 (1H, s); m/z (ES⁺)=236.03 [M−2HCl+H]⁺; RT=0.42 min.

### Preparation 60: (3-Amino-2-oxo-3,4-dihydro-2H-[1,5]naphthyridin-1-yl)acetic acid methyl ester

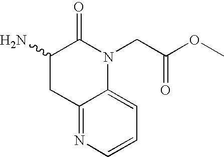

To a vigorously stirred solution of 3-amino-3,4-dihydro-1H-[1,5]naphthyridin-2-one dihydrochloride (WO 03/074532, 202 mg, 0.86 mmol) in DMF (10 mL) at 0° C. was added sodium hydride dispersion (120 mg, 3.00 mmol, 60%). After 1.5 h methyl bromoacetate (80 μL, 0.85 mmol) was added, the cooling bath was removed and the resulting mixture was stirred for further 12 h before added into saturated sodium hydrogencarbonate solution (150 mL).

Extraction with EtOAc (3×50 mL) and washing of the combined extracts with brine (50 mL) gave after drying MgSO₄) and concentration in vacuo an oily residue. Purification of the residue by flash chromatography on silica gel (eluent: DCM/methanol: 85/15) gave the title compound as colourless oil. δ_{H} (d₄ MeOH): 3.17 (1H, dd), 3.32 (1H, dd), 3.76 (3H, s), 3.79 (1H, dd), 4.60 (1H, d), 4.89 (1H, d), 7.34 (1H, dd) 7.41 (1H, d), 8.18 (1H, d); m/z (ES⁺)=236.05 [M+H]⁺; RT=0.53 min.

### Preparation 61: 3-Amino-1-[2-tert-butyldimethylsilanyloxy)ethyl]-3,4-dihydro-1H-[1,5]naphthyridin-2-one

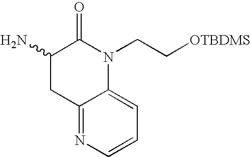

To a vigorously stirred solution of 3-amino-3,4-dihydro-1H-[1,5]naphthyridin-2-one dihydrochloride (WO 03/074532, 200 mg, 0.85 mmol) in DMF (10 mL) at 0° C. was added sodium hydride dispersion (114 mg, 2.85 mmol, 60%). After 45 min (2-bromoethoxy)-tert-butyldimethylsilane (185 μL, 0.86 mmol) was added, the cooling bath was removed and the resulting mixture was stirred for further 12 h before added into saturated sodium hydrogencarbonate solution (150 mL). Extraction with ethyl acetate (3×50 mL) and washing of the combined extracts with brine (50 mL) gave after drying (MgSO₄) and concentration in vacuo an oily residue. Purification of the residue by flash chromatography on silica gel (eluent: DCM/methanol: 9/1) gave the title compound as colourless oil. δ_{H} (d₄ MeOH): 0.00, 0.02 (6H, 2×s), 0.84 (9H, s), 3.09 (1H, dd), 3.30 (1H, dd), 3.74 (1H, dd), 3.89-4.23 (4H, 3×m), 7.36 (1H, dd), 7.82 (1H, d), 8.17 (11, d); m/z (ES⁺)=322.13 [M+H]⁺; RT=2.74 min.

### Preparation 62: 3-Amino-3,4-dihydro-1H-[1,7]naphthyridin-2-one dihydrochloride

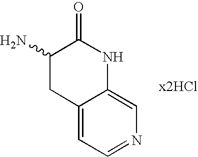

To a solution of tert-butyl(2-oxo-1,2,3,4-tetrahydro[1,7]naphthyridin-3-yl)carbamate (WO 03/074532, 400 mg, 1.52 mmol) in DCM (20 mL) was added hydrochloric acid (3 mL, 4N in dioxane). After stirring for 4 h at rt the solution was concentrated in vacuo before the residue was taken up in water (150 mL). The aqueous layer was washed with EtOAc (2×50 mL) and concentrated. Addition of methanol (˜5 mL) led to the precipitation of the title compound, which was obtained as a white solid after filtration. δ_{H} (D₂O): 3.56 (1H, dd), 3.75 (1H, dd), 4.55 (1H, dd), 7.95 (1H, d), 8.41 (1H, s), 8.46 (1H, d); m/z (ES⁺)=164.05 [M−2HCl+H]⁺; RT=0.21 min.

### Preparation 63: 5-Chloro-1H-pyrrolo[2,3-c]pyridine-2-carboxylic acid {1-[2-(tert-butyldimethyl-silanyloxy)ethyl]-2-oxo-1,2,3,4-tetrahydro-[1,5]naphthyridin-3-yl}amide

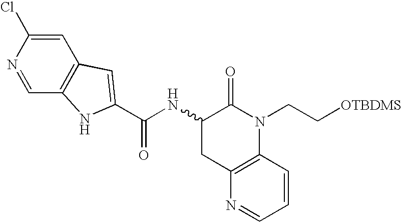

The title compound was prepared according to the method of Example 69 from chloro-1H-pyrrolo[2,3-c]pyridine-2-carboxylic acid (Preparation 3) and the appropriate amine: m/z (ES⁺)=500.11 [M+H]⁺; RT=3.98 min.

### Preparation 64: 5-Chloro-1H-pyrrolo[2,3-]pyridine-2-carboxylic acid {1-[2-(tert-butyldimethyl silanyloxy)ethyl]-7-chloro-2-oxo-1,2,3,4-tetrahydroquinolin-3-yl}amide

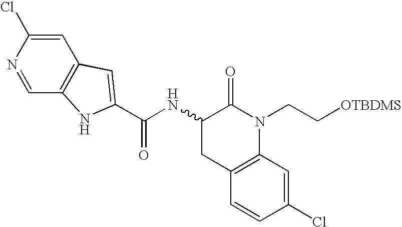

To a solution of 5-chloro-1H-pyrrolo[2,3-c]pyridine-2-carboxylic acid (Preparation 16, 115 mg, 0.59 mmol), EDCI (132 mg, 0.69 mmol) and HOBt monohydrate (89 mg, 0.58 mmol) in DMF (10 mL) was added 3-amino-1-[2-(tert-butyldimethylsilanyloxy)ethyl]-7-chloro-3,4-dihydro-1H-quinolin-2-one (Preparation 7, 200 mg, 0.56 mmol) and DIPEA (220 μL, 1.26 mmol). The resulting solution was stirred for 12 h at rt before the reaction mixture was partitioned between EtOAc (50 mL) and water/brine (100 mL, 1:1). The layers were separated and the aqueous phase was extracted with EtOAc (3×50 mL), then the combined organics were washed with dilute HCl solution (1M, 50 mL), dilute NaOH solution (1M, 50 mL) and brine (50 mL). The organic phase was dried (MgSO₄), filtered and concentrated in vacuo. Purification of the residue by flash chromatography on silica gel (eluent: hexane/EtOAc: 1/2) gave the title compound as a colourless solid. TLC (hexane/EtOAc: 1/3): R_{f} 0.60; δ_{H} (d₆ DMSO): 0.00, 0.01 (6H, 2×s), 0.83 (9H, s), 3.14-3.21 (2H, m), 3.84-3.98 (3H, 2×m), 4.20 (1H, m), 4.78 (1H, ddd), 7.15 (1H, dd), 7.30 (1H, s), 7.34 (1H, d), 7.49 (1H, d), 7.83 (1H, s), 8.63 (1H, s), 9.15 (1H, d), 12.41 (1H, br s); m/z (ES⁺)=533.19 [M+H]⁺; RT=4.77 min.

### Example 1

### 6-Chloro-1H-pyrrolo[2,3-b]pyridine-2-carboxylic acid (2-oxo-1,2,3,4-tetrahydroquinolin-3-yl)amide

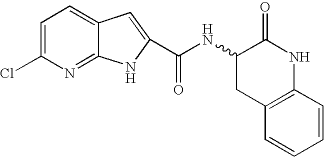

To a solution of 3-amino-3,4-dihydro-1H-quinolin-2-one (Preparation 15, 27 mg, 0.17 mmol) in DMF (4 mL) was added 6-chloro-1H-pyrrolo[2,3b]pyridine-2-carboxylic acid (Preparation 12, 30 mg, 0.15 mmol), HOBt (26 mg, 0.17 mmol) and DIPEA (66 μL, 0.38 mmol) and the reaction stirred for 5 min. EDCI (35 mg, 0.18 mmol) was added and the reaction stirred at rt for 16 h. Solvent was removed in vacuo and the residue partitioned between EtOAc (30 mL) and water (30 mL). Organics were washed with water (30 mL), NaHCO₃ solution (2×25 mL) then brine (2×25 mL) before being dried (MgSO₄) and concentrated in vacuo. Purification by Prep HPLC afforded the title compound. δ_{H} (d₆ DMSO): 8.20 (1H, d), 7.29-7.17 (4H, m), 7.00-6.89 (2H, m), 4.80-4.70 (1H, m), 3.21-3.06 (2H, m); m/z (ES⁺)=341.09 [M+H]⁺; RT=3.33 min.

### Example 2

### 6-Chloro-1H-pyrrolo[2,3-b]pyridine-2-carboxylic acid (7-chloro-2-oxo-1,2,3,4-tetrahydroquinolin-3-yl)amide

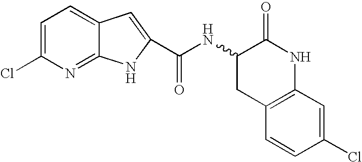

The title compound was prepared according to Example 1 using 3-amino-7-chloro-3,4-dihydro-1H-quinolin-2-one (Preparation 19) instead of 3-amino-3,4-dihydro-1H-quinolin-2-one. δ_{H} (d₆ DMSO): 8.19 (1H, d), 7.30-7.24 (3H, m), 7.20 (1H, d), 6.94 (1H, s), 4.81-4.71 (1H, m), 3.16-3.09 (2H, m); m/z (ES⁺)=375.05 [M+H]⁺; RT=3.46 min.

### Example 3

### (R)-5-Chloro-1H-pyrrolo[2,3-c]pyridine-2-carboxylic acid (2-oxo-1,2,3,4-tetrahydroquinolin-3-yl)amide

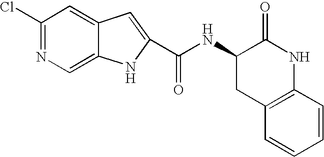

To a suspension of 3-(R)-amino-3,4-dihydro-1H-quinolin-2-one hydrochloride (Preparation 24, 67 mg, 0.34 mmol) in DMF (5 mL) under argon was added DIPEA (186 μL, 1.07 mmol), 5-chloro-1H-pyrrolo[2,3-c]pyridine-2-carboxylic acid (Preparation 3, 60 mg, 0.31 mmol) and HOBt (51 mg, 0.34 mmol) and the reaction stirred for 5 min. EDCI (76 mg, 0.5 mmol) was added and the reaction stirred for 16 h at rt. Solvent was removed in vacuo and the residue partitioned between EtOAc (5 mL) and water (40 mL). Organics were washed with NaHCO₃ solution (2×15 mL) then brine (15 mL) before being dried (MgSO₄) and solvent removed in vacuo. Purification by crystallisation from methanol afforded the title compound. δ_{H} (d₆ DMSO): 8.61 (1H, s), 7.80 (1H, s), 7.29-7.17 (3H, m), 7.00-6.90 (2H, m), 4.82-4.73 (1H, m), 3.22-3.06 (2H, m); m/z (ES⁺)=341.03 [M+H]⁺; RT=3.24 min.

### Example 4

### (S)-5-Chloro-1H-pyrrolo[2,3-c]pyridine-2-carboxylic acid (2-oxo-1,2,3,4-tetrahydroquinolin-3-yl)amide

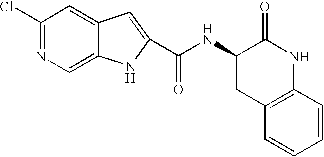

The title compound was prepared from 3-(S)-amino-3,4-dihydro-1H-quinolin-2-one hydrochloride (Preparation 25) according to Example 3. δ_{H} (d₆ DMSO): 8.61 (1H, s), 7.80 (1H, s), 7.29-7.17 (3H, m), 7.00-6.90 (2H, m), 4.82-4.73 (1H, m), 3.22-3.06 (2H, m); m/z (ES⁺)=341.02 [M+H]⁺; RT=3.20 min.

### Example 5

### {3-(R)-[(5-Chloro-1H-pyrrolo[2,3-c]pyridine-2-carbonyl)amino]-2-oxo-3,4-dihydro-2H-quinolin-1-yl}acetic acid methyl ester

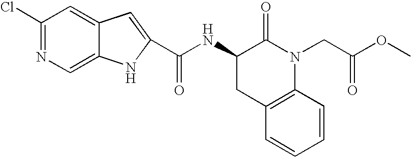

To a solution of 5-chloro-1H-pyrrolo[2,3-c]pyridine-2-carboxylic acid (Preparation 3, 30 mg, 0.15 mmol) in DMF (5 mL) was added (3-(R)-amino-2-oxo-3,4-dihydro-2H-quinolin-1-yl)-acetic acid methyl ester (Preparation 26, 36 mg, 0.15 mmol), HOBt (26 mg, 0.17 mmol) and DIPEA (67 μL, 0.38 mmol) and the reaction stirred for 5 min. EDCI (35 mg, 0.18 mmol) was added and the reaction stirred for 16 h at rt. Solvent was concentrated in vacuo and the residue partitioned between EtOAc (30 mL) and water (30 mL). Organics were washed with 1M NaOH (2×20 mL) and brine (20 mL) then dried (MgSO₄) and the solvent removed in vacuo. Trituration with methanol afforded the title compound. δ_{H} (d₆ DMSO): 8.61 (1H, s), 7.80 (1H, s), 7.39-7.26 (3H, m), 7.14-7.06 (2H, m), 4.91-4.77 (2H, m), 7.10-7.04 (1H, m), 3.70 (3H, s), 3.34-3.06 (2H, m); m/z (ES⁺)=413.05 [M+H]⁺; RT=3.31 min.

### Example 6

### {3-(S)-[(5-Chloro-1H-pyrrolo[2,3-]pyridine-2-carbonyl)amino]-2-oxo-3,4-dihydro-2H-quinolin-1-yl}acetic acid methyl ester

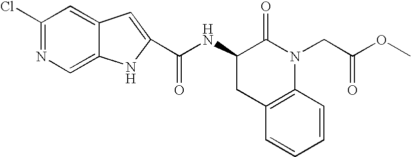

The title compound was prepared from (3-(S)-Amino-2-oxo-3,4-dihydro-2H-quinolin-1-yl)-acetic acid methyl ester (Preparation 27) according to Example 5. δ_{H} (d₆ DMSO): 8.61 (1H, s), 7.80 (1H, s), 7.39-7.26 (3H, m), 7.14-7.06 (2H, m), 4.91-4.77 (2H, m), 7.10-7.04 (1H, m), 3.70 (3H, s), 3.34-3.06 (2H, m); m/z (ES⁺)=413.04 [M+H]⁺; RT=3.34 min.

### Example 7

### {3-(R)-[(5-Chloro-1H-pyrrolo[2,3-c]pyridine-2-carbonyl)amino]-2-oxo-3,4-dihydro-2H-quinolin-1-yl}acetic acid

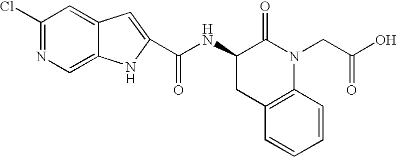

To a suspension of {3-(R)-[(5-chloro-1H-pyrrolo[2,3-c]pyridine-2-carbonyl)amino]-2-oxo-3,4-dihydro-2H-quinolin-1-yl}acetic acid methyl ester (Example 5, 15.2 mg, 0.04 mmol) in THF (2 mL) was added a 2M solution of LiOH (40 μL, 0.08 mmol) and the reaction stirred for 3 h. Solvent was concentrated in vacuo then the residue dissolved in water (15 mL). The aqueous phase was washed with EtOAc before being acidified to pH 2 with 2M HCl. Organics were extracted into EtOAc (20 mL) and the solvent removed in vacuo to afford the title compound. δ_{H} (CD₃OD): 8.63 (1H, s), 7.73 (1H, s), 7.39-7.30 (2H, m), 7.23 (1H, s), 7.14 (1H, m), 7.04 (1H, d), 5.03-4.89 (2H, m), 4.63 (1H, d), 3.37-3.20 (2H, m); m/z (ES⁺)=399.01 [M+H]⁺; RT=3.20 min.

### Example 8

### {3-(S)-[(5-Chloro-1H-pyrrolo[2,3-c]pyridine-2-carbonyl)amino]-2-oxo-3,4-dihydro-2H-quinolin-1-yl}acetic acid

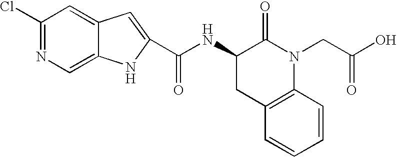

The title compound was prepared from {3-(S)-[(5-chloro-1H-pyrrolo[2,3-c]pyridine-2-carbonyl)amino]-2-oxo-3,4-dihydro-2H-quinolin-1-yl}acetic acid methyl ester (Example 6) according to Example 7. δ_{H} (CD₃OD): 8.50 (1H, s), 7.60 (1H, s), 7.23-7.20 (2H, m), 7.10 (1H, s), 7.01 (1H, m), 6.91 (1H, d), 4.89-4.77 (2H, m), 4.50 (1H, d), 3.23-3.08 (2H, m); m/z (ES⁺)=399.00 [M+H]⁺; RT=3.18 min.

### Example 9

### (R)-5-Chloro-1H-pyrrolo[2,3-c]pyridine-2-carboxylic acid[1-(2-hydroxyethyl)-2-oxo-1,2,3,4-tetrahydroquinolin-3-yl}amide

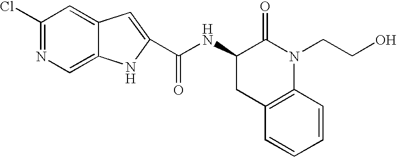

To a solution of (R)-5-chloro-1H-pyrrolo[2,3-c]pyridine-2-carboxylic acid {1-[2-(tert-butyldimethylsilyloxy)ethyl]-2-oxo-1,2,3,4-tetrahydroquinolin-3-yl}amide (Preparation 30, 120 mg, 0.24 mmol) in THF (5 mL) was added a solution of TBAF in THF (11.0M, 360 μL, 0.36 mmol) and the reaction stirred for 16 h at rt before concentrating the solvent in vacuo. Purification by column chromatography (SiO₂, 91:9 DCM/MeOH) afforded the title compound. δ_{H} (d₆ DMSO): 8.66 (1H, s), 7.80 (1H, s), 7.39-7.24 (4H, m), 7.09 (1H, m), 4.81-4.71 (1H, m), 4.11-3.89 (2H, br m), 3.70-3.57 (2H, m), 3.26-3.01 (2H, m); m/z (ES⁺)=385.09 [M+H]⁺; RT=3.04 min.

### Example 10

### (S)-5-Chloro-1H-pyrrolo[2,3-c]pyridine-2-carboxylic acid[1-(2-hydroxyethyl)-2-oxo-1,2,3,4-tetrahydroquinolin-3-yl}amide

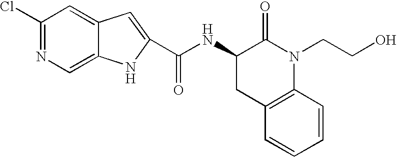

The title compound was prepared from (S)-5-chloro-1H-pyrrolo[2,3-c]pyridine-2-carboxylic acid {1-[2-(tert-butyldimethylsilyloxy)ethyl]-2-oxo-1,2,3,4-tetrahydroquinolin-3-yl}amide (Preparation 31) according to Example 9. δ_{H} (d₆ DMSO): 8.66 (1H, s), 7.80 (1H, s), 7.39-7.24 (4H, m), 7.09 (1H, m), 4.81-4.71 (1H, m), 4.11-3.89 (2H, br m), 3.70-3.57 (2H, m), 3.26-3.01 (2H, m); m/z (ES⁺)=385.06 [M+H]⁺; RT=3.02 min.

### Example 11

### 5-Chloro-1H-pyrrolo[2,3-c]pyridine-2-carboxylic acid (7-fluoro-2-oxo-1,2,3,4-tetrahydroquinolin-3-yl)amide

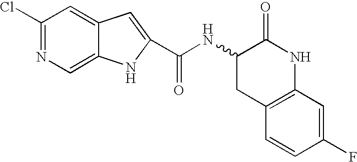

To a suspension of 3-amino-7-fluoro-3,4-dihydro-1H-quinolin-2-one hydrochloride (Preparation 35, 100 mg, 0.4 mmol) in anhydrous THF (5 mL) was added DMTMM (349 mg, 1.19 mmol) and 4-methyl morpholine (130 μL, 1.19 mmol), followed by 5-chloro-1H-pyrrolo[2,3-c]pyridine-2-carboxylic acid (Preparation 3, 71 mg, 0.36 mmol), and the reaction stirred at rt for 16 h. Solvent was concentrated in vacuo and the residue taken into EtOAc (50 mL). Organics were washed with water (30 mL), 1M HCl (30 mL) and brine (30 mL) before being dried (MgSO₄) and removing the solvent in vacuo. Trituration from methanol afforded the title compound. δ_{H} (d₆ DMSO): 12.35 (1H, s), 10.50 (1H, s), 9.07 (1H, d), 8.59 (1H, s), 7.78 (1H, s), 7.33-7.20 (2H, m), 6.83-6.64 (2H, m), 4.83-4.71 (1H, m), 3.11 (2H, d); m/z (ES⁺)=359.06 [M+H]⁺; RT=3.44 min.

### Example 12

### {3-[(5-Chloro-1H-pyrrolo[2,3-]pyridine-2-carbonyl)amino]-7-fluoro-2-oxo-3,4-dihydro-2H-quinolin-1-yl}acetic acid methyl ester

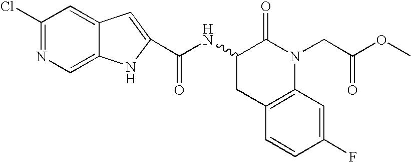

5-Chloro-1H-pyrrolo[2,3-c]pyridine-2-carboxylic acid (Preparation 3) was reacted with (3-amino-7-fluoro-2-oxo-3,4-dihydro-2H-quinolin-1-yl)acetic acid methyl ester (Preparation 36) according to Example 11. Purification by column chromatography (SiO₂, DCM/MeOH) afforded the title compound. δ_{H} (d₆ DMSO): 12.35 (1H, s), 9.19 (1H, d), 8.59 (1H, s), 7.79 (1H, s), 7.38-7.32 (1H, m), 7.26 (1H, s), 7.09-7.03 (1H, m), 6.95-6.88 (1H, m), 4.86-4.65 (3H, m), 3.68 (3H, s), 3.33-3.08 (2H, m); m/z (ES⁺)=431.03 [M+H]⁺; RT=3.38 min.

### Example 13

### 5-Chloro-1H-pyrrolo[2,3-c]pyridine-2-carboxylic acid(6-fluoro-2-oxo-1,2,3,4-tetrahydroquinolin-3-yl)amide

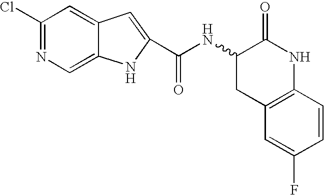

The title compound was prepared from 5-chloro-1H-pyrrolo[2,3-c]pyridine-2-carboxylic acid (Preparation 3) and the appropriate amine as described in Example 12. m/z (ES⁺)=359.01 [M+H]⁺; RT=3.31 min.

### Example 14

### {3-[(5-Chloro-1H-pyrrolo[2,3-c]pyridine-2-carbonyl)amino]-7-fluoro-2-oxo-3,4-dihydro-2H-quinolin-1-yl}acetic acid

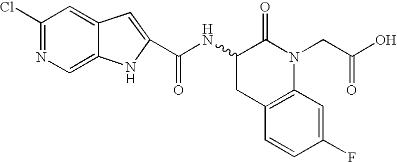

{3-[(5-Chloro-1H-pyrrolo[2,3-c]pyridine-2-carbonyl)amino]-7-fluoro-2-oxo-3,4-dihydro-2H-quinolin-1-yl}acetic acid methyl ester (Example 12) was hydrolysed according to the method of Example 7 to afford the title compound. δ_{H} (d₆ DMSO): 8.59 (1H, s), 7.69 (1H, s), 7.33-7.26 (1H, m), 7.18 (1H, s), 6.87-6.79 (2H, m), 4.98-4.76 (2H, m), 4.58 (1H, d), 3.24-3.18 (2H, m); m/z (ES⁺)=417.02 [M+H]⁺; RT=3.14 min.

### Example 15

### 5-Chloro-1H-pyrrolo[2,3-c]pyridine-2-carboxylic acid [7-fluoro-2-hydroxyethyl)-2-oxo-1,2,3,4-tetrahydroquinolin-3-yl}amide

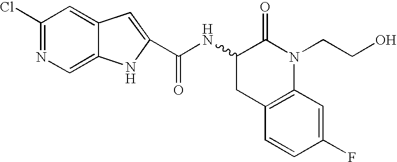

5-Chloro-1H-pyrrolo[2,3-c]pyridine-2-carboxylic acid {1-[2-(tert-butyldimethylsilyloxy)ethyl]-7-fluoro-2-oxo-1,2,3,4-tetrahydroquinolin-3-yl}amide (Preparation 42) was deprotected according to Example 9 to afford the title compound. δ_{H} (CD₃OD): 8.59 (1H, s), 7.68 (1H, s), 7.30-7.23 (1H, m), 7.21-7.13 (2H, m), 6.85-6.77 (1H, m), 4.90-4.80 (1H, m), 4.19-4.00 (2H, m), 3.86-3.72 (2H, m), 3.22-3.11 (2H, m); m/z (ES⁺)=403.03 [M+H]⁺; RT=3.21 min.

### Example 16

### 5-Chloro-1H-pyrrolo[2,3-c]pyridine-2-carboxylic acid [1-(3-hydroxypropyl)-2-oxo-1,2,3,4-tetrahydrquinolin-3-yl]amide

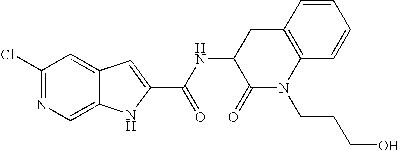

To a stirred solution of 5-chloro-1H-pyrrolo[2,3-c]pyridine-2-carboxylic acid {1-[3-(tert-butyldimethylsilanyloxy)propyl]-2-oxo-1,2,3,4-tetrahydroquinolin-3-yl}amide (Preparation 43, 316 mg, 0.62 mmol) in THF (10 mL) was added TBAF (1.0M in THF, 0.92 mL, 0.92 mmol). The reaction was stir at rt for 16 h. The solvent was removed in vacuo and the residue dissolved in methanol (20 mL). The precipitate which formed was filtered, washed with methanol, and dried under vacuum affording the title compound as a white crystalline solid. δ_{H} (d₆ DMSO): 1.63-1.80 (2H, m), 3.03-3.19 (2H, m), 3.44-3.50 (2H, m), 3.95-4.00 (2H, m), 4.56 (1H, t), 4.70-4.78 (1H, m), 7.05 (1H, t), 7.22-7.32 (4H, m), 7.78 (1H, s), 8.59 (1H, s), 9.10 (1H, d); m/z (ES⁺) 399 [M+H]⁺; RT=3.11 min.

### Example 17

### 5-Chloro-1H-pyrrolo[2,3-c]pyridine-2-carboxylic acid [1 (N-hydroxycarbamimidoylmethyl)-2-oxo-1,2,3,4-tetrahydroquinolin-3-yl]amide

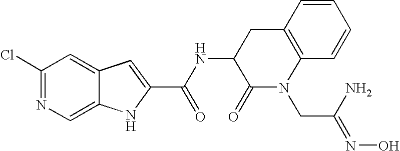

Hydroxylamine hydrochloride (188 mg, 2.71 mmol) was added to a stirred solution of sodium methoxide (146 mg, 2.71 mmol) in methanol (15 mL) under an argon atmosphere. To this was added 5-chloro-1H-pyrrolo[2,3-c]pyridine-2-carboxylic acid (1-cyanomethyl-2-oxo-1,2,3,4-tetrahydroquinolin-3-yl)amide (Example 54, 514 mg, 1.35 mmol) and THF (10 mL) and the reaction mixture stirred at rt for 72 h. To this was added a solution of sodium methoxide (73 mg, 1.35 mmol) and hydroxylamine hydrochloride (94 mg, 1.35 mmol) in methanol (5 mL) and the reaction mixture stirred at rt for 24 h. The solvent was removed in vacuo and the residue triturated with methanol affording the title compound as a white solid. δ_{H} (d₆ DMSO): 3.07 (1H, dd), 3.21 (1H, t), 4.29 (1H, d), 4.76 (1H, d), 4.79-4.86 (1H, m), 5.41 (1H, br s), 7.05 (1H, t), 7.16 (1H, d), 7.25-7.30 (3H, m), 7.79 (1H, s), 8.59 (1H, s), 9.13 (1H, d), 9.18 (1H, s), 12.36 (1H, s); m/z (ES⁺) 413 [M+H]⁺; RT=2.67 min.

### Example 18

### 5-Chloro-1H-pyrrolo[2,3-c]pyridine-2-carboxylic acid [1-(2-methanesulfinylethyl)-2-oxo-1,2,3,4-tetrahydroquinolin-3-yl]amide

### Example 19

### 5-Chloro-1H-pyrrolo[2,3-c]pyridine-2-carboxylic acid [1-(2-methanesulfonylethyl)-2-oxo-1,2,3,4-tetrahydroquinolin-3-yl]amide

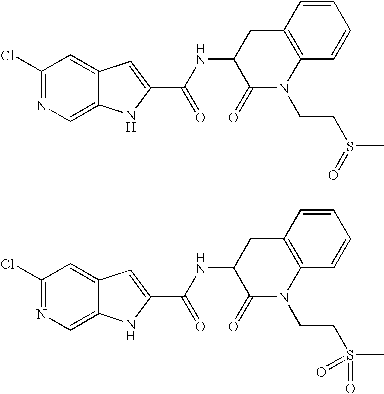

Oxone (370 mg, 0.60 mmol) in H₂O (10 mL) was added to a suspension of 5-chloro-1H-pyrrolo[2,3-c]pyridine-2-carboxylic acid [1-(2-methylsulfanylethyl)-2-oxo-1,2,3,4-tetrahydroquinolin-3-yl]amide (Example 58, 250 mg, 0.60 mmol), in methanol (10 mL). The reaction mixture was stirred at rt for 16 h. The reaction mixture was diluted with EtOAc (100 mL) and washed with saturated NaHCO₃ (100 mL). The aqueous phase was extracted into DCM:methanol (9:1, 4×150 mL) and the combined organics washed with brine (2×50 mL), dried (MgSO₄) and concentrated in vacuo. The residue was purified by chromatography on silica gel eluting with DCM:methanol (97:3 to 95:5) affording the desired products as white solids.

### 5-Chloro-1H-pyrrolo[2,3-c]pyridine-2-carboxylic acid [2-methanesulfinylethyl)-2-oxo-1,2,3,4-tetrahydroquinolin-3-yl]amide

δ_{H} (d₆ DMSO): 2.60, 2.61 (3H, 2×s), 2.92-3.00 (1H, m), 3.04-3.22 (3H, m), 4.28-4.34 (2H, m), 4.73-4.81 (1H, m), 7.08 (1H, t), 7.27-7.35 (4H, m), 7.79 (1H, s), 8.59 (1H, s), 9.11-9.15 (1H, m), 12.35 (1H, s); m/z (ES⁺) 431 [M+H]⁺; RT=3.01 min.

### 5-Chloro-1H-pyrrolo[2,3-c]pyridine-2-carboxylic acid [1-(2-methanesulfonylethyl)-2-oxo-1,2,3,4-tetrahydroquinolin-3-yl]amide

δ_{H} (d₆ DMSO): 3.06 (1H, dd), 3.08 (3H, s), 3.20 (1H, app. t), 3.41-3.49 (2H, m), 4.35 (2H, t), 4.74-4.81 (1H, m), 7.09 (1H, t), 7.23 (1H, d), 7.27 (1H, s), 7.31-7.37 (2H, m), 7.79 (1H, s), 8.59 (1H, s), 9.13 (1H, d), 12.35 (1H, s); m/z (ES⁺) 447 [M+H]⁺; RT=3.14 min.

### Example 20

### 5-Chloro-1H-pyrrolo[2,3-c]pyridine-2-carboxylic acid [2-oxo-1-(1H-tetrazol-5-ylmethyl)-1,2,3,4-tetrahydroquinolin-3-yl]amide

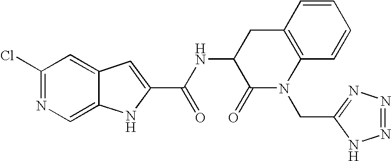

To a solution of 5-chloro-1H-pyrrolo[2,3-c]pyridine-2-carboxylic acid (1-cyanomethyl-2-oxo-1,2,3,4-tetrahydroquinolin-3-yl)amide (Example 54, 200 mg, 0.53 mmol) in NMP (7 mL) was added sodium azide (120 mg, 1.84 mmol) and triethylamine hydrochloride (239 mg, 1.74 mmol). The reaction mixture was heated to 150° C. for 3 h, then cooled to rt and partitioned between citric acid solution (2M, 40 mL) and EtOAc (3×40 mL). The combined organic fractions were washed with brine (2×30 mL), dried (MgSO₄) and concentrated in vacuo. The residue was diluted with methanol (20 mL) affording a brown precipitate which was filtered, washed with methanol, and dried under vacuum affording the title product as a brown solid. δ_{H} (d₆ DMSO): 3.12 (1H, dd), 3.29 (1H, t), 4.91-4.99 (1H, m), 5.31 (1H, d), 5.59 (1H, d), 7.07-7.10 (2H, m), 7.26-7.29 (2H, m), 7.34 (1H, d), 7.79 (1H, s), 8.59 (1H, s), 9.19 (1H, d), 12.35 (1H, s); m/z (ES⁺) 423 [M+H]⁺; RT=3.11 min.

### Example 21

### 5-Chloro-1H-pyrrolo[2,3-c]pyridine-2-carboxylic acid [1-(2-hydroxyethyl)-2-oxo-1,2,3,4-tetrahydroquinolin-3-yl]amide

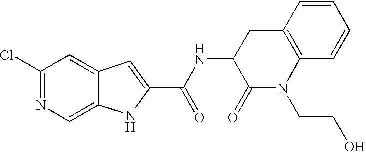

To a stirred solution of 5-chloro-1H-pyrrolo[2,3-c]pyridine-2-carboxylic acid {1-[2-(tert-butyldimethylsilanyloxy)ethyl]-2-oxo-1,2,3,4-tetrahydroquinolin-3-yl}amide (Preparation 44, 200 mg, 0.40 mmol) in THF (5 mL) was added TBAF (1.0M in THF, 0.60 mL, 0.60 mmol). The reaction was stirred at rt for 16 h. The solvent was removed in vacuo and the residue partitioned between water (30 mL) and EtOAc (2×30 mL). The combined organic fractions were dried (MgSO₄) concentrated in vacuo and purified by chromatography on silica gel eluting with methanol:DCM (3:47) affording the title compound as a white solid. δ_{H} (d₆ DMSO) 3.05 (1H, dd), 3.16 (1H, t), 3.55-3.64 (2H, m), 3.88-3.95 (1H, m), 4.01-4.08 (1H, m), 4.73-4.81 (1H, m), 4.86 (1H, t), 7.03-7.08 (1H, m), 7.27-7.31 (4H, m), 7.79 (1H, s), 8.59 (1H, s), 9.11 (1H, d), 12.35 (1H, s); m/z (ES⁺) 385 [M+H]⁺; RT=3.15 min.

### Example 22

### {3-[(5-Chloro-1H-pyrrolo[2,3-c]pyridine-2-carbonyl)amino]-2-oxo-3,4-dihydro-2H-quinolin-1-yl}acetic acid methyl ester

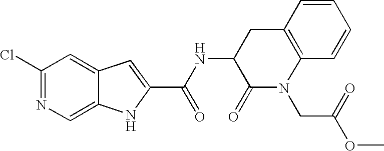

DMTMM (465 mg, 1.68 mmol) was added to a suspension of 5-chloro-1H-pyrrolo[2,3-c]pyridine-2-carboxylic acid (Preparation 3, 300 mg, 1.53 mmol) and (3-amino-2-oxo-3,4-dihydro-2H-quinolin-1-yl)acetic acid methyl ester (WO03/074532, 393 mg, 1.68 mmol) in ethanol (15 mL), and the reaction mixture was stirred at rt for 16 h. The solvent was removed in vacuo and the residue partitioned between water (50 mL) and EtOAc (4×50 mL). The combined organic fractions were concentrated in vacuo and the residue triturated with ether affording the title compound as a beige solid. m/z (ES⁺) 413 [M+H]⁺; RT=3.32 min.

### Example 23

### {3-[(5-Chloro-1H-pyrrolo[2,3-c]pyridine-2-carbonyl)amino]-2-oxo-3,4-dihydro-2H-quinolin-1-yl}acetic acid

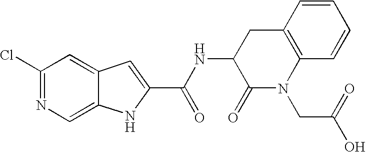

To a stirred suspension of {3-[(5-chloro-1H-pyrrolo[2,3-c]pyridine-2-carbonyl)amino]-2-oxo-3,4-dihydro-2H-quinolin-1-yl}acetic acid methyl ester (Example 22, 2 g, 4.84 mmol) in THF (100 mL) was added LiOH solution (2M, 4.8 mL, 9.69 mmol). The reaction mixture was stirred at rt for 16 h. The solvent was removed in vacuo and the residue dissolved in water (100 mL) and acidified with hydrochloric acid solution (2M) affording a beige precipitate, which was filtered and dried under air affording the title compound as a beige solid. δ_{H} (d₆ DMSO): 3.09 (1H, dd), 3.23 (1H, t), 4.54 (1H, d), 4.74 (1H, d), 4.77-4.84 (1H, m), 7.02 (1H, d), 7.07 (1H, t), 7.27 (1H, s), 7.29-7.32 (2H, m), 7.79 (1H, s), 8.59 (1H, s), 9.19 (1H, d), 12.36 (1H, s); m/z (ES⁺) 399 [M+H]⁺; RT=3.07 min.

### Example 24

### (2-{3-[(5-Chloro-1H-pyrrolo[2,3-c]pyridine-2-carbonyl)amino]-2-oxo-3,4-dihydro-2H-quinolin-1-yl}ethyl)carbamic acid tert-butyl ester

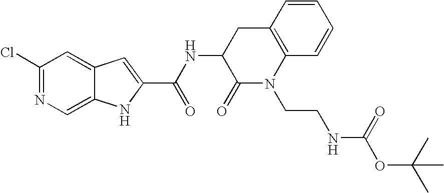

Sodium borohydride (1 g, 26.3 mmol) was added to a suspension of 5-chloro-1H-pyrrolo[2,3-c]pyridine-2-carboxylic acid (1-cyanomethyl-2-oxo-1,2,3,4-tetrahydroquinolin-3-yl)amide (Example 54, 1 g, 2.63 mmol), cobalt chloride hexahydrate (1.25 g, 5.27 mmol) and tert-butyl dicarbonate (1.15 g, 5.27 mmol) in THF (10 mL) and methanol (15 mL) over a period of 15 min. The reaction mixture was stirred at rt for 5 days. The reaction mixture was filtered through celite and washed with methanol:H₂O (9:1, 100 mL). The filtrate was concentrated in vacuo and the residue partitioned between saturated NaHCO₃ solution (100 mL) and EtOAc (3×50 mL). The combined organic fractions were dried (MgSO₄) concentrated in vacuo and purified by chromatography on silica gel eluting with methanol:DCM (1:24) affording the title compound as a beige solid. δ_{H} (d₆ DMSO): 1.33 (9H, s), 3.05 (1H, dd), 3.15-3.20 (3H, m), 3.79-3.86 (1H, m), 3.97-4.04 (1H, m), 4.74-4.81 (1H, m), 7.00-7.07 (2H, m), 7.27-7.32 (4H, m), 7.78 (1H, s), 8.59 (1H, s), 9.08 (1H, d), 12.34 (1H, s); m/z (ES⁺) 484 [M+H]⁺; RT=3.82 min.

### Example 25

### 5-Chloro-1H-pyrrolo[2,3-c]pyridine-2-carboxylic acid [1-(2-aminoethyl)-2-oxo-1,2,3,4-tetrahydroquinolin-3-yl]amide trifluoroacetate

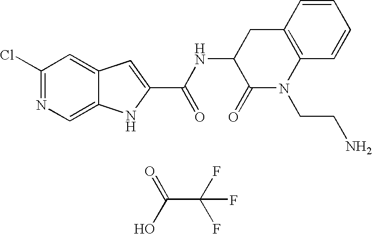

(2-{3-[(5-Chloro-1H-pyrrolo[2,3-c]pyridine-2-carbonyl)amino]-2-oxo-3,4-dihydro-2H-quinolin-1-yl}ethyl)carbamic acid tert-butyl ester (Example 24, 620 mg, 1.28 mmol) was dissolved in TFA:H₂O (9:1, 5 mL) and the reaction stirred at rt for 2 h. Toluene (20 mL) was added to the reaction mixture and the solvent concentrated in vacuo affording the title compound as a yellow solid. δ_{H} (d₆ DMSO): 3.05-3.10 (3H, m), 3.22 (1H, t), 3.97-4.04 (1H, m), 4.26-4.33 (1H, m), 4.83-4.90 (1H, m), 7.09 (1H, t), 7.23-7.27 (2H, m), 7.31-7.35 (2H, m), 7.78 (1H, s), 7.94 (3H, br s), 8.60 1H, s), 9.12 (1H, d), 12.36 (1H, s); m/z (ES⁺) 384 [M+H]⁺; RT=2.65 min.

### Example 26

### 5-Chloro-1H-pyrrolo[2,3-c]pyridine-2-carboxylic acid [1-(2-methanesulfonylaminoethyl)-2-oxo-1,2,3,4-tetrahydroquinolin-3-yl]amide

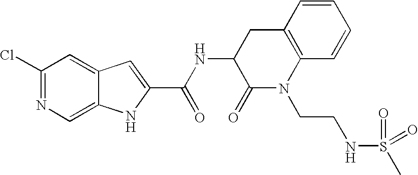

Methane sulfonyl chloride (34 μL, 0.44 mmol) was added to a solution of 5-chloro-1H-pyrrolo[2,3-c]pyridine-2-carboxylic acid [1-(2-aminoethyl)-2-oxo-1,2,3,4-tetrahydroquinolin-3-yl]amide trifluoroacetate (Example 25, 200 mg, 0.40 mmol) in DCM (10 mL) and triethylamine (188 μL, 0.84 mmol) at 0° C. The reaction mixture was stirred at 0° C. for 1 h, then rt for 16 h. The solvent was removed in vacuo and the residue triturated with methanol (20 mL) affording the title compound as an off-white solid. m/z (ES⁺) 462 [M+H]⁺; RT=3.26 min.

### Example 27

### 5-Chloro-1H-pyrrolo[2,3-c]pyridine-2-carboxylic acid [1-(2-acetylaminoethyl)-2-oxo-1,2,3,4-tetrahydroquinolin-3-yl]amide

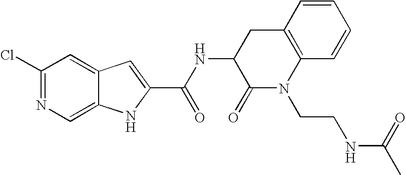

5-Chloro-1H-pyrrolo[2,3-c]pyridine-2-carboxylic acid [1-(2-amino-ethyl)-2-oxo-1,2,3,4-tetrahydroquinolin-3-yl]amide trifluoroacetate (Example 25, 200 mg, 0.40 mmol) was added to a solution of HOBt (54 mg, 0.40 mmol) in DMF (5 mL), acetic acid (23 μL, 0.40 mmol) and DIPEA (0.22 mL, 1.25 mmol). After 5 min, EDCI (100 mg, 0.52 mmol) was added and the reaction mixture stirred at rt for 16 h. The solvent was removed in vacuo and the residue partitioned between water (30 mL) and EtOAc (3×30 mL). The combined organic fractions were dried (MgSO₄), concentrated in vacuo and purified by chromatography on silica gel eluting with methanol:DCM (1:19) affording the title compound as a white solid. δ_{H} (d₆ DMSO): 1.76 (3H, s), 3.05 (1H, dd), 3.14-3.17 (1H, m), 3.24-3.29 (2H, m), 3.81-3.88 (1H, m), 3.97-4.04 (1H, m), 4.74-4.81 (1H, m), 7.03-7.07 (1H, m), 7.27 (1H, s), 7.30-7.35 (3H, m), 7.78 (1H, s), 8.07 (1H, t), 8.59 (1H, s), 9.09 (1H, d), 12.34 (1H, s); m/z (ES⁺) 426 [M+H]⁺; RT=3.15 min.

### Example 28

### 5-Chloro-1H-pyrrolo[2,3-c]pyridine-2-carboxylic acid {1-[2-(4-hydroxypiperidin-1-yl)-2-oxoethyl]-2-oxo-1,2,3,4-tetrahydroquinolin-3-yl}amide

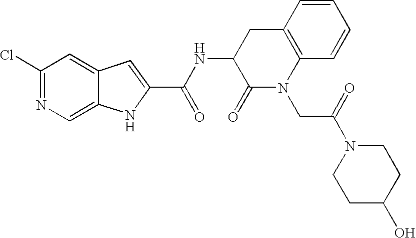

DMTMM (83 mg, 0.30 mmol) was added to a stirred solution of {3-[(5-chloro-1H-pyrrolo[2,3-c]pyridine-2-carbonyl)amino]-2-oxo-3,4-dihydro-2H-quinolin-1-yl}acetic acid (Example 22, 100 mg, 0.25 mmol) and 4-hydroxypiperidine hydrochloride (38 mg, 0.28 mmol) in ethanol (10 mL) and 4-methylmorpholine (30 μL, 0.28 mmol). The reaction was stirred at rt for 16 h. The solvent was removed in vacuo and the residue triturated with H₂O affording the title compound as a white solid. m/z (ES⁺) 482 [M+H]⁺; RT=2.97 min.

### Examples 29-47

The procedure described in Example 28 was used to prepare the compounds of Examples 29-47 from {3-[(5-chloro-1H-pyrrolo[2,3-c]pyridine-2-carbonyl)amino]-2-oxo-3,4-dihydro-2H-quinolin-1-yl}acetic acid (Example 22) and the appropriate amine.

TABLE

| | | | RT | |
|---|---|---|---|---|
| Ex | Structure | Name | min) | m/z (ES⁺) |
| 29 | 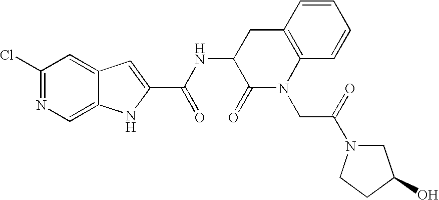 | 5-Chloro-1H-pyrrolo[2,3-c]pyridine-2-carboxylic acid{1-[2-(3-hydroxypyrrolidin-1-yl)-2-oxo-ethyl]-2-oxo-1,2,3,4-tetrahydroquinolin-3-yl}amide | 2.99 | 468[M + H]⁺ |
| 30 | 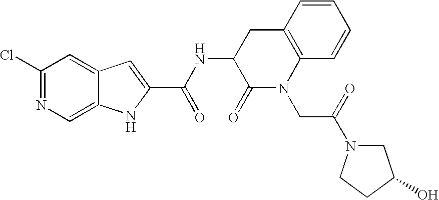 | 5-Chloro-1H-pyrrolo[2,3-c]pyridine-2-carboxylic acid{1-[2-(3-hydroxypyrrolidin-1-yl)-2-oxo-ethyl]-2-oxo-1,2,3,4-tetrahydroquinolin-3-yl}amide | 3.01 | 468[M + H]⁺ |
| 31 | 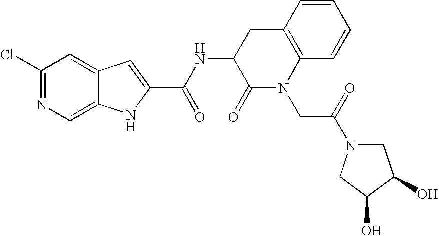 | 5-Chloro-1H-pyrrolo[2,3-c]pyridine-2-carboxylic acid{1-[2-(3,4-dihydroxy-pyrrolidin-1-yl)-2-oxo-ethyl]-2-oxo-1,2,3,4-tetrahydro-quinolin-3-yl}amide | 2.89 | 484[M + H]⁺ |
| 32 | 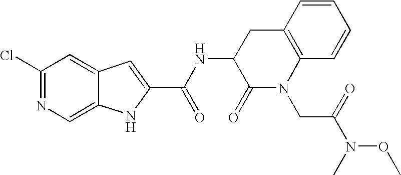 | 5-Chloro-1H-pyrrolo[2,3-c]pyridine-2-carboxylic acid{1-[(methoxymethyl-carbamoyl)methyl]-2-oxo-1,2,3,4-tetrahydroquinolin-3-yl}amide | 3.44 | 442[M + H]⁺ |
| 33 | 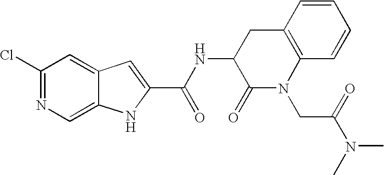 | 5-Chloro-1H-pyrrolo[2,3-c]pyridine-2-carboxylic acid (1-dimethylcarbamoylmethyl-2-oxo-1,2,3,4-tetrahydro-quinolin-3-yl)amide | 2.90 | 426[M + H]⁺ |
| 34 | 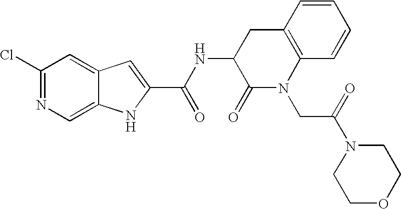 | 5-Chloro-1H-pyrrolo[2,3-c]pyridine-2-carboxylic acid [1-(2-morpholin-4-yl-2-oxo-ethyl)-2-oxo-1,2,3,4-tetrahydro-quinolin-3-yl]amide | 3.04 | 468[M + H]⁺ |
| 35 | 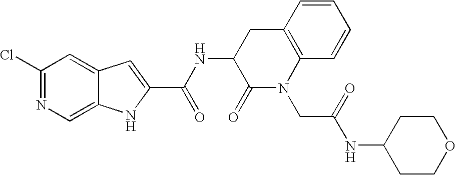 | 5-Chloro-1H-pyrrolo[2,3-c]pyridine-2-carboxylic acid{2-oxo-1-[(tetrahydropyran-4-ylcarbamoyl)methyl]-1,2,3,4-tetrahydroquinolin-3-yl}amide | 2.90 | 482[M + H]⁺ |
| 36 | 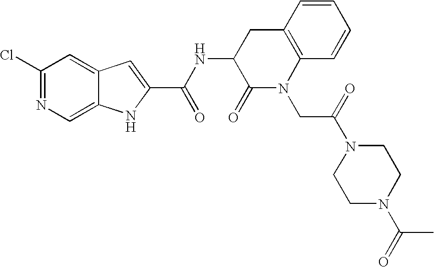 | 5-Chloro-1H-pyrrolo[2,3-c]pyridine-2-carboxylic acid{1-[2-(4-acetylpiperazin-1-yl)-2-oxo-ethyl]-2-oxo-1,2,3,4-tetrahydroquinolin-3-yl}amide | 2.99 | 509[M + H]⁺ |
| 37 | 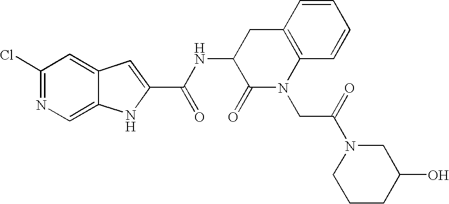 | 5-Chloro-1H-pyrrolo[2,3-c]pyridine-2-carboxylic acid{1-[2-(3-hydroxypiperidin-1-yl)-2-oxo-ethyl]-2-oxo-1,2,3,4-tetrahydroquinolin-3-yl}amide | 3.07 | 482[M + H]⁺ |
| 38 | 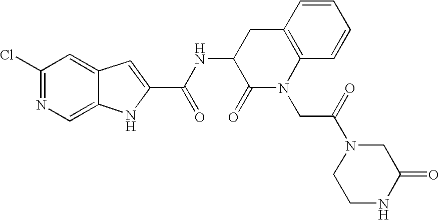 | 5-Chloro-1H-pyrrolo[2,3-c]pyridine-2-carboxylic acid{2-oxo-1-[2-oxo-2-(3-oxo-piperazin-1-yl)ethyl]-1,2,3,4-tetrahydroquinolin-3-yl}amide | 3.06 | 481[M + H]⁺ |
| 39 | 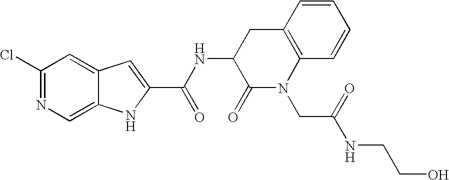 | 5-Chloro-1H-pyrrolo[2,3-c]pyridine-2-carboxylic acid{1-[(2-hydroxyethyl-carbamoyl)methyl]-2-oxo-1,2,3,4-tetrahydroquinolin-3-yl}amide | 2.90 | 442[M + H]⁺ |
| 40 | 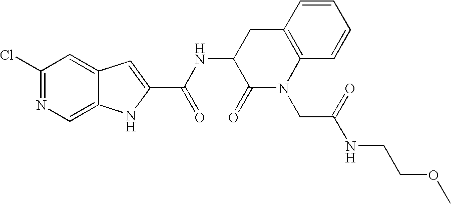 | 5-Chloro-1H-pyrrolo[2,3-c]pyridine-2-carboxylic acid{1-[(2-methoxyethyl-carbamoyl)methyl]-2-oxo-1,2,3,4-tetrahydroquinolin-3-yl}amide | 3.26 | 456[M + H]⁺ |
| 41 | 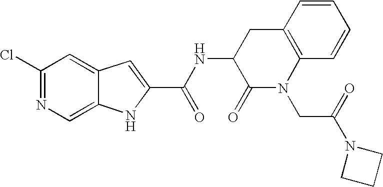 | 5-Chloro-1H-pyrrolo[2,3-c]pyridine-2-carboxylic acid [1-(2-azetidin-1-yl-2-oxoethyl)-2-oxo-1,2,3,4-tetrahydroquinolin-3-yl]amide | 3.09 | 438[M + H]⁺ |
| 42 | 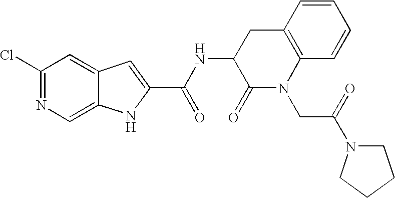 | 5-Chloro-1H-pyrrolo[2,3-c]pyridine-2-carboxylic acid [2-oxo-1-(2-oxo-2-pyrrolidin-1-yl-ethyl)-1,2,3,4-tetrahydro-quinolin-3-yl]amide | 3.15 | 452[M + H]⁺ |
| 43 | 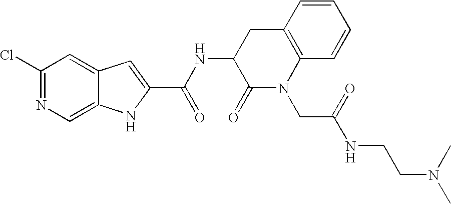 | 5-Chloro-1H-pyrrolo[2,3-c]pyridine-2-carboxylic acid{1-[(2-dimethylamino-ethylcarbamoyl)methyl]-2-oxo-1,2,3,4-telrahydroquinolin-3-yl}amide | 2.77 | 469[M + H]⁺ |
| 44 | 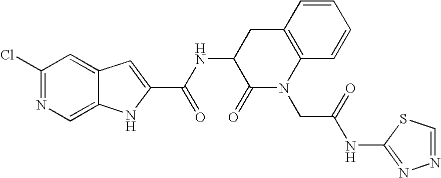 | 5-Chloro-1H-pyrrolo[2,3-c]pyridine-2-carboxylic acid [2-oxo-1-([1,3,4]thiadiazol-2-ylcarbamoylmethyl)-1,2,3,4-tetrahydroquinolin-3-yl]amide | 3.12 | 482[M + H]⁺ |
| 45 | 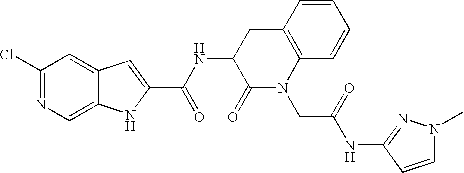 | 5-Chloro-1H-pyrrolo[2,3-c]pyridine-2-carboxylic acid{1-[(1-methyl-1H-pyrazol-3-ylcarbamoyl)methyl]-2-oxo-1,2,3,4-tetrahydroquinolin-3-yl}amide | 3.07 | 478[M + H]⁺ |
| 46 | 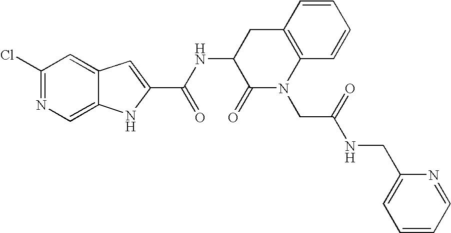 | 5-Chloro-1H-pyrrolo[2,3-c]pyridine-2-carboxylic acid (2-oxo-1-{[(pyridin-2-ylmethyl)-carbamoyl]methyl}-1,2,3,4-tetrahydroquinolin-3-yl)amide | 2.90 | 489[M + H]⁺ |
| 47 | 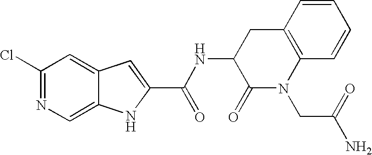 | 5-Chloro-1H-pyrrolo[2,3-c]pyridine-2-carboxylic acid (1-carbamoylmethyl-2-oxo-,2,3,4-tetrahydroquinolin-3-yl)amide | 2.92 | 398[M + H]⁺ |

### Example 48

### 5-Chloro-1H-pyrrolo[2,3-c]pyridine-2-carboxylic acid (2-oxo-1,2,3,4-tetrahydroquinolin-3-yl)amide

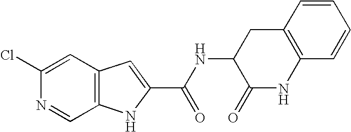

5-Chloro-1H-pyrrolo[2,3-c]pyridine-2-carboxylic acid (Preparation 3, 50 mg, 0.25 mmol), 3-amino-3,4-dihydro-1H-quinolin-2-one (Preparation 15, 41 mg, 0.25 mmol) and HOBt (34 mg, 0.25 mmol) were dissolved in DMF (5 mL) and DIPEA (89 μL, 0.51 mmol). After 5 min, EDCI (63 mg, 0.33 mmol) was added and the reaction mixture stirred at rt for 16 h. The solvent was removed in vacuo and the residue partitioned between water (30 mL) and EtOAc (3×30 mL). The combined organic fractions were washed with brine (20 mL), dried (MgSO₄) and concentrated in vacuo. Purification by chromatography on silica gel eluting with methanol:DCM (1:24) afforded the title compound as a yellow solid. δ_{H} (CD₃OD): 3.26 (2H, d), 4.93 (1H, t), 6.95 (1H, d), 7.06 (1H, t), 7.24 (1H, s), 7.26-7.31 (2H, m), 7.74 (1H, s), 8.65 (1H, s); m/z (ES⁺) 341 [M+H]⁺; RT=3.04 min.

### Examples 49-59

The procedure described in Example 48 was used to prepare the compounds of Examples 49-59 from 5-chloro-1H-pyrrolo[2,3-c]pyridine-2-carboxylic acid (Preparation 3) and the appropriate amine.

TABLE

| | | | RT | |
|---|---|---|---|---|
| Ex | Structure | Amine | (min) | m/z (ES⁺) |
| 49 | 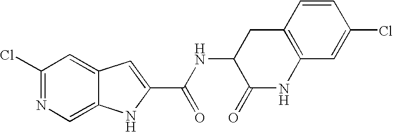 | Preparation 19 | 3.22 | 375[M + H]⁺ |
| 50 | 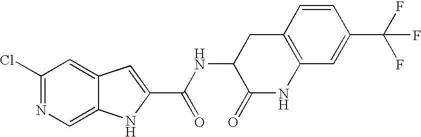 | Preparation 20 | 3.45 | 409[M + H]⁺ |
| 51 | 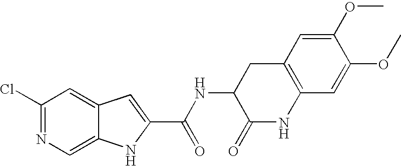 | Preparation 21 | 3.01 | 401[M + H]⁺ |
| 52 | 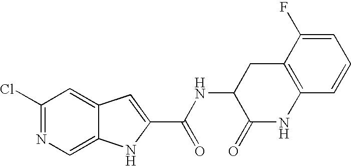 | Preparation 22 | 3.14 | 359[M + H]⁺ |
| 53 | 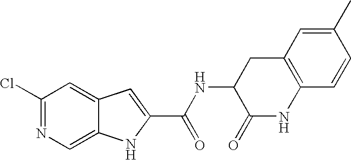 | Preparation 23 | 3.20 | 355[M + H]⁺ |
| 54 | 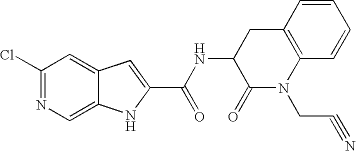 | WO03/074532 | 3.42 | 380[M + H]⁺ |
| 55 | 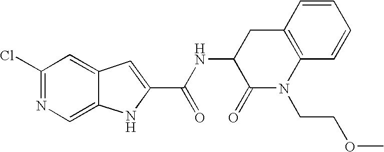 | WO03/074532 | 3.42 | 399[M + H]⁺ |
| 56 | 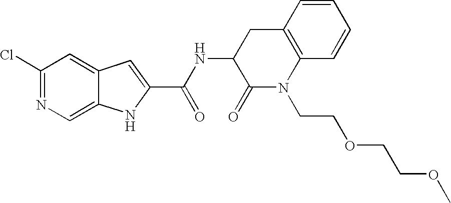 | Preparation 18 | 3.42 | 443[M + H]⁺ |
| 57 | 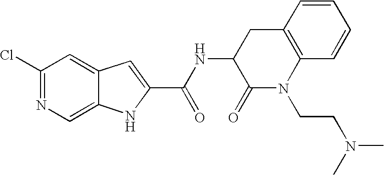 | WO03/074532 | 2.72 | 412[M + H]⁺ |
| 58 | 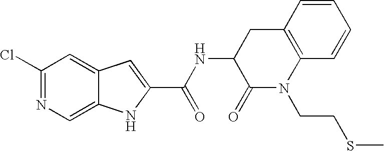 | WO03/074532 | 3.52 | 415[M + H]⁺ |
| 59 | 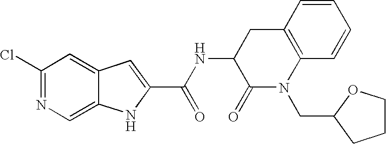 | Preparation 17 | 3.37 | 425[M + H]⁺ |

### Examples 60-63

The procedure described in Example 48 was used to prepare the compounds of Examples 60-63 from 5-chloro-1H-pyrrolo[2,3-b]pyridine-2-carboxylic acid (Preparation 5) and the appropriate amine.

TABLE

| | | | RT | |
|---|---|---|---|---|
| Ex | Structure | Amine | (min) | m/z (ES⁺) |
| 60 | 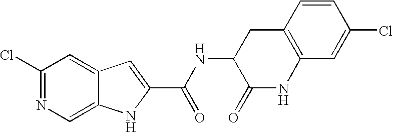 | Preparation 19 | 3.61 | 3.75[M + H]⁺ |
| 61 | 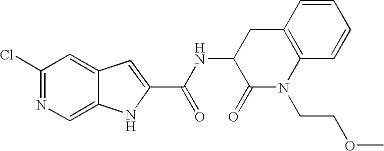 | WO03/074532 | 3.54 | 399[M + H]⁺ |
| 62 | 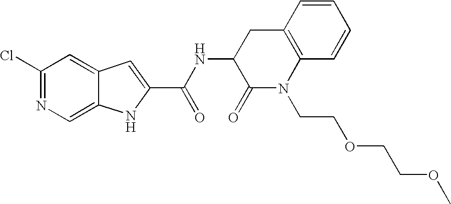 | Preparation 18 | 3.51 | 443[M + H]⁺ |
| 63 | 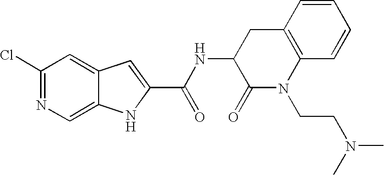 | WO03/074532 | 2.69 | 412[M + H]⁺ |

### Example 64

### {7-chloro-3-[(5-chloro-1H-pyrrolo[2,3-c]pyridine-2-carbonyl)amino]-2-oxo-3,4-dihydro-2H-quinolin-1-yl}acetic acid methyl ester

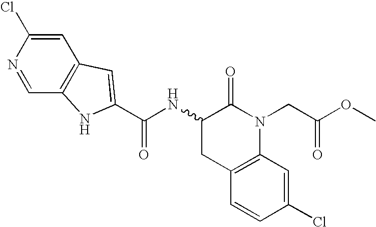

To a solution of 5-chloro-1H-pyrrolo[2,3-c]pyridine-2-carboxylic acid (Preparation 3, 240 mg, 1.22 mmol), EDCI (292 mg, 1.51 mmol) and HOBt monohydrate (190 mg, 1.24 mmol) in DMF (15 mL) was added 3-amino-7-chloro-2-oxo-3,4-dihydro-2H-quinolin-1-yl)acetic acid methyl ester (Preparation 50, 330 mg, 1.23 mmol) and DIPEA (0.47 mL, 2.70 mmol). The resulting solution was stirred for 12 h at rt before the reaction mixture was partitioned between EtOAc (50 mL) and water/brine (100 mL, 1:1). The layers were separated and the aqueous phase extracted with EtOAc (3×50 mL), then the combined organics were washed with dilute HCl solution (1M, 50 mL), dilute NaOH solution (1M, 50 mL) and brine (50 mL). The organic phase was dried (MgSO₄), filtered and concentrated in vacuo. Addition of methanol (˜2 mL) led to the precipitation of the title compound, which was obtained as an off-white solid after filtration. δ_{H} (d₆ DMSO): 3.10-3.24 (2H, m), 3.70 (3H, s), 4.72 (1H, d), 4.78-4.86 (2H, m), 7.15 (1H, dd), 7.24 (1H, d), 7.26 (1H, s), 7.36 (1H, d), 7.80 (1H, s), 8.60 (1H, s), 9.29 (1H, d), 12.36 (1H, br s); m/z (ES⁺)=446.99 [M+H]⁺; RT=3.45 min.

### Example 65

### {7-Chloro-3-[(5-chloro-1H-pyrrolo[2,3-c]pyridine-2-carbonyl)amino]-2-oxo-3,4-dihydro-2H-quinolin-1-yl}acetic acid

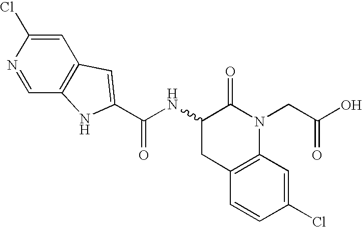

To a solution of {7-chloro-3-[(5-chloro-1H-pyrrolo[2,3-c]pyridine-2-carbonyl)amino]-2-oxo-3,4-dihydro-2H-quinolin-1-yl}acetic acid methyl ester (Example 64, 100 mg, 0.22 mmol) in a mixture of methanol and DMSO (1/1, 10 mL) was added NaOH solution (1N, 0.5 mL). After stirring for 2 h at 80° C. the solvents were removed in vacuo and the resulting oil taken up in water (100 mL). The aqueous layer was washed with EtOAc (30 mL) and then made acidic with dilute hydrochloric acid (1N). Extraction with EtOAc (4×50 mL), washing of the combined extracts with brine (50 mL) and concentration after drying (MgSO₄) gave a crude product. Purification by preparative thin layer chromatography on silica gel (eluent: DCM/methanol/acetic acid/water: 160/30/5/3) gave the title compound as colourless wax. δ_{H} (d₄ MeOH): 3.21-3.24 (2H, m), 4.61, 485 (2H, 2×d), 4.94 (1H, dd), 7.04 (1H, d), 7.10 (1H, dd), 7.19 (1H, s), 7.28 (1H, d), 7.69 (1H, s), 8.59 (1H, s); m/z (ES⁻)=431.03 [M−H]⁻; RT=3.26 min.

### Example 66

### 5-Chloro-1H-pyrrolo[2,3-c]pyridine-2-carboxylic acid [7-chloro-1-(2-hydroxyethyl)-2-oxo-1,2,3,4-tetrahydroquinolin-3-yl]amide

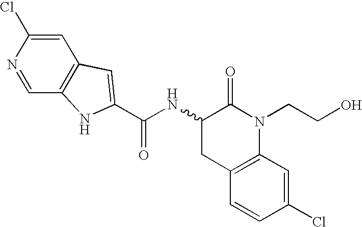

The title compound was obtained as a side product in the preparation of Preparation 64. TLC (hexane/ethyl acetate: 1/3): R_{f} 0.15; δ_{H} (d₆ DMSO): 3.07-3.18 (2H, m), 3.61 (2H, m), 3.89 (1H, m), 4.04 (1H, m), 4.81 (1H, m), 4.92 (1H, appt), 7.12 (1H, dd), 7.27 (1H, s), 7.31 (1H, d), 7.43 (1H, d), 7.80 (1H, s), 8.60 (1H, s), 9.13 (1H, d), 12.36 (1H, br s); m/z (ES⁺)=419.11 [M+H]⁺; RT=3.42 min.

### Example 67

### 6-Chloro-1H-pyrrolo[3,2-c]pyridine-2-carboxylic acid (2-oxo-1,2,3,4-tetrahydroquinolin-3-yl)amide

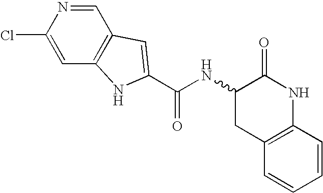

To a solution of 6-chloro-1H-pyrrolo[3,2-c]pyridine-2-carboxylic acid (Preparation 14, 40 mg, 0.20 mmol), EDCI (39.5 mg, 0.21 mmol) and HOBt monohydrate (31 mg, 0.20 mmol) in DMF (3 mL) was added 3-amino-3,4-dihydro-1H-quinolin-2-one hydrochloride (A. L. Davies et al., Arch. Biochem. Biophys., 102, 1963, 48-51, 43.8 mg, 0.20 mmol) and DIPEA (114 μL, 0.65 mmol). The resulting solution was stirred for 12 h at rt before the reaction mixture was partitioned between EtOAc (50 mL) and water/brine (100 mL, 1:1). The layers were separated and the aqueous phase extracted with EtOAc (3×50 mL), then the combined organics were washed with dilute HCl solution (1M, 50 mL), dilute NaOH solution (1M, 50 mL) and brine (50 mL). The organic phase was dried (MgSO₄), filtered and concentrated in vacuo. Addition of methanol (˜2 mL) led to the precipitation of the title compound, which was obtained as an off-white solid after filtration. δ_{H} (d₆ DMSO): 3.08-3.21 (2H, m), 4.77 (1H, m), 6.91 (1H, d), 6.97 (1H, m), 7.20-7.25 (2H, m), 7.40 (2H, apps), 8.81 (1H, s), 9.90 (1H, d), 10.41 (1H, s), 12.22 (1H, br s); m/z (ES⁺)=340.92 [M+H]⁺; RT=3.42 min.

### Example 68

### 6-Chloro-1H-pyrrolo[3,2-c]pyridine-2-carboxylic acid (7-chloro-2-oxo-1,2,3,4-tetrahydroquinolin-3-yl)amide

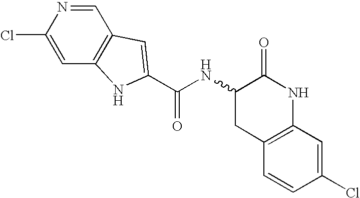

To a solution of 6-chloro-1H-pyrrolo[3,2-c]pyridine-2-carboxylic acid (Preparation 14, 40 mg, 0.20 mmol), EDCI (39.5 mg, 0.21 mmol) and HOBt monohydrate (31 mg, 0.20 mmol) in DMF (3 mL) was added 3-amino-7-chloro-3,4-dihydro-1H-quinolin-2-one (Preparation 19, 43.8 mg, 0.20 mmol) and DIPEA (78 μL, 0.45 mmol). The resulting solution was stirred for 12 h at rt before the reaction mixture was partitioned between EtOAc (50 mL) and water/brine (100 mL, 1:1). The layers were separated and the aqueous phase extracted with EtOAc (3×50 mL), then the combined organics were washed with dilute HCl solution (1M, 50 mL), dilute NaOH solution (1M, 50 mL) and brine (50 mL). The organic phase was dried (MgSO₄), filtered and concentrated in vacuo. Addition of methanol (˜2 mL) led to the precipitation of the title compound, which was obtained as an off-white solid after filtration. δ_{H} (d₆ DMSO): 3.12-3.18 (2H, m), 4.77 (1H, m), 6.93 (1H, d), 7.02 (1H, dd), 7.27 (1H, d), 7.40 (2H, m), 8.81 (1H, s), 9.01 (1H, d), 10.52 (1H, s), 12.21 (1H, br s); m/z (ES⁺)=374.85 [M+H]⁺; RT=3.40 min.

### Example 69

### 5-Chloro-1H-pyrrolo[2,3-c]pyridine-2-carboxylic acid (2-oxo-1,2,3,4-tetrahydro-[1,5]naphthyridin-3-yl)amide

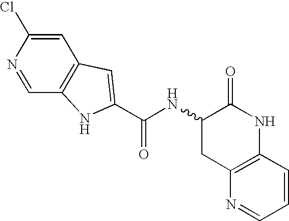

To a solution of 5-chloro-1H-pyrrolo[2,3-c]pyridine-2-carboxylic acid (Preparation 3, 50 mg, 0.25 mmol), EDCI (60 mg, 0.31 mmol) and HOBt monohydrate (40 mg, 0.26 mmol) in DMF (5 mL) was added of 3-amino-3,4-dihydro-1H-[1,5]naphthyridin-2-one dihydrochloride (WO 03/074532, 60 mg, 0.25 mmol) and DIPEA (186 μL, 1.07 mmol) and the resulting solution stirred for 12 h at rt. After concentration in vacuo the residue was partitioned between THF (50 mL) and carbonate buffer (100 mL, pH 10.5). The layers were separated and the aqueous phase extracted with THF (3×50 mL). The combined THF fractions were washed with brine (50 mL), dried (MgSO₄), filtered and concentrated. Addition of methanol (˜2 mL) led to the precipitation of the title compound, which was obtained as an off-white solid after filtration. δ_{H} (d₆ DMSO): 3.23 (1H, dd), 3.38 (1H, dd), 4.95 (1H, m), 7.25 (3H, m), 7.80 (1H, s), 8.14 (1H, m), 8.61 (1H, s), 9.11 (1H, d), 10.50 (1H, s), 12.37 (1H, br s); m/z (ES⁺)=342.03 [M−2HCl+H]⁺; RT=2.40 min.

### Examples 70-73

The following compounds were prepared according to the method of Example 69 from chloro-1H-pyrrolo[2,3-c]pyridine-2-carboxylic acid (Preparation 3) and the appropriate amine:

| | | | RT | |
|---|---|---|---|---|
| Ex | Structure | Name | (min) | M/z (ES⁺) |
| 70 | 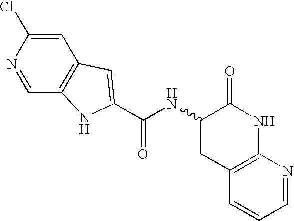 | 5-Chloro-1H-pyrrolo[2,3-c]pyridine-2-carboxylic acid(2-oxo-1,2,3,4-tetrahydro-[1,8]naphthyridin-3-yl)amide | 2.87 | 342.06[M + H]⁺ |
| 71 | 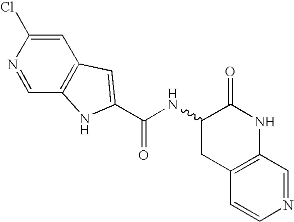 | 5-Chloro-1H-pyrrolo[2,3-c]pyridine-2-carboxylic acid(2-oxo-1,2,3,4-tetrahydro-[1,7]naphthyridin-3-yl)amide | 2.40 | 341.99[M + H]⁺ |
| 72 | 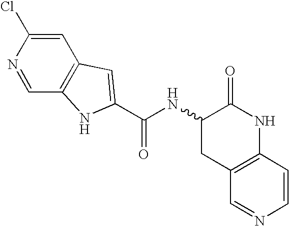 | 5-Chloro-1H-pyrrolo[2,3-c]pyridine-2-carboxylic acid(2-oxo-1,2,3,4-tetrahydro-[1,6]naphthyridin-3-yl)amide | 2.32 | 342.00[M + H]⁺ |
| 73 | 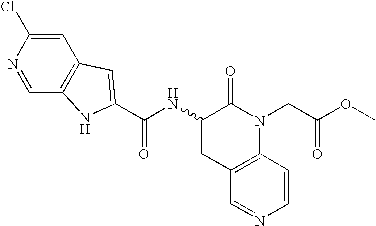 | {3-[(5-Chloro-1H-pyrrolo]2,3-c]pyridine-2-carbonyl)amino]-2-oxo-3,4-dihydro-2H-[1,6]naphthyridin-1-yl}-acetic acid methyl ester | 2.59 | 414.03[M + H]⁺ |

### Example 74

### 5-Chloro-1H-pyrrolo[2,3-c]pyridine-2-carboxylic acid [1-(2-hydroxyethyl)-2-oxo-1,2,3,4-tetrahydro-[1,5]naphthyridin-3-yl]amide

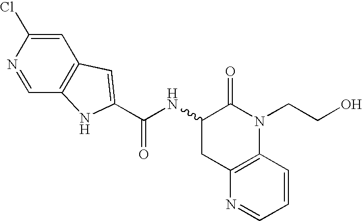

To a solution of 5-chloro-1H-pyrrolo[2,3-c]pyridine-2-carboxylic acid {1-[2-(tert-butyldimethylsilanyloxy)ethyl]-2-oxo-1,2,3,4-tetrahydro-[1,5]naphthyridin-3-yl}amide (Preparation 63, 176 mg, 0.35 mmol) in THF (10 mL) was added tetrabutylammonium fluoride (0.6 mL, 1N solution in THF). After 2 h the solvent was removed in vacuo. Purification of the residue by flash chromatography on silica gel (eluent: DCM/methanol: 9/1) gave the title compound as a colourless oil. δ_{H} (d₆ DMSO): 3.18 (1H, dd), 3.40 (1H, dd), 3.61 (2H, m), 3.93, 4.03 (2H, 2×m), 4.89 (1H, appt), 4.96 (1H, m), 7.28 (1H, s), 7.34 (1H, dd), 7.73 (1H, d), 7.80 (1H, s), 8.20 (1H, d), 8.60 (1H, s), 9.16 (1H, d), 12.38 (1H, br s); m/z (ES⁺)=386.04 [M+H]⁺; RT=2.67 min.

### Example 75

### {3-[(5-Chloro-1H-pyrrolo[2,3-c]pyridine-2-carbonyl)amino]-2-oxo-3,4-dihydro-2H-[1,5]naphthyridin-1-yl}acetic acid methyl ester

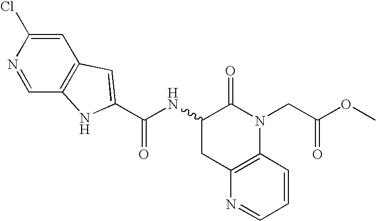

The title compound was prepared according to the method of Example 69 from chloro-1H-pyrrolo[2,3-c]pyridine-2-carboxylic acid (Preparation 3) and 3-amino-2-oxo-3,4-dihydro-2H-[1,5]naphthyridin-1-yl)acetic acid methyl ester (Preparation 60). δ_{H} (d₆ DMSO): 3.22 (1H, dd), 3.48 (1H, dd), 3.69 (3H, s), 4.70, 4.84 (2H, 2d), 5.00 (1H, ddd), 7.27 (1H, s), 7.36 (1H, dd), 7.51 (1H, d), 7.80 (1H, s), 8.24 (1H, d), 8.60 (1H, s), 9.23 (1H, d), 12.37 (1H, br s); m/z (ES⁺)=414.05 [M+H]⁺; RT=2.90 min.

### In Vitro GP Activity

### Materials

α-D-Glucose-1-phosphate (disodium salt), Glycogen, D-Glucose, Malachite Green Hydrochloride, Ammonium Molybdate tetrahydrate, BSA, HEPES and rabbit muscle phosphorylase a (P1261) were purchased from Sigma. All other reagents were analytical grade.

### Method

### Glycozen Phosphorylase Assay In Vitro:

An assay for glycogen phosphorylase activity in the reverse direction was developed based on the method described by Engers et al., Can. J. Biochem., 1970, 48, 746-754]. Rabbit muscle glycogen phosphorylase α (Sigma) was reconstituted at a stock concentration of 100 μg/mL in 25 mM Tris/HCl. The pH was measured in a 96-well plate in a final volume of 100 μL containing 50 mM Hepes pH 7.2, 7.5 mM glucose, 0.5 mM glucose-1-phosphate and 1 mg/mL glycogen. After incubation at 30° C. for 30 min, the inorganic phosphate released from glucose-1-phosphate was measured by the addition of 150 μL of malachite green/molybdate solution prepared as follows: 5 mL of 4.2% ammonium molybdate in 4N HCl, 15 mL of 0.045% malachite green, 50 μL of Tween 20. Following a 30 min incubation at rt, the absorbance was measured at 620 nm. For IC₅₀ determination, 10 μL of a serial dilution of compound (100 μM to 0.004 μM) in DMSO was added to each reaction in duplicate with the equivalent concentration of DMSO added to the control uninhibited reaction. Dose response curves were then obtained by plotting % inhibition versus log₁₀ compound concentration. IC₅₀ is defined as the concentration of compound achieving 50% inhibition under the assay conditions described.

The Examples have an IC₅₀ of <1 mM. It is advantageous that the measured IC₅₀ be lower than 100 μM. It is still more advantageous for the IC₅₀ to be lower than 50 μM. It is even more advantageous for the IC₅₀ to be lower than 5 μM. It is yet more advantageous for the IC₅₀ to be lower than 0.5 μM.

What is claimed is:

1. A compound of Formula (I): 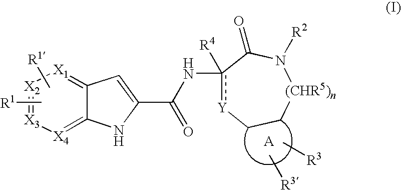
or a stereoisomer, or a pharmaceutically acceptable salt thereof, wherein:
one of X₁, X₂, X₃ and X₄ is N and the others are C; [symbol]
is a single or double bond;
when [symbol] is a single bond Y is CHR⁶, NH, O, S, SO₂, CHR⁶⁰, CHR⁶S, CHR⁶SO₂, CHR⁶CO or CH₂CHR⁶; and when [symbol] is a double bond Y is CR⁶ or N;
A is aryl or heteroaryl;
R¹ and R^{1′} are independently selected from hydrogen, halogen, hydroxy, cyano, C₁₋₆alkyl, C₁₋₆alkoxy, fluoromethyl, difluoromethyl, trifluoromethyl, C₂₋₆alkenyl, C₂₋₆alkynyl, aryl, —C₁₋₆alkylaryl, —C₁₋₆alkylheteroaryl and aryloxy;
R² is hydrogen, C₁₋₆alkyl optionally substituted by cyano, O—C₁₋₄alkyl-OR⁷, OR⁷, COOR⁷, CONR⁸R⁹, CONR⁸OR⁹, C(NH₂)═NOH, NR¹⁶R¹⁷, NHC(O)OR¹⁶, NHS(O)₂R¹⁸, NHC(O)R¹⁸, SR¹⁶, S(O)R¹⁸ or S(O)₂R¹⁸; or R² is C₁₋₄alkyl-CONR¹⁰R¹¹, C₂₋₆alkenyl, aryl, —C₁₋₆alkylaryl, —C₁₋₆alkylheterocyclyl or —C₁₋₆alkylheteroaryl;
R³ and R^{3′} are independently selected from hydrogen, halogen, hydroxy, cyano, C₁₋₄alkyl, C₁₋₄alkoxy, fluoromethyl, difluoromethyl, trifluoromethyl, ethenyl and ethynyl;
when [symbol] is a single bond R⁴ is hydrogen, C₁₋₆alkyl, aryl, or C₂₋₆alkenyl or when [symbol] is a double bond R⁴ is absent;
R⁵ and R⁶ are independently selected from hydrogen, C₁₋₆alkyl, aryl, C₂₋₆alkenyl, cyano, tetrazole, COOR¹², CONR¹²R¹³ and CONR¹²OR¹³;
R⁷, R⁸ and R⁹ are independently selected from hydrogen and C₁₋₄alkyl;
R¹⁰ and R¹¹ are independently selected from hydrogen, C₁₋₄alkyl optionally substituted by OR⁷, COOR⁷ or NR¹⁴R¹⁵, aryl, heteroaryl, C₃₋₇cycloalkyl, heterocyclyl, —C₁₋₄alkylaryl, —C₁₋₄alkylheteroaryl, —C₁₋₄alkylC₃₋₇cycloalkyl or —C₁₋₄alkylheterocyclyl wherein any of the rings is optionally substituted by 1 or 2 substituents independently selected from halogen, hydroxy, cyano, C₁₋₄alkyl, C₁₋₄alkoxy, fluoromethyl, difluoromethyl and trifluoromethyl;
or R¹⁰ and R¹¹ together with the nitrogen to which they are attached form a 4- to 7-membered heterocycle optionally containing a further heteroatom selected from N and O, which heterocycle is optionally substituted by C₁₋₄alkyl, halo, OR⁷ or COR⁷, or two bonds on a ring carbon of the heterocycle optionally can form an oxo (═O) substituent;
R¹² and R¹³ are independently selected from hydrogen, C₁₋₄alkyl, aryl, —C₁₋₄alkylaryl and —C₁₋₄alkylheteroaryl;
or R¹² and R¹³ may be cyclised to form an optionally substituted 4- to 7-membered heterocycle;
R¹⁴ and R¹⁵ are independently selected from hydrogen and C₁₋₄alkyl;
or R¹⁴ and R¹⁵ together with the nitrogen to which they are attached form a 4- to 7-membered heterocycle optionally containing a further heteroatom selected from N and O, which heterocycle is optionally substituted by C₁₋₄alkyl, halo, OR⁷ or COR⁷, or two bonds on a ring carbon of the heterocycle optionally can form an oxo (═O) substituent;
R¹⁶ and R¹⁷ are independently selected from hydrogen and C₁₋₄alkyl;
R¹⁸ is C₁₋₄alkyl; and
n is 0 or 1.

2. A compound according to claim 1 of Formula (Ia): 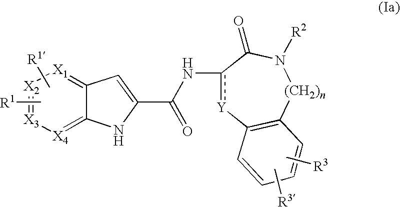
or a stereoisomer, or a pharmaceutically acceptable salt thereof, wherein:
one of X₁, X₂, X₃ and X₄ is N and the others are C; [symbol]
is a single or double bond;
when [symbol] is a single bond Y is CH₂, NH or O; and when [symbol] is a double bond Y is CH or N;
R¹ and R^{1′} are independently selected from hydrogen, halogen, hydroxy, cyano, C₁₋₄alkyl, C₁₋₄alkoxy, fluoromethyl, difluoromethyl, trifluoromethyl, ethenyl and ethynyl;
R² is hydrogen, C₁₋₄alkyl optionally substituted by cyano, O—C₁₋₄alkyl-OR⁴, OR⁴, COOR⁴, CONR⁵R⁶, CONR⁵OR⁶, C(NH₂)═N(OH), NR¹⁶R¹⁷, NHC(O)OR¹⁶, NHS(O)₂R¹⁸, NHC(O)R¹⁸, SR¹⁶, S(O)R¹⁸ or S(O)₂R¹⁸; or R² is C₁₋₄alkyl-CONR⁷R⁸ or —C₁₋₄alkylheterocyclyl;
R³ and R^{3′} are independently selected from hydrogen, halogen, hydroxy, cyano, C₁₋₄alkyl, C₁₋₄alkoxy, fluoromethyl, difluoromethyl, trifluoromethyl, ethenyl and ethynyl;
R⁴, R⁵ and R⁶ are independently selected from hydrogen and C₁₋₄alkyl;
R⁷ and R⁸ are independently selected from hydrogen, C₁₋₄alkyl optionally substituted by OR⁴, COOR⁴ or NR⁹R¹⁰, aryl, heteroaryl, C₃₋₇cycloalkyl, heterocyclyl, —C₁₋₄alkylaryl, —C₁₋₄alkylheteroaryl, —C₁₋₄alkylC₃₋₇cycloalkyl or —C₁₋₄alkylheterocyclyl wherein any of the rings is optionally substituted by 1 or 2 substituents independently selected from halogen, hydroxy, cyano, C₁₋₄alkyl, C₁₋₄alkoxy, fluoromethyl, difluoromethyl and trifluoromethyl;
or R⁷ and R⁸ together with the nitrogen to which they are attached form a 4- to 7-membered heterocycle optionally containing a further heteroatom selected from N and O, which heterocycle is optionally substituted by C₁₋₄alkyl, halo, OR⁴ or COR⁴, or two bonds on a ring carbon of the heterocycle optionally can form an oxo (═O) substituent;
R⁹ and R¹⁰ are independently selected from hydrogen and C₁₋₄alkyl;
or R⁹ and R¹⁰ together with the nitrogen to which they are attached form a 4- to 7-membered heterocycle optionally containing a further heteroatom selected from N and O, which heterocycle is optionally substituted by C₁₋₄alkyl, halo, OR⁴ or COR⁴, or two bonds on a ring carbon of the heterocycle optionally can form an oxo (═O) substituent;
R¹⁶ and R¹⁷ are independently selected from hydrogen and C₁₋₄alkyl;
R¹⁸ is C₁₋₄alkyl; and
n is 0 or 1.

3. A compound according to claim 1 or 2, or a stereoisomer, or a pharmaceutically acceptable salt thereof, wherein X₃ is N.

4. A compound according to any one of the preceding claims, or a stereoisomer, or a pharmaceutically acceptable salt thereof, wherein R¹ and R^{1′} are each independently, halogen, cyano or hydrogen.

5. A compound according to claim 4, or a stereoisomer, or a pharmaceutically acceptable salt thereof, wherein one of R¹ and R^{1′} is hydrogen and the other is a 5-halo or 5-cyano group.

6. A compound according to any one of the preceding claims, or a stereoisomer, or a pharmaceutically acceptable salt thereof, wherein when R² is C₁₋₄alkyl-CO—NR⁵R⁶ or R² is C₁₋₆alkyl-CO—NR⁸R⁹ as the case may be it is CH₂—CO—NR⁵R⁶ or CH₂CO—NR⁸R⁹ as the case may be.

7. A compound according to any one of the preceding claims, or a stereoisomer, or a pharmaceutically acceptable salt thereof, wherein R³ and R^{3′} are independently selected from hydrogen, halogen, C₁₋₄alkyl, C₁₋₄alkoxy, and trifluoromethyl.

8. A compound according to claim 7, or a stereoisomer, or a pharmaceutically acceptable salt thereof, wherein at least one of R³ and R^{3′} is hydrogen.

9. A compound according to any one of the preceding claims, or a stereoisomer, or a pharmaceutically acceptable salt thereof, wherein n is 0.

10. A compound selected from any one of Examples 1 to 75, as the free base or a pharmaceutically acceptable salt thereof.

11. A composition comprising a compound according to any one of claims 1 to 10, or a stereoisomer, or a pharmaceutically acceptable salt thereof, and a pharmaceutically acceptable carrier.

12. A method of prophylactic or therapeutic treatment of hyperglycemia or diabetes comprising a step of administering an effective amount of the compound according to any one of claims 1 to 10, or a stereoisomer, or a pharmaceutically acceptable salt thereof.

13. A method of prevention of diabetes in a human demonstrating pre-diabetic hyperglycemia or impaired glucose tolerance comprising a step of administering an effective prophylactic amount of the compound according to any one of claims 1 to 10, or a stereoisomer, or a pharmaceutically acceptable salt thereof.

14. A method of prophylactic or therapeutic treatment of hypercholesterolemia, hyperinsulinemia, hyperlipidemia, atherosclerosis or myocardial ischemia comprising a step of administering an effective amount of the compound according to any one of claims 1 to 10, or a stereoisomer, or a pharmaceutically acceptable salt thereof.

15. A method of cardioprotection comprising a step of administering to a subject in need thereof an effective amount of a compound of a compound according to any one of claims 1 to 10, or a stereoisomer or a pharmaceutically acceptable salt thereof.

16. A process for the production of a compound of Formula (I) according to claim 1, comprising coupling a carboxylic acid of Formula (II), or a protected or activated derivative thereof, with an amine of Formula (III): [chemical structure]
